(19) United States
(12) Patent Application Publication
(10) Pub. No.: US 2007/0275935 A1
(43) Pub. Date: Nov. 29, 2007

(54) Estratriene Derivatives

(76) Inventors: Alastair George Stewart, Victoria (AU); David James Mcallister, Victoria (AU); John Nicholas Lambert, Victoria (AU)

Correspondence Address: FINNEGAN, HENDERSON, FARABOW, GARRETT & DUNNER;LLP, 901 NEW YORK AVENUE, NW, WASHINGTON, DC, 20001-4413, US

(21) Appl. No.: 10/556,362

(22) PCT Filed: May 13, 2004
(86) PCT No.: PCT/AU04/00630

§ 371 Date: Mar. 19, 2007

Related U.S. Application Data
(60) Provisional application No. 60/470,379, filed on May 13, 2003.

(51) Int. Cl. A61K 31/58; A61K 31/56; C07J 43/00; C07J 41/00

(52) U.S. Cl. 514/169; 514/176; 514/182; 540/107; 552/516

(57) ABSTRACT

Compounds and methods for modulating mesenchymal cell function, for instance smooth muscle and fibroblast proliferation or cytokine expression, and for treating conditions associated with mesenchymal cell function, for instance airway hyperresponsiveness associated with asthma. The compounds also suppress inflammation. The compounds are a class of estratriene derivates, and includes various derivatives of 2-methoxyestradiol comprising a group A, including a substituted aromatic substituent in the 2-, 6- or 17-position.

### RELATED APPLICATIONS

This application claims priority from U.S. 60/470,397, the entire disclosure of which is incorporated by reference.

### FIELD OF THE INVENTION

The present application relates to compounds and methods for modulating mesenchymal cell function, for instance smooth muscle and fibroblast proliferation or cytokine expression, and for treating conditions associated with mesenchymal cell function, for instance airway hyperresponsiveness associated with asthma. The compounds of the present application are a class of estratriene derivatives.

### BACKGROUND OF THE INVENTION

Asthma is a disease characterised by a variable degree of airway obstruction, chronic inflammation, changes in airway wall structure that are referred to as airway wall remodelling (AWR), and airway hyper-responsiveness (AHR). The AHR is usually measured by artificial induction of airway obstruction by methacholine or histamine or other provocative stimuli such as cold air or bradykinin. AHR is characterised by a leftward shift in the concentration-response curve for airway stimulus-induced obstruction and a loss of the plateau in response to these non-specific stimuli.

Airway obstruction, such as that which occurs in asthma or chronic obstructive pulmonary disease may resolve itself spontaneously or upon treatment with agents. The three main classes of agents currently in use for treating asthma are the preventer drugs (anti-inflammatory agents), relievers and symptom controllers. All of these drugs aim to reduce airway obstruction and/or inflammation. The preventer drugs lessen the likelihood and/or the intensity of airway obstruction by anti-inflammatory actions. The reliever drugs produce a rapid reversal of smooth muscle shortening and therefore relieve bronchospastic airway obstruction. The symptom controllers produce a persistent bronchodilator response that lessens the likelihood of airway obstruction reaching a level that would require the use of reliever medication.

Each of the known treatments has significant adverse effects. Combinations of the above therapies successfully control the symptoms of asthma in many, but not all patients. Patients with severe asthma constitute less than 10% of the total population of asthmatics, but are thought to account for more than 70% of the health budget cost of asthma, and are the group at most risk of hospitalisation and death from asthma. Thus, there is a clear need for new therapies to control and prevent the symptoms of this disease.

The three classes of agents currently available do not specifically target the causes of airway wall remodelling (AWR); however, AWR has been identified as a possible new target for therapy of asthma. There is extensive evidence supporting the hypothesis that airway wall remodelling is a major factor in the development of AHR. An increase in the volume of airway occupied by smooth muscle, fibroblasts and extracellular matrix is thought to contribute to the thickening of the airway walls in AWR. At least one cause of this increase in volume is hyperproliferation by airway smooth muscle cells. The consequence of such thickening is increased airway narrowing for a given amount of smooth muscle contraction. In addition, AWR reduces the load on smooth muscle cells in the airways, allowing a greater amount of smooth muscle cell shortening for a given degree of activation of the contractile apparatus. The structural changes in AWR are therefore able to account for the non-specific nature of AHR. It follows that anti-asthma agents that are able to prevent or reverse the remodelling have the potential to reduce AHR, and consequently to reduce the level of airway obstruction that occurs as a result of smooth muscle contraction.

It is an object of the present application to provide agents and methods that are capable of modulating the function of smooth muscle cells or fibroblasts, and thereby providing potential agents for treating conditions associated with smooth muscle cell or fibroblast function, such as asthma.

### SUMMARY OF THE INVENTION

The present application is directed to novel estratriene derivatives and methods for their synthesis and use. The novel estratriene derivatives may be used to modulate smooth muscle cell and/or fibroblast function, such as cell proliferation, extracellular matrix deposition, cytokine expression and contractility may be used in conditions involving smooth muscle cells and/or fibroblasts. These compounds have demonstrated a surprising selectivity in modulating the proliferation of and cytokine expression of smooth muscle and/or fibroblast cells.

One embodiment provides a compound of the Formula I: 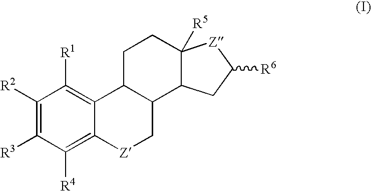
in which:
R¹ and R⁴ are each selected from the group consisting of H, R^{a}, —R^{c}R^{d}, —CN, —NO₂, -halo, OH, —OR^{a}, —OC(O)R^{a};
R² is selected from the group consisting of —OR^{b}, —(R^{c})ₙ-AR^{b}, —H, —R^{e}, —R^{c}R^{e}, —CH═NOH, —CH═NOR^{b}, —CH═NNR^{b}₂, —OH, —SR^{b}, —R^{b}, —CN, —R^{c}R^{d} and -halo, in which n is 0 or 1;
R³ is selected from the group consisting of —OH, —OR^{a}, —R^{c}OR^{b}, —H, -ester-R^{b};
R⁵ is methyl;
R⁶ is —H, —OH, —OR^{b} or -halo;
Z′ is A or >CH₂, >C═O, >C═N—OH, >C═N—OR^{b}, >C(R^{b})—OH, >C(R^{b})—CN; >C(R^{b})—NR^{b}₂, >CR^{b}₂, >C═N—NH₂, >C═N—NR^{b}₂, —O—, >N—R^{b}, >C(R^{b})—R^{c}—OR^{b}, >CR^{b}R^{e}, >CR^{b}—NR^{b}R^{e}, >C═N-ester-R^{a};
Z″ is A or >C═O, >C(H)OH, >C═N—OH, >C═N—OR^{b}, >C(R^{b})—OR^{b}, >C(R^{b})—R^{c}—OR^{b}, >C(H)—NR^{b}₂, >C(H)-halo, >CR^{b}₂, >C═N-ester-R^{a};
A is >C═N—O—X, >C═N—O—R^{c}—X, >C═N—NH—R^{c}—X, >C═N—NH—X, >C═N-ester-X;
X is an aromatic group substituted by one or more substituents Y, wherein Y is selected from —H, —NO₂, —CN, —SO₃H, —SO₃R^{a}, —CO—R^{b}, —⁺NR^{b}₃, —CO₂R^{b}, -halo, —CF₃, —CCl₃, tetrazole, imidazole, -aryl, -substituted aryl, —R^{a}, —NH₂, —NR^{a}₂, —OH, —OR^{a}, —R^{c}—CN, —R^{c}-halo, —NR^{b}COR^{b}, —R^{c}—NR^{b}₂, —R^{c}R^{d};
R^{a} is a straight chained, branched or cyclic alkyl, alkenyl, alkynyl, aralkyl, aralkenyl or aralkynyl;
R^{b} is H or straight chained, branched or cyclic alkyl, alkenyl, alkynyl, aralkyl, aralkenyl or aralkynyl;
R^{c} is a straight chained or branched C1-C10 alkylene, alkenylene or alkynylene;
R^{d} represents one or more substituents selected from —OH, —NH₂, -halo, —CF₃, —CN, —COOR^{a}, —SR^{b};
R^{e} is acyl;
N is 0 or 1;
with the proviso the compound contains at least one group A, and that when R¹ and R⁴ are H, R² is OR^{a}, R³ is OH, R⁶ is —H and Z″ is OH or NH₂, then A at Z′ is not >C═N—NH—SO₂—X;
or a salt, hydrate, pro-drug, isomer, tautomer and/or derivative thereof. According to one embodiment, at least one of Z′ and Z″ is A. In other words, according to this embodiment, A is at one or both of positions 6 and 17.

According to another embodiment, although the class of “substituents” Y includes H, when the aromatic group is phenyl, the class of possible substituents Y suitably excludes H.

In one embodiment the compound disclosed has activity in modulating the function of fibroblasts and/or smooth muscle cells. The compound may modulate cell function by suppressing proliferation. In other embodiments the compound may modulate cell function by influencing one or more of extracellular matrix deposition by cells, cell migration, cell cytokine expression or cell contractility.

In one embodiment, the compound is able to suppress airway hyperresponsiveness, such as airway hyperresponsiveness exhibited in asthma.

In another embodiment, the compound suppresses fibrosis, such as pulmonary fibrosis, or pulmonary inflammation.

According to an embodiment, the compound has specific activity in modulating mesenchymal cell function.

The present disclosure also provides a method of synthesising a compound of Formula I, as defined above, comprising the step of reacting a ketone or aldehyde precursor of Formula II, III or IV: 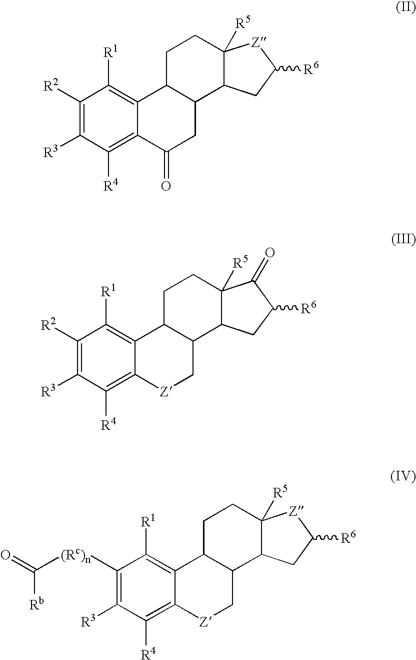
in which R¹, R², R³, R⁴, R⁵, R⁶, R^{b}, R^{c}, n, Z′ and Z″ are as defined in Formula I,
with an amine of the formula H₂N—O—X, H₂N—O—R^{c}—X, H₂N—NH—R^{c}—X, H₂N—NH—X or H₂N-ester-X, in which X is as defined in Formula I,
to form the compound of Formula I.

According to the present application there is also provided a pharmaceutical composition comprising the compound of Formula I defined above and a pharmaceutically acceptable carrier.

According to the present application there is also provided the use of a compound of Formula I defined above as an agent for modulating smooth muscle cell and/or fibroblast function. In one embodiment, the use is as an antiproliferative or anti-inflammatory agent. The present application also provides for the use of a compound of Formula I as an airway hyperresponsiveness suppressor or as an anti-asthmatic agent.

In another embodiment, the compound is used as an agent for suppressing fibrosis, such as pulmonary fibrosis. In a further embodiment the compound of Formula I is used as an agent for suppressing pulmonary inflammation.

In one embodiment the compound is used as an agent having specific activity in modulating mesenchymal cell function. According to the present application there is provided a method for the treatment of a condition associated with smooth muscle cell and/or fibroblast function, which comprises administering a therapeutically effective amount of a compound of Formula I to a subject in need thereof. In certain specific embodiments, the condition is associated with airway smooth muscle cell or pulmonary fibroblast function. The cell function may be selected from cell proliferation, cell cytokine expression, extracellular matrix deposition by cells, cell migration or cell contractility.

According to the present application there is also provided the use of a compound of Formula I in the treatment of airway hyperresponsiveness. In a preferred embodiment, the compound is used in the treatment of asthma.

According to the present application there is also provided the use of a compound of Formula I in the suppression of fibrosis such as pulmonary fibrosis.

According to the present application there is also provided the use of a compound of Formula I in the suppression of pulmonary inflammation.

According to the present application there is provided the use of a compound of Formula I in the manufacture of a medicament for the treatment of a condition associated with smooth muscle cell and/or fibroblast function.

It will be appreciated by those skilled in the art that the activities of the agents established in this application include but are not limited to the suppression of smooth muscle proliferation, migration and cytokine synthesis as well as the suppression of fibroblast proliferation and the suppression of inflammation. In view of these activities, it is expected that agents will be active in the treatment of allergic and inflammatory diseases such as rheumatoid arthritis, allergic dermatitis, allergic rhinitis, asthma, chronic obstructive pulmonary disease, adult respiratory distress syndrome, chronic infection and sepsis. The reduction of fibroblast proliferation also suggests that agents are expected to have activity in the treatment of fibrotic conditions including pulmonary fibrosis of diverse types, scarring and visceral adhesions/fibroids.

Preferred compounds of Formula I are those in which one or more of the following apply:
i. Y is preferably a deactivating group selected from the group —NO₂, —CN, —SO₃H, —SO₃R^{a}, —CO—R^{b}, —⁺NR^{b}₃, —CO₂R^{b}, -halo, —CF₃—CCl₃, tetrazole and imidazole, more preferably —NO₂, —CN, —CO₂R^{b}, -halo, —CF₃—CCl₃, tetrazole or imidazole;
ii. X is preferably phenyl, naphthyl or pyridyl and most preferably phenyl;
iii. X preferably includes 1 or 2 substituents Y, more preferably one, and most preferably when X is phenyl, the single substituent Y is in the 4-position;
iv. The group R^{c} in A, when present, is preferably alkylene, more preferably C1-C6 alkylene, and most preferably —CH₂— or —CH₂CH₂—;
v. A is preferably >C═N—O—X, >C═N—O—R^{c}—X or >C═N—NH—R^{c}—X, more preferably >C═N—O—X or >C═N—O—R^{c}—X;
vi. When Z″ is A, Z′ is preferably >CH₂, >C═O, >C═N—OH or >C═N—OR^{b};
vii. When Z′ is A, Z″ is preferably >C═O, >C(H)OH, >C═N—OH or >C═N—OR^{b}, more preferably >C═O, >C(H)OH or >C═N—OH;
viii. R² is preferably —OR^{b}, -AR^{b}, —R^{e}, —R^{c}R^{e}, —CH═NOH, —CH═NOR^{b}, —CH═NNR^{b}₂, —OH, —SR^{b}, —R^{b} or —CN, more preferably —OR^{b}, most preferably —OMe;
ix. R³ is preferably —OH, —OR¹ or —R^{c}OR^{b}, most preferably —OH;
x. R¹ and R⁴ are each preferably H; and
xi. R⁶ is preferably H.

Each of the above groups of preferred features can be combined with each other. Certain combinations are particularly preferred, such as group iv. with groups i. and/or iii.

The present application further encompasses the following compounds:

Compounds of Formula (V): 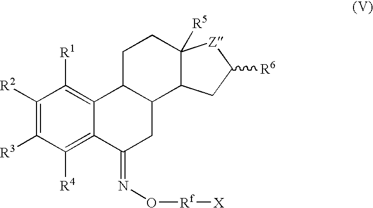
in which
R¹, R², R³, R⁴, R⁵, R⁶, Z″ and X are as defined for formula I, and
R^{f} is a direct bond or an alkylene group;

Compounds of Formula (VI): 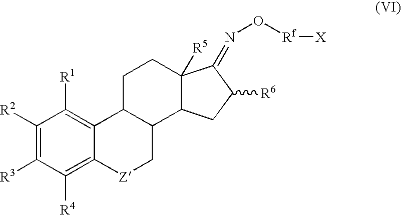
in which
R¹, R², R³, R⁴, R⁵, R⁶, Z′ and X are as defined for formula I, and
R^{e} is a direct bond or an alkylene group;

Compounds of Formula (VII): 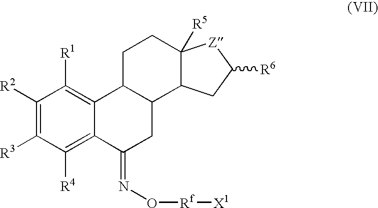
in which
R¹, R², R³, R⁴, R⁵, R⁶ and Z″ are as defined in Formula I;
R^{f} is a direct bond or an alkylene group, and
X¹ is selected from aryl groups substituted by one or more substituents Y¹, wherein Y¹ is selected from —NO₂, —CN, —SO₃H, —SO₃R^{a}, —CO—R^{b}, —⁺NR^{b}₃, —CO₂R^{b}, -halo, —CF₃, —CCl₃, tetrazole, imidazole, -aryl, -substituted aryl, —R^{a}, —NH₂, —NR^{a}₂, —OH, —OR^{a}, —R^{c}—CN, —R^{c}-halo, —NR^{b}COR^{b}, —R^{c}—NR^{b}₂, R^{c}R^{d} and heteroaromatic groups substituted by one or more substituents Y, wherein Y is selected from —H, —NO₂, —CN, —SO₃H, —SO₃R^{a}, —CO—R^{b}, —⁺NR^{b}₃, —CO₂R^{b}, -halo, —CF₃, —CCl₃, tetrazole, imidazole, -aryl, -substituted aryl, —R^{a}, —NH₂, —NR^{a}₂, —OH, —OR^{a}, —R^{c}—CN, —R^{c}-halo, —NR^{b}COR^{b}, —R^{c}—NR^{b}₂, —R^{c}R^{d};

Compounds of Formula (VIII): 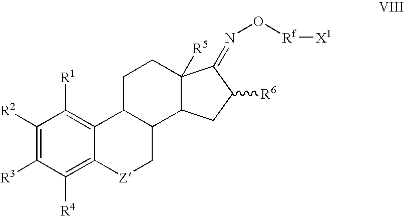
in which
R¹, R², R³, R⁴, R⁵, R⁶ and Z′ are as defined in Formula I;
R^{f} is a direct bond or an alkylene group, and
X¹ is selected from aryl groups substituted by one or more substituents Y¹, wherein Y¹ is selected from —NO₂, —CN, —SO₃H, —SO₃R^{a}, —CO—R^{b}, —⁺NR^{b}₃, —CO₂R^{b}, -halo, —CF₃, —CCl₃, tetrazole, imidazole, -aryl, -substituted aryl, —R^{a}, —NH₂, —NR^{a}₂, —OH, —OR^{a}, —R^{c}—CN, —R^{c}-halo, —NR^{b}COR^{b}, —R^{c}NR^{b}₂, —R^{c}R^{d}, and heteroaromatic groups substituted by one or more substituents Y, wherein Y is selected from —H, —NO₂, —CN, —SO₃H, —SO₃R^{a}, —CO—R^{b}, —⁺NR^{b}₃, —CO₂R^{b}, -halo, —CF₃, —CCl₃, tetrazole, imidazole, -aryl, -substituted aryl, —R^{a}, —NH₂, —NR^{a}₂, —OH, —OR^{a}, —R^{c}—CN, —R^{c}-halo, —NR^{b}COR^{b}, —R^{c}NR^{b}₂, —R^{c}R^{d};

Compounds of Formula (IX): 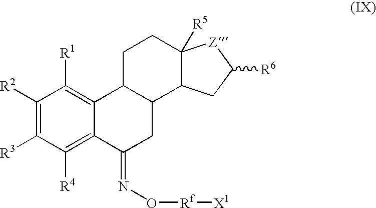
in which
R¹, R², R³, R⁴, R⁵ and R⁶ are as defined in Formula I,
R^{f} is a direct bond or an alkylene group,
X¹ is selected from aryl groups substituted by one or more substituents Y¹, wherein Y¹ is selected from —NO₂, —CN, —SO₃H, —SO₃R^{a}, —CO—R^{b}, —⁺NR^{b}₃, —CO₂R^{b}, -halo, —CF₃, —CCl₃, tetrazole, imidazole, -aryl, -substituted aryl, —R^{a}, —NH₂, —NR^{a}₂, —OH, —OR^{a}, —R^{c}—CN, —R^{c}-halo, —NR^{b}COR^{b}, —R^{c}—NR^{b}₂, —R^{c}R^{d}; and heteroaromatic groups substituted by one or more substituents Y, wherein Y is selected from —H, —NO₂, —CN, —SO₃H, —SO₃R^{a}, —CO—R^{b}, —NR^{b}₃, —CO₂R^{b}, -halo, —CF₃, —CCl₃, tetrazole, imidazole, -aryl, -substituted aryl, —R^{a}, —NH₂, NR^{a}₂, —OH, —OR^{a}, —R^{c}—CN, —R^{c}-halo, —NR^{b}COR^{b}, —R^{c}—NR^{b}₂, —R^{c}R^{d}; and
Z′″ is >C═O, >C(H)OH or >C═N—OH;

Compounds of Formula (X): 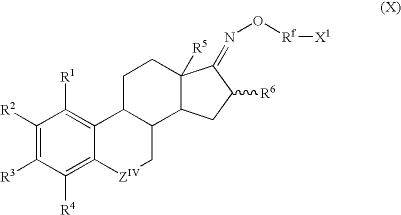
in which
R¹, R², R³, R⁴, R⁵ and R⁶ are as defined in Formula I,
R^{f} is a direct bond or an alkylene group,
X¹ is selected from aryl groups substituted by one or more substituents Y¹, wherein Y¹ is selected from —NO₂, —CN, —SO₃H, —SO₃R^{a}, —CO—R^{b}, —⁺NR^{b}₃, —CO₂R^{b}, -halo, —CF₃, —CCl₃, tetrazole, imidazole, -aryl, -substituted aryl, —R^{a}, —NH₂, —OH, —OR^{a}, —R^{c}—CN, —R^{e}-halo, —NR^{b}COR^{b}, —R^{c}—NR^{b}₂, —R^{c}R^{d}; and heteroaromatic groups substituted by one or more substituents Y, wherein Y is selected from —H, —NO₂, —CN, —SO₃H, —SO₃R^{a}, —CO—R^{b}, —⁺NR^{e}, —CO₂R^{b}, -halo, —CF₃, —CCl₃, tetrazole, imidazole, -aryl, -substituted aryl, —R^{a}, —NH₂, —NR^{a}₂, —OH, —OR^{a}, —R^{c}—CN, —R^{c}-halo, —NR^{b}COR^{b}, —R^{c}—NR^{b}₂, —R^{c}R^{d}; and
Z^{IV} is >CH₂, >C═O or >C═N—OH;

Compounds of Formula (XI): 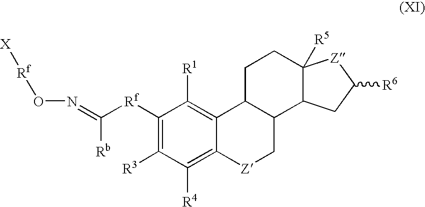
in which:
R¹, R², R³, R⁴, R⁵, R⁶, Z′, Z″, R^{b}, and X are as defined in Formula I, and
R^{f} is a direct bond or an alkylene group.

The present applicant has found that estratriene derivatives containing the substituent A defined above have activity in modulating smooth muscle and/or fibroblast cell function, and in particular cell proliferation, cell extracellular matrix deposition, cell migration and cytokine expression. Accordingly, the present application broadly encompass all estradiol derivatives containing the substituent A defined above, with the proviso that the compound is not 2-ethoxy-6-tosylhydrazone-estra-1,3,5(10)-triene-3,17β-diol.

### BRIEF DESCRIPTION OF THE FIGURES

FIG. 1 compares the effectiveness and potency of 2-methoxyestradiol (2MEO), with two compounds, 4NO (CP-DM-2-11-7) and 4NOM (CP-DM-3-91), in inhibiting thrombin-mediated cell division in cultured human airway smooth muscle cells.

FIG. 2 compares the effectiveness and potency of 2MEO, 4NO (CP-DM-2-11-7) and 4NOM (CP-DM-3-91) in inhibiting the proliferation of a type II airway epithelial cell line, A549, in response to 5% fetal calf serum.

FIG. 3 compares the effectiveness and potency of 2MEO and 4NO (CP-DM-2-11-7) in inhibiting the proliferation of an estrogen receptor-expressing breast tumour cell line, MCF7, in response to either 5% fetal calf serum or 300 pM epidermal growth factor.

FIG. 4 compares the effectiveness and potency of 2MEO and 4NO (CP-DM-2-11-7) in inhibiting the proliferation of cultured bovine aortic endothelial cells in response to 5% fetal calf serum.

FIG. 5 compares the effectiveness and potency of 4NO (CP-DM-2-11-7) and dexamethasone in inhibiting basic fibroblast growth factor-mediated proliferation of human airway smooth muscle cells cultured on tissue culture plastic.

FIG. 6 compares the effectiveness and potency of 4NO (CP-DM-2-11-7) and dexamethasone in inhibiting basic fibroblast growth factor-mediated proliferation of human airway smooth muscle cells cultured on collagen-coated silastic.

FIG. 7 compares the effectiveness and potency of 2MEO and 4NO (CP-DM-2-11-7)in inhibiting the interleukin-1 mediated release of granulocyte-macrophage colony-stimulating factor from cultured human airway smooth muscle cells.

FIG. 8 compares the effectiveness and potency of intraperitoneally administered 2MEO and 4NO (CP-DM-2-11-7) in inhibiting airway hyperresponsiveness to an intravenous challenge of the bronchoconstrictor methacholine in mice sensitised to ovalbumin.

FIG. 9 compares the effectiveness and potency of orally administered 2MEO and 4NO (CP-DM-2-11-7) in inhibiting airway hyperresponsiveness to an intravenous challenge of the bronchoconstrictor methacholine in mice sensitised to ovalbumin.

FIG. 10 compares the effectiveness and potency of 4NO (CP-DM-2-11-7) and 4NOM (“4NO-menox”) (CP-DM-3-91) in inhibiting the proliferation of pulmonary fibroblasts in response to basic fibroblast growth factor stimulation.

FIG. 11 demonstrates the effect of pre-incubation with 4NOM (CP-DM-3-91) on thrombin-mediated cyclin D1 protein expression by human airway smooth muscle cells.

FIG. 12 compares the effectiveness of 2MEO or 4NO (CP-DM-2-11-7) in inhibiting platelet-derived growth factor(BB)-induced migration of human airway smooth muscle cells.

### DETAILED DESCRIPTION OF THE INVENTION

For the purposes of this specification it will be clearly understood that the word “comprising” means “including but not limited to”, and that the word “comprises” has a corresponding meaning.

The compounds disclosed are a class of derivatives of 2-methoxyestradiol (2MEO). 2MEO and other derivatives of this compound and their activity in treating cancer have been explored previously.

The term “alkyl” used either alone or in compound words such as “aralkyl” refers to straight chain, branched chain or cyclic hydrocarbon groups having from 1 to 10 carbon atoms, preferably 1 to 6 carbon atoms, more preferably 1 to 4 carbon atoms. Illustrative of such alkyl groups are methyl, ethyl, propyl, isopropyl, butyl, isobutyl, sec-butyl, tert-butyl, pentyl, neopentyl, hexyl, cyclopropyl, cyclobutyl, cyclopentyl or cyclohexyl.

The term “alkenyl” used either alone or in compound words denotes linear, branched or mono- or poly-cyclic radicals having at least one carbon-carbon double bond of 2 to 20 carbon atoms, preferably 2 to 14 carbon atoms, more preferably 2 to 6 carbon atoms. Examples of alkenyl radicals include allyl, ethenyl, propenyl, butenyl, iso-butenyl, 3-methyl-2-butenyl, 1-pentenyl, cyclopentenyl, 1-hexenyl, 3-hexenyl, cyclohexenyl, 1-nonenyl, 1,3-butadienyl, and the like. The term “alkynyl” has a corresponding meaning.

The term “acyl” used either alone or in compound words denotes carbamoyl, aliphatic acyl group, acyl group containing an aromatic ring which is referred to as aromatic acyl or an acyl group containing a heterocyclic ring which is referred to as heterocyclic acyl having 1 to 20 carbon atoms, preferably 1 to 14 carbon atoms. Examples of acyl include carbamoyl; straight chain or branched alkanoyl, such as, formyl, acetyl, propanoyl, butanoyl, 2-methylpropanoyl, octanoyl; alkoxycarbonyl, such as, methoxycarbonyl, ethoxycarbonyl, t-butoxycarbonyl; cycloalkylcarbonyl, such as cyclohexylcarbonyl; alkylsulfonyl, such as, methylsulfonyl or ethylsulfonyl; alkoxysulfonyl, such as, methoxysulfonyl or ethoxysulfonyl; aroyl, such as, benzoyl, toluoyl or naphthoyl; aralkanoyl, such as phenylalkanoyl, for example, phenylacetyl or phenylpropanoyl, or naphthylalkanoyl, for example, naphthylbutanoyl; aralkenoyl, such as, phenylalkenoyl, for example, phenylpropenoyl or phenylmethacrylyl or naphthylalkenoyl, for example, naphthylpropenoyl; aralkoxycarbonyl, such as, phenylakoxycarbonyl, for example, benzyloxycarbonyl; aryloxycarbonyl, such as phenoxycarbonyl; aryloxyalkanoyl, such as phenoxypropionyl, arylcarbamoyl, such as phenylcarbamoyl; arylthiocarbamoyl, such as phenylthiocarbamoyl, arylglyoxyloyl, such as phenylglyoxyloyl; arylsulfonyl, such as phenylsulfonyl; heterocycliccarbonyl; heterocyclicalkanoyl, such as thienylacetyl, thiadiazolylacetyl or tetrazolylacetyl; heterocyclicalkenoyl, such as heterocyclicpropenoyl; or heterocyclicglyoxyloyl, such as thiazolylglyoxyloyl.

The term “heterocyclyl group” used either alone or in compound words refers to monocyclic or polycyclic heterocyclic groups containing at least one heteroatom atom selected from nitrogen, sulphur and oxygen. A small number of examples to illustrate the vast range of heterocycles within this term are as follows: pyrrolyl, pyrrolinyl, imidazolyl, pyrazolyl, pyridyl, pyrimidinyl, pyrazinyl, pyridazinyl, triazolyl, tetrazolyl, pyrrolidinyl, indolyl, benzimidazolyl, pyranyl, furyl, thienyl, oxazolyl, isoxazolyl oxadiazolyl, morpholinyl, benzoxazolyl, benzoxadiazolyl, thiazolyl, thiadiazolyl, thiazolidinyl, benzothiazolyl and benzothiadiazolyl.

Preferably the heterocyclyl is as an aromatic 5- or 6-membered heteromonocyclic group.

The term “aryl” used either alone or in compound words such as “aralkyl” or “aryl acyl” denotes a carbocyclic aromatic system containing one, two or three rings wherein such rings may be attached together in a pendent manner or may be fused. The term “aryl” embraces aromatic radicals such as phenyl, naphthyl, tetrahydronaphthyl, indane and biphenyl. Preferably, the aryl is phenyl.

The term “aromatic group” refers to aryl groups and aromatic heterocyclyl groups (ie heteroaromatic) groups and includes the specific heterocycles exemplified above.

The term “halo” refers to fluorine, chlorine, bromine or iodine.

The term “alkoxy” refers to straight chain or branched oxy-containing radicals preferably each having alkyl portions of 1 to about 6 carbon atoms. Examples of alkoxy include methoxy, ethoxy, propoxy, butoxy and tert-butoxy.

The term “optionally substituted” refers to a group which may or may not be further substituted with one or more groups selected from alkyl, alkenyl, alkynyl, aryl, aldehyde, halo, haloalkyl, haloalkenyl, haloalkynyl, haloaryl, hydroxy, alkoxy, alkenyloxy, aryloxy, benzyloxy, haloalkoxy, haloalkenyloxy, haloaryloxy, nitro, nitroalkyl, nitroalkenyl, nitroalkynyl, nitroaryl, nitroheterocyclyl, amino, alkylamino, dialkylamino, alkenylamino, alkynylamino, arylamino, diarylamino, benzylamino, dibenzylamino, acyl, alkenylacyl, alkynylacyl, arylacyl, acylamino, diacylamino, acyloxy, alkylsulphonyloxy, arylsulphenyloxy, heterocyclyl, heterocycloxy, heterocyclamino, haloheterocyclyl, alkylsulphenyl, arylsulphenyl, carboalkoxy, carboaryloxy, mercapto, alkylthio, benzylthio, acylthio, phosphorus-containing groups and the like.

The term “ester” is used herein in its broadest sense to refer to divalent groups containing at least one carbon, sulfur or phosphorus to oxygen double bond, and a single bond from the carbon, sulfur or phosphorus atom to another oxygen or a sulfur atom. Examples of organic ester groups include sulfonylates, sulfonates, phosphonates, phosphonothioates, carboxylates and thiolates (RCOS). Additionally, other functional groups may be contained within the ester group. These functional groups include thio, sulfanyl, sulfinyl, sulfonyl, sulfonatyl, phosphinyl, phosphonyl, phosphoryl, amino or mixtures thereof. Examples of esters containing these functional groups include the esters derived from phosphonothioic, thiophosphoric, sulfinylphosphoric, sulfatylamino, sulfonatylphosphinic and phosphoramidic acids.

Conditions associated with airway smooth muscle cell proliferation include asthma and related conditions such as airway wall remodelling and airway hyperresponsiveness, and also conditions unrelated to asthma but which are characterised by smooth muscle cell proliferation, such as chronic obstructive pulmonary disease or aberrant growth of smooth muscle cells leading to a neoplasm, tumour or a cancer.

The inhibition of airway smooth muscle cell proliferation encompasses the slowing or halting the cycle of smooth muscle cell division. In one embodiment the inhibition of proliferation is preferably not accompanied by increased smooth muscle cell death or loss of other normal smooth muscle cell functions such as contractility.

The term “selectivity” is used herein to describe the ability of a compound to influence the behavior of one or more cell types from a tissue to a greater extent than other cell types associated with that tissue.

For instance, selectivity of the inhibition of airway smooth muscle cell or fibroblast proliferation means that the rate of cell division of the cells is decreased by a greater proportion than in other cells associated with the airways and other tissues, such as endothelial cells, alveolar cells, epithelial cells, mast cells and breast tumour epithelial cells.

Methods of Synthesis

The compounds disclosed containing the group A in a selected position or positions can be prepared by reacting the appropriate estratriene-based precursor having a carboxy group in place of A with the appropriate amine in a condensation reaction. In the following we have set out standard procedures for the synthesis of the substituted aryl amines (H₂N—O—X, H₂N—O—R^{c}—X, H₂N—NH—R^{c}—X, H₂N—NH—X or H₂N-ester-X), and thereafter we will describe the various methods for diversifying the estratriene precursor.

Substituted arylamines (H₂N—O—X, H₂N—O—R^{c}—X, H₂N—NH—R^{c}—X, H₂N—NH—X or H₂N-ester-X)

Substituted aryl hydroxylamines (eg. H₂N—O—X and H₂N—O—R^{c}—X) can readily be synthesised by several methods. The preferred method of synthesis is via the key phthalimido intermediate of aryl substituted hydroxylamines. In this instance N-hydroxyphthalimide acts via nucleophilic attack on substituted aryl and arylalkyl halides.[1] Any number of other leaving groups can be used in place of the halide. Subsequent removal of the phthalimide group by treatment with hydrazine gives the substituted aryl hydroxylamine, which is often isolated as its HCl salt.

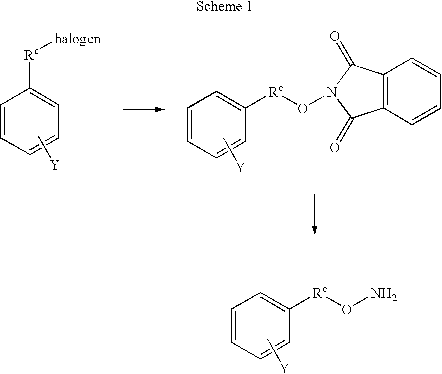

Another route to these intermediates is through the copper mediated coupling of substituted phenylboronic acids with N-hydroxyphthalimide.[2] Again subsequent removal of the phthalimide group with hydrazine gives the expected aryloxyamine.

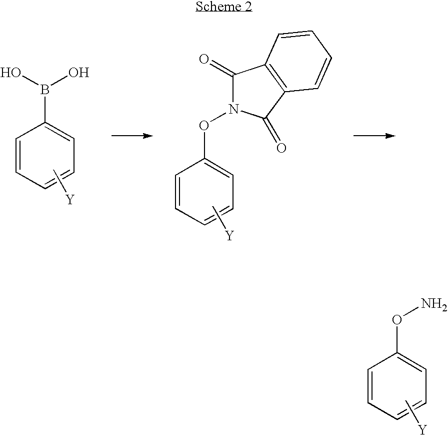

Yet another method of synthesis of substituted aryl hydroxylamines is the nucleophilic attack on a substituted fluorobenzene with deprotonated ethyl acetoxyhydroxamate followed by hydrolysis with perchloric acid.[3]

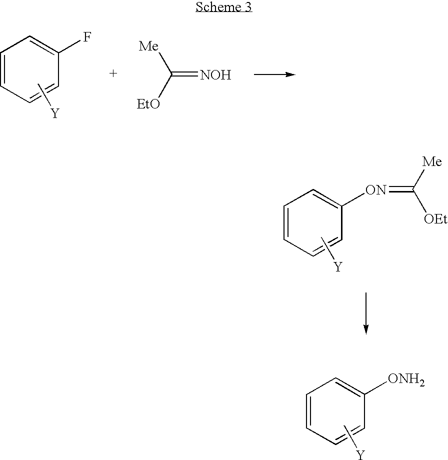

These examples are for the case of phenyl rings, but several methods for the production of naphthyl and pyridyl substituted hydroxylamines also exist.[4-6]

Once these substituted hydroxylamines have been synthesised, they can be readily used in condensation reactions with both ketones and aldehydes contained within the base estra-1,3,5(10)-triene steroid structure to form substituted aryl oximes.

A large range of substituted aryl hydrazines are commercially available (eg 2,4-dinitrophenylhydrazine). A large range of substituted aryl sulfonyl hydrazines (H₂N—NH—SO₂—X) and substituted aryl sulfonyl hydroxylamines are also commercially available. Alternatively, a simple method of generating substituted aryl hydrazines involves the use of well established reductive amination chemistry as exemplified by Malachowski et al.[28]. A substituted aryl aldehyde can be subjected to nucleophilic attack from hydrazine and the product reduced to give the desired substituted aryl hydrazine.

Reaction of these hydrazines with the appropriate precursor compound II, III or IV will then yield the corresponding hydrazide, sulfonic acid hydrazide or sulfonyl oxime. The latter two of these three groups are examples of the ester-containing compounds of the present application.

Diversification of estra-1,3,5(10)-triene to Form the Precursor Compounds II, III and IV

We now turn to the various methods that may be used to obtain the precursor estratriene containing a carboxy group in place of A, and any of the range of substituents shown to be possible in position R¹, R², R³, R⁴, R⁶, Z′ and Z″.

(A) Diversification at R¹, R², R³ and R⁴

Many commercially available estra-1,3,5(10)-triene compounds can be readily obtained with a great variance of substitution on the aromatic A-ring of the molecule. Many of these commercial compounds also have the ketone functionality present at Z″ which can be condensed with the substituted arylamine to form the compound of formula I. These compounds could alternatively be subjected to further diversification using the techniques shown below before condensation with the substituted aryl amine.

As examples, the following compounds are commercially available from Steraloids Inc.:

17-OH Compounds

1-methylestradiol
2,4-dibromoestradiol
2,4-dinitroestradiol
2-bromoestradiol
2-ethoxyestradiol
2-fluoroestradiol
2-hydroxyestradiol
2-hydroxyestradiol-3-methyl ether
2-iodoestradiol
2-methoxyestradiol-3-methyl ether
2-nitroestradiol
4-bromoestradiol
4-fluoroestradiol
4-methoxyestradiol
4-methylestradiol
estradiol-3-acetate
estradiol-3-benzoate
estradiol-3-benzyl ether
estradiol-3-carboxymethyl ether
estradiol-3-propionate
estradiol-3-sulphate
   17-One Compounds
1-methylestrone
2-fluoroestrone
2-hydroxyestrone
2-hydroxyestrone-3-methyl ether
2-methoxyestrone-3-methyl ether
3-desoxyestrone
4-hydroxyestrone
4-nitroestrone
estrone-3-acetate
estrone-3-benzoate
estrone-3-benzyl ether
estrone-3-ethyl ether
estrone-3-methyl ether
estrone-3-sulphate
   16-VARIATION
16-hydroxyestradiol-3-methyl ether
16-bromoestrone
16-bromoestradiol-3-methyl ether
16-hydroxyestradiol-3-acetate
16-bromoestradiol
16-hydroxyestradiol
2,16-dihydroxyestradiol
16-hydroxy-2-methoxyestradiol
16-hydroxy-4-methoxyestradiol
16-hydroxyestradiol-3-sulphate
   and the following compounds are commercially available from Research Plus:
   17-O Compounds
16-bromoestradiol
estradiol-3-methyl ether
estradiol-3-phosphate
2,4-dimethoxyestradiol
3,4-dibromo-2-methoxyestradiol
   17-One Compounds
2,4-dinitroestrone
estrone-3-phosphate

Aside from commercially available compounds containing suitable R¹, R², R³ and R⁴ groups, others can be readily synthesised, by existing methods, diversifying on the base estra-1,3,5(10)-triene structure. Examples of these different substituents at the 1-position (R¹) include amines, hydroxyls, ethers and alkyl chains which can be made by methods shown in references 7-10. Some examples of these different substituents at the 2-position (R²) include nitro groups, halogens, amines, hydroxyls, thiols, ethers, alkyl chains and alkylenes which can be made by methods shown in references 11-17.

Some examples of substitutions at the 3-position (R³) include hydroxyls, ethers, and esters, such as alkyl phosphates and sulphamates which can be made by methods shown in references 14 and 18-22.

Further examples of substitution at the 4-position (R⁴) include halogens, hydroxyls, ethers, alkyl groups, alkenes, cyano and nitro groups, which can be made by methods shown in references 17 and 23-26.

(B) Diversification at Z′

Benzylic oxidation of the 6-position can be performed by the use of various chromium based oxidants but most preferably chromium trioxide in acidic media. In the case of electron withdrawing substituents in the 2-position, (eg: R²=cyano) oxidation of this species may need to be performed prior to incorporation of a 2-substituent. Protection of the ketone at Z′ and possibly Z″ may be required, preferably as an acetal or, acetal derivative, to enable substitution at R² in this case. Once the ketone functionality is in place at Z′, any of the range of substituted amines can be condensed with this moiety, or alternatively, reduction with a suitable hydride reagent will give the 6-hydroxy derivative. If an oxime has been incorporated, reduction of this will lead to the amine, which itself can be further alkylated by the process of reductive amination. All of these reactions are well explored in the art [34]. The variety of reactions described above are illustrated in scheme 4 below. 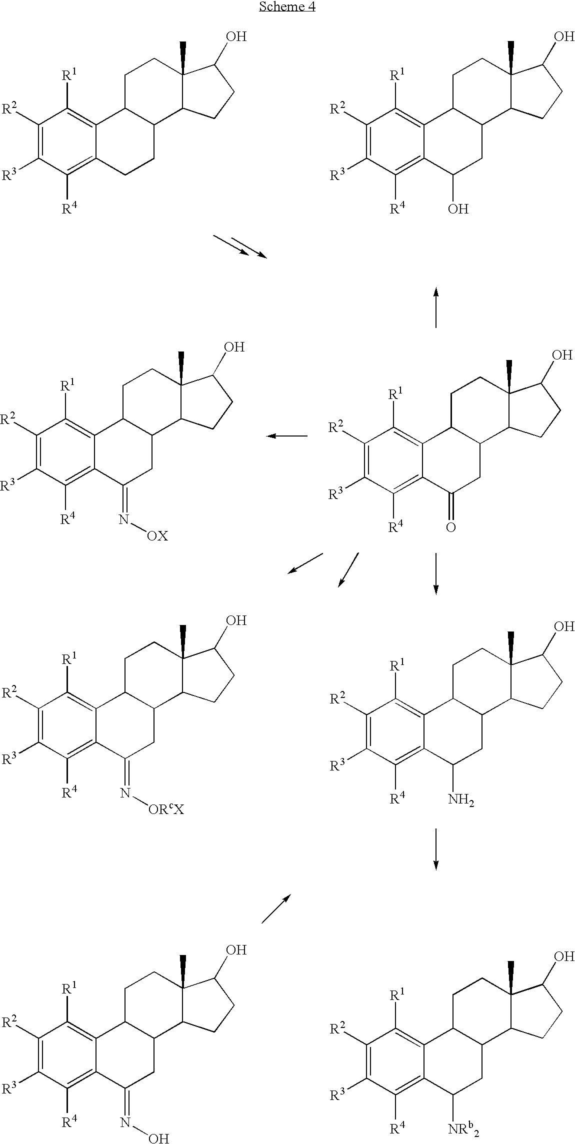
(C) Diversification at Z″

Many estra-1,3,5(10)-triene steroidal compounds having a ketone functionality already present at the 6- and/or 17-position with varied substitutions on the aromatic A ring (R¹-R⁴) are commercially available. These are immediately suitable for aryloxime formation by condensation with an appropriate substituted aryl hydroxylamine. Many commercially available estra-1,3,5(10)-triene steroidal compounds also have a 17-hydroxyl functionality present and can be readily converted to the ketone for further diversification as required at Z″. Many methods exist for oxidations of this type.[14, 27]. An important factor is the use of appropriate protecting groups (eg: acetyl, methoxyl, benzyl) to block oxidation of other potentially oxidisable groups that may be present as substituents on the aromatic ring or elsewhere on the molecule.

If a hydroxyl group is required at Z″, again a simple reduction from the ketone with a hydride reagent will give this functionality. Similarly, reductive amination or reduction of an oxime will allow for incorporation of an amine functionality at this point. Further alkylation of this amine would again be possible through reductive aminations.

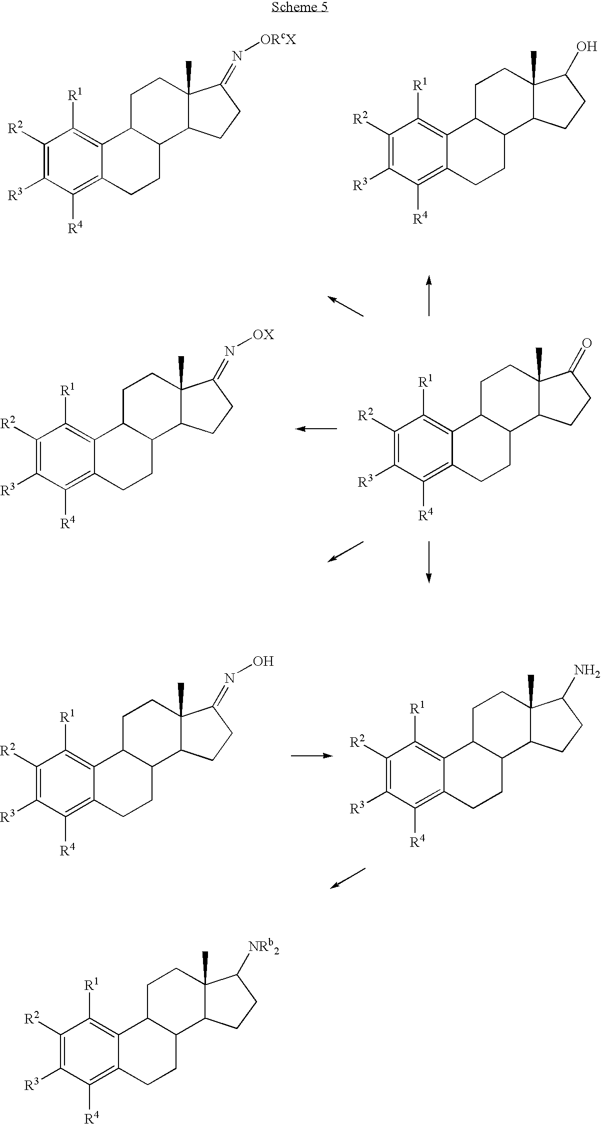

In such cases of Z′ being a ketone, oxime or aryloxime, and Z″ also being a ketone, oxime or aryloxime, Z″ should first be converted to the appropriate oxime or aryl oxime prior to oxidation of the benzylic position Z′. This situation is exemplified in the synthesis of 2-methoxy-6-(4-nitrobenzyloxy)iminoestrone-17-oxime.
(D) Diversification at the 2-Position (R²) to Introduce A.

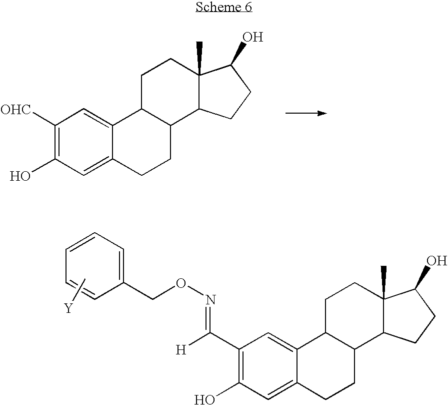

2-Formylestradiol can be prepared by the method of Pert and Ridley[23] followed by condensation with benzylhydroxylamine and 4-nitrobenzylhydroxylamine respectively to give 2-benzyloxyiminomethylestradiol and 2-(4-nitrobenzyloxy)iminomethylestradiol. For variations yielding other compounds, the aldehyde can be converted into a ketone, or the aldehyde can be located further along a hydrocarbon chain (—R^{c}—) at R².

Condensation of Substituted Arylamines with Compounds of Formulae II, III and IV

Once the required substituted arylamine (which may be a hydrazine) and precursor of formula II, III or IV have been synthesised or obtained, these are subjected to condensation by techniques known in the art, and illustrated in the examples, to yield the target compound of formula I.

Condensation reactions of the compounds of formula II, III and IV with the substituted amine are not limited to reaction with substituted aryl amine derivatives, but many other “esters” as defined above can be formed. Reaction with substituted aryl sulfonyl hydrazines, a wide variety of which are commercially available, will afford substituted aryl sulfonic acid hydrazides and likewise substituted aryl sulfonyl hydroxylamines will afford the corresponding substituted aryl sulfonyl oximes. These reactions are illustrated schematically below for the situation where the aryl group is phenyl. The phenyl group with one (or more) substituents illustrated below could be replaced with the group X, that is, a different substituted aryl.

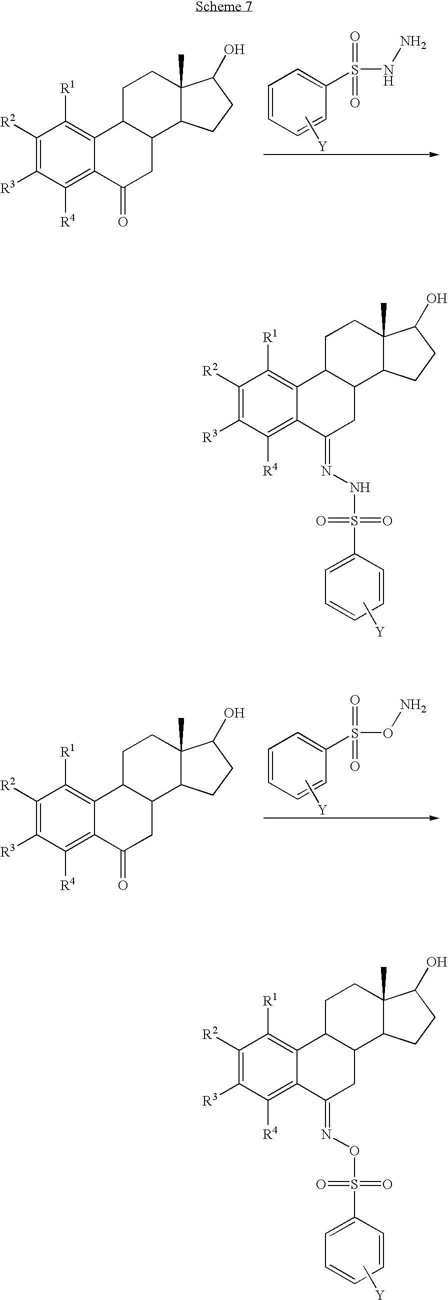

Again, condensation reactions with precursor compounds having the ketone at the 17-position are not limited to reaction with substituted aryl hydroxylamine derivatives, but many other “esters” as defined above can be formed. Reaction with substituted aryl sulfonyl hydrazines, a wide variety of which are commercially available, will afford substituted aryl sulfonic acid hydrazides and likewise substituted aryl sulfonyl hydroxylamines will afford the corresponding substituted aryl sulfonyl oximes.

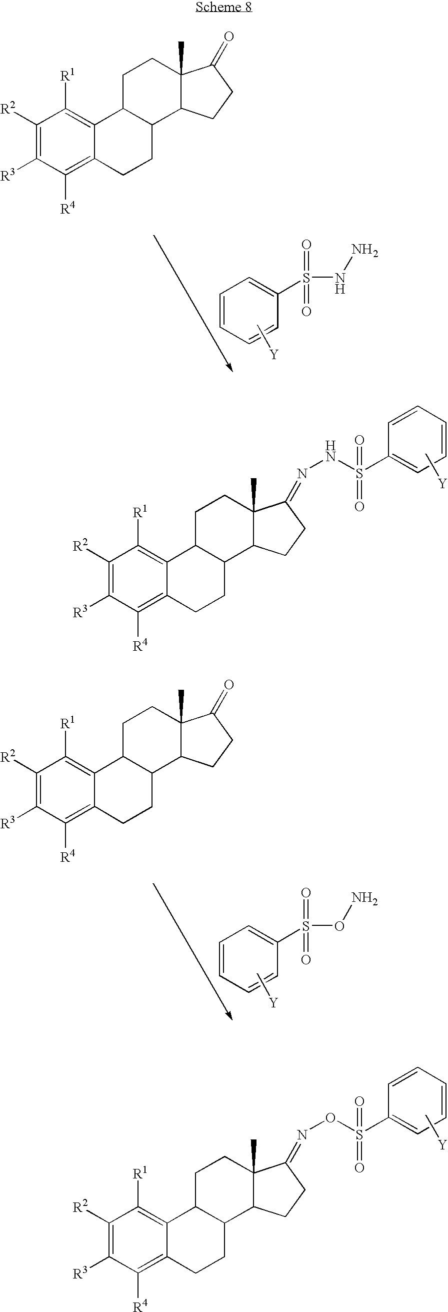

The salts of the compound of Formula I are preferably pharmaceutically acceptable, but it will be appreciated that non-pharmaceutically acceptable salts also fall within the scope of the present application, since these are useful as intermediates in the preparation of pharmaceutically acceptable salts. Examples of pharmaceutically acceptable salts include salts of pharmaceutically acceptable cations such as sodium, potassium, lithium, calcium, magnesium, ammonium and alkylammonium; acid addition salts of pharmaceutically acceptable inorganic acids such as hydrochloric, orthophosphoric, sulphuric, phosphoric, nitric, carbonic, boric, sulfamic and hydrobromic acids; or salts of pharmaceutically acceptable organic acids such as acetic, propionic, butyric, tartaric, maleic, hydroxymaleic, fumaric, citric, lactic, mucic, gluconic, benzoic, succinic, oxalic, phenylacetic, methanesulphonic, trihalomethanesulphonic, toluenesulphonic, benzenesulphonic, salicylic, sulphanilic, aspartic, glutamic, edetic, stearic, palmitic, oleic, lauric, pantothenic, tannic, ascorbic and valeric acids.

In addition, some of the compounds of the present application may form solvates with water or common organic solvents. Such solvates are encompassed within the scope of the application.

By “pharmaceutically acceptable derivative” is meant any pharmaceutically acceptable salt, hydrate or any other compound which, upon administration to the subject, is capable of providing (directly or indirectly) a compound of Formula I or an active metabolite or residue thereof.

The term “pro-drug” is used herein in its broadest sense to include those compounds which are converted in vivo to compounds of Formula I, for example, organic acid esters or ethers

The term “tautomer” is used herein in its broadest sense to include compounds of Formula I which are capable of existing in a state of equilibrium between two isomeric forms. Such compounds may differ in the bond connecting two atoms or groups and the position of these atoms or groups in the compound.

The term “isomer” is used herein in its broadest sense and includes structural, geometric and stereo isomers. As the compound of Formula I may have one or more chiral centres, it is capable of existing in enantiomeric forms. The wavy line illustrated in Formula I indicates that the substituent may be in the α or β position, or may be an isomeric mixture of these.

The compositions of the present application comprise at least one compound of Formula I together with one or more pharmaceutically acceptable carriers and optionally other therapeutic agents. Each carrier, diluent, adjuvant and/or excipient must be pharmaceutically “acceptable” in the sense of being compatible with the other ingredients of the composition and not injurious to the subject. Compositions include those suitable for oral, rectal, nasal, topical (including buccal, airway and sublingual), vaginal or parenteral (including subcutaneous, intramuscular, intravenous and intradermal) administration. The compositions may conveniently be presented in unit dosage form and may be prepared by methods well known in the art of pharmacy. Such methods include the step of bringing into association the active ingredient with the carrier which constitutes one or more accessory ingredients. In general, the compositions are prepared by uniformly and intimately bringing into association the active ingredient with liquid carriers, diluents, adjuvants and/or excipients or finely divided solid carriers or both, and then if necessary shaping the product.

The term “subject” as used herein refers to any animal having a disease or condition which requires treatment with a pharmaceutically-active agent. The subject may be a mammal, preferably a human, or may be a domestic or companion animal. While it is particularly contemplated that the compounds of the application are suitable for use in medical treatment of humans, it is also applicable to veterinary treatment, including treatment of companion animals such as dogs and cats, and domestic animals such as horses, ponies, donkeys, mules, llama, alpaca, pigs, cattle and sheep, or zoo animals such as primates, felids, canids, bovids, and ungulates. It will be understood that mechanisms controlling airway smooth muscle cell proliferation are largely conserved throughout mammals, and so it is contemplated that the compounds of the application have broad application for the treatment of conditions associated with increased airway smooth muscle cell proliferation over a range of species.

As used herein, the term “therapeutically effective amount” is meant an amount of a compound of the present application effective to yield a desired therapeutic response, for example, to prevent or treat a condition associated with airway smooth muscle cell hyperproliferation, such as asthma.

The specific “therapeutically effective amount” will, obviously, vary with such factors as the particular condition being treated, the physical condition of the subject, the type of subject being treated, the duration of the treatment, the nature of concurrent therapy (if any), and the specific formulations employed and the structure of the compound or its derivatives.

The compounds of the present application may additionally be combined with other medicaments to provide an operative combination. It is intended to include any chemically compatible combination of pharmaceutically-active agents, as long as the combination does not eliminate the activity of the compound of formula I or II. It will be appreciated that the compound of the application and the other medicament may be administered separately; sequentially or simultaneously.

Other medicaments may include, for example, one or more of the current regimen of anti-inflammatory drugs, preventer, relievers and symptom controllers where the condition is asthma.

Methods and pharmaceutical carriers for preparation of pharmaceutical compositions are well known in the art, as set out in textbooks such as Remington's Pharmaceutical Sciences, 20th Edition, Williams & Wilkins, Pennsylvania, USA.

As used herein, a “pharmaceutical carrier” is a pharmaceutically acceptable solvent, suspending agent or vehicle for delivering the compound of formula I to the subject. The carrier may be liquid or solid and is selected with the planned manner of administration in mind. Each carrier must be pharmaceutically “acceptable” in the sense of being compatible with other ingredients of the composition and non injurious to the subject.

The compound of formula I may be administered orally, topically, or parenterally in dosage unit formulations containing conventional non-toxic pharmaceutically acceptable carriers, adjuvants, and vehicles. The term parenteral as used herein includes subcutaneous injections, aerosol for administration to lungs or nasal cavity, intravenous, intramuscular, intrathecal, intracranial, injection or infusion techniques.

The present application also provides suitable topical, oral, and parenteral pharmaceutical formulations for use in the novel methods of treatment of the present application. The compounds of the present application may be administered orally as tablets, aqueous or oily suspensions, lozenges, troches, powders, granules, emulsions, capsules, syrups or elixirs. The composition for oral use may contain one or more agents selected from the group of sweetening agents, flavouring agents, colouring agents and preserving agents in order to produce pharmaceutically elegant and palatable preparations. Suitable sweeteners include sucrose, lactose, glucose, aspartame or saccharin. Suitable disintegrating agents include corn starch, methylcellulose, polyvinylpyrrolidone, xanthan gum, bentonite, alginic acid or agar. Suitable flavouring agents include peppermint oil, oil of wintergreen, cherry, orange or raspberry flavouring. Suitable preservatives include sodium benzoate, vitamin E, alphatocopherol, ascorbic acid, methyl paraben, propyl paraben or sodium bisulphite. Suitable lubricants include magnesium stearate, stearic acid, sodium oleate, sodium chloride or talc. Suitable time delay agents include glyceryl monostearate or glyceryl distearate. The tablets contain the active ingredient in admixture with non-toxic pharmaceutically acceptable excipients which are suitable for the manufacture of tablets.

These excipients may be, for example, (1) inert diluents, such as calcium carbonate, lactose, calcium phosphate or sodium phosphate; (2) granulating and disintegrating agents, such as corn starch or alginic acid; (3) binding agents, such as starch, gelatin or acacia; and (4) lubricating agents, such as magnesium stearate, stearic acid or talc. These tablets may be uncoated or coated by known techniques to delay disintegration and absorption in the gastrointestinal tract and thereby provide a sustained action over a longer period. For example, a time delay material such as glyceryl monostearate or glyceryl distearate may be employed. Coating may also be performed using techniques described in the U.S. Pat. Nos. 4,256,108; 4,160,452; and 4,265,874 to form osmotic therapeutic tablets for control release.

The compound of formula I as well as the pharmaceutically-active agent useful in the method could be administered, for in vivo application, parenterally by injection or by gradual perfusion over time independently or together. Administration may be intravenously, intraarterial, intraperitoneally, intramuscularly, subcutaneously, intracavity, transdermally or infusion by, for example, osmotic pump. For in vitro studies the agents may be added or dissolved in an appropriate biologically acceptable buffer and added to a cell or tissue. Preparations for parenteral administration include sterile aqueous or non-aqueous solutions, suspensions, and emulsions.

Generally, the terms “treating”, “treatment” and the like are used herein to mean affecting a subject, tissue or cell to obtain a desired pharmacologic and/or physiologic effect. The effect may be prophylactic in terms of completely or partially preventing a disease or sign or symptom thereof, and/or may be therapeutic in terms of a partial or complete cure of a disease. “Treating” as used herein covers any treatment of, or prevention of disease in a vertebrate, a mammal, particularly a human, and includes: (a) preventing the disease from occurring in a subject that may be predisposed to the disease, but has not yet been diagnosed as having it; (b) inhibiting the disease, i.e., arresting its development; or (c) relieving or ameliorating the effects of the disease, i.e., cause regression of the effects of the disease.

The compositions include various pharmaceutical compositions useful for ameliorating disease. The pharmaceutical compositions according to one embodiment of the application are prepared by bringing a compound of formula I, analogues, derivatives or salts thereof, or combinations of compound of formula I and one or more pharmaceutically-active agents into a form suitable for administration to a subject using carriers, excipients and additives or auxiliaries. Frequently used carriers or auxiliaries include magnesium carbonate, titanium dioxide, lactose, mannitol and other sugars, talc, milk protein, gelatin, starch, vitamins, cellulose and its derivatives, animal and vegetable oils, polyethylene glycols and solvents, such as sterile water, alcohols, glycerol and polyhydric alcohols. Preservatives include antimicrobial, anti-oxidants, chelating agents and inert gases. Other pharmaceutically acceptable carriers include aqueous solutions, non-toxic excipients, including salts, preservatives, buffers and the like, as described, for instance, in Remington's Pharmaceutical Sciences, 20th ed. Williams and Wilkins (2000) and The British National Formulary 43rd ed. (British Medical Association and Royal Pharmaceutical Society of Great Britain, 2002; http://bnf.rhn.net), the contents of which are hereby incorporated by reference. The pH and exact concentration of the various components of the pharmaceutical composition are adjusted according to routine skills in the art. See Goodman and Gilman's The Pharmacological Basis for Therapeutics (7^{th} ed., 1985) and Remington's Pharmaceutical Sciences, 20th ed. Williams and Wilkins (2000)

The pharmaceutical compositions are preferably prepared and administered in dose units. Solid dose units may be tablets, capsules and suppositories. The administration of the daily dose can be carried out both by single administration in the form of an individual dose unit or else-several smaller dose units and also by multiple administration of subdivided doses at specific intervals.

The pharmaceutical compositions may be administered locally or systemically in a therapeutically effective dose. Amounts effective for this use will, of course, depend on the severity of the disease and the weight and general state of the subject. Typically, dosages used in vitro may provide useful guidance in the amounts useful for in situ administration of the pharmaceutical composition, and animal models may be used to determine effective dosages for treatment of the cytotoxic side effects. Various considerations are described, e.g., in Langer, Science, 249: 1527, (1990).

Preferred dosage levels of the compound of formula I are of the order of about 0.1 mg to about 150 mg per kilogram body weight, more preferably of the order of 50 to 100 mg per kilogram body weight with a particularly preferred dosage of about 50 mg per kilogram body weight per day. The amount of active ingredient that may be combined with the carrier materials to produce a single dosage will vary depending upon the host treated and the particular mode of administration. For example, a formulation intended for oral administration to humans may contain about 5 mg to 1 g of an active compound with an appropriate and convenient amount of carrier material which may vary from about 5 to 95 percent of the total composition. Dosage unit forms will generally contain between from about 5 mg to 500 mg of active ingredient.

Formulations for oral use may be in the form of hard gelatin capsules wherein the active ingredient is mixed with an inert solid diluent, for example, calcium carbonate, calcium phosphate or kaolin. They may also be in the form of soft gelatin capsules wherein the active ingredient is mixed with water or an oil medium, such as peanut oil, liquid paraffin or olive oil.

Aqueous suspensions normally contain the active materials in admixture with excipients suitable for the manufacture of aqueous suspension. Such excipients may be (1) suspending agent such as sodium carboxymethyl cellulose, methyl cellulose, hydroxypropylmethylcellulose, sodium alginate, polyvinylpyrrolidone, gum tragacanth and gum acacia; (2) dispersing or wetting agents which may be (a) naturally occurring phosphatide such as lecithin; (b) a condensation product of an alkylene oxide with a fatty acid, for example, polyoxyethylene stearate; (c) a condensation product of ethylene oxide with a long chain aliphatic alcohol, for example, heptadecaethylenoxycetanol; (d) a condensation product of ethylene oxide with a partial ester derived from a fatty acid and hexitol such as polyoxyethylene sorbitol monooleate, or (e) a condensation product of ethylene oxide with a partial ester derived from fatty acids and hexitol anhydrides, for example polyoxyethylene sorbitan monooleate.

Compounds of formula I may also be administered in the form of liposome delivery systems, such as small unilamellar vesicles, large unilamellar vesicles, and multilamellar vesicles. Liposomes can be formed from a variety of phospholipids, such as cholesterol, stearylamine, or phosphatidylcholines.

### EXAMPLES

The invention will now be described in detail by way of reference only to the following non-limiting examples.

Preparation of Candidate Compounds of Formula I

### Example 1

### Synthesis of 2-methoxy-6-(4′-nitrobenzyl)oxyiminoestradiol (“4NO”)

### Example 1a

2-Methoxyestradiol (1) was diacetylated by treatment with acetic anhydride in pyridine in 95% yield. Benzylic oxidation of the bis-protected species was effected with chromium trioxide in an acetic acid/water mixture in 45% yield. Both acetates were removed by treatment of the newly formed keto compound (3) with potassium carbonate in aqueous methanol in 98% yield. Condensation of this ketone was then effected with O-(4-nitrobenzyl) hydroxylamine in methanol to give 2-methoxy-6-(4-nitrobenzyloxy)iminoestradiol (5) in 99% yield.

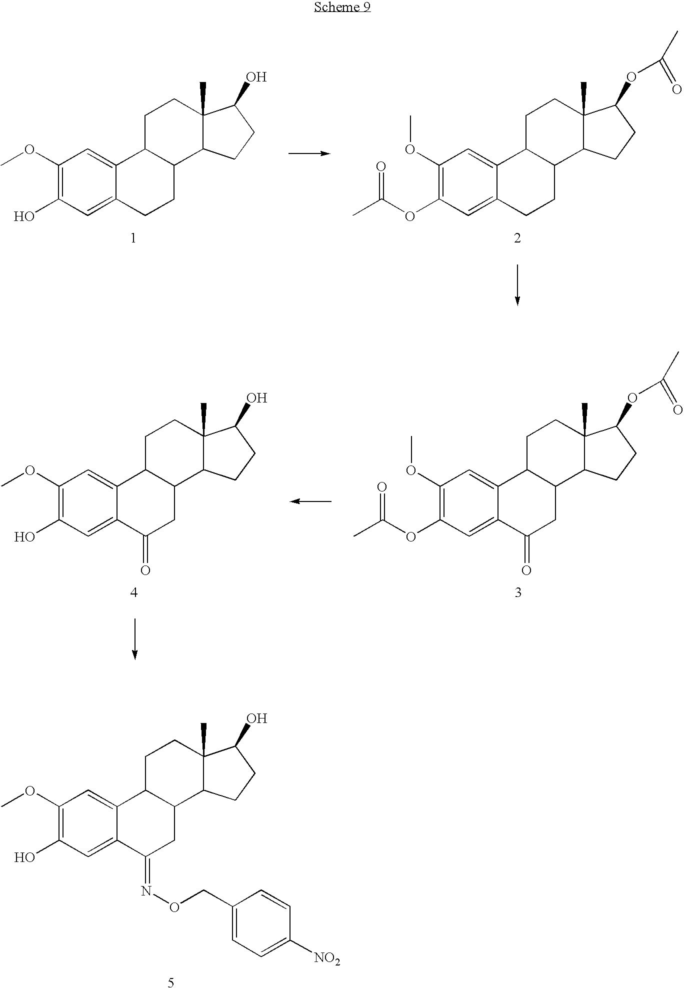

### Example 1b

### 3,17-Bis-acetyloxy-2-methoxyestra-1,3,5(10)triene (2)

2-Methoxyestradiol (127.5 mg, 0.426 mmol) was dissolved in anhydrous pyridine (6.0 mL) under a nitrogen atmosphere and cooled to 0° C. Acetic anhydride (3.0 mL) was added dropwise and the reaction allowed to warm to room temperature. After overnight stirring, the reaction mixture was cooled to 0° C. at which time 50.0 mL of 1M aqueous hydrochloric acid was added and the reaction allowed to warm to room temperature. Extraction was performed with ethyl acetate (3×50 mL) and the combined organic extracts were sequentially washed with 3M aqueous hydrochloric acid (50 mL), water (50 mL) and brine (50 mL). The organic layers were dried over sodium sulfate then filtered and the solvent evaporated in vacuo to give a white solid (154 mg). The product was homogeneous when analysed by TLC therefore purification was deemed unnecessary, however an analytical sample was purified on a silica gel column using 1:5 ethyl acetate/petroleum spirit as eluent.

R_{f} 0.88 [ethyl acetate-petroleum spirit (1:1), silica gel]; ¹H NMR (CDCl₃): δ6.86 (s, 1H), 6.71 (s, 1H), 4.67 (t, J=8.3 Hz, 1H), 3.78 (s, 3H), 2.77-2.74 (m, 2H), 2.28 (s, 3H), 2.04 (s, 3H), 2.26-1.23 (m, 13H), 0.81 (s, 3H)

### Example 1c

### 3,17-Bis-acetyloxy-2-methoxy-6-oxoestra-1,3,5(10)triene (3)

Chromium trioxide (79.8 mg, 0.798 mmol) was dissolved in 2.0 mL of 90% (v/v) aqueous acetic acid with stirring over a 90 minute period. This oxidizing mixture was added dropwise at 10° C. to a stirred solution of 3,17-bis-acetyloxy-2-methoxyestra-1,3,5(10)triene (2) (72.5 mg, 0.188 mmol). The mixture was stirred at 10° C. for 30 minutes at which time the reaction was poured onto an ice/water mixture (30 mL) which was extracted with ethyl acetate (3×15 mL). The combined extracts were washed with water (20 mL), saturated aqueous sodium bicarbonate solution (20 mL), water (20 mL), and brine (20 mL) prior to drying over sodium sulfate and filtration. Evaporation of the organic solvent yielded a solid yellow residue. This residue was purified by flash chromatography on silica gel using a 1:3 ethyl acetate/petroleum spirit mixture as eluent to give a white solid (34 mg, 45%).

R_{f} 0.36[ethyl acetate-petroleum spirit (1:3), silica gel]; ¹H NMR (CDCl₃): δ7.73 (s, 1H), 6.92 (s, 1H), 4.71 (t, J=8.5 Hz, 1H), 3.90 (s, 3H), 2.69 (dd, J=16.9, 3.4 Hz, 1H), 2.53 (dt, J=16.9, 3.4 Hz, 1H), 2.31 (s, 3H), 2.06 (s, 3H), 2.40-1.34 (m, 13H), 0.83 (s, 3H); ¹³C NMR (CDCl₃): δ195.81, 171.10, 168.92, 155.40, 146.95, 138.42, 126.06, 121.79, 108.36, 82.09, 55.97, 49.74, 43.49, 43.26, 42.63, 39.57, 36.38, 27.36, 25.35, 22.92, 21.12, 20.52, 11.84.

### Example 1d

### 2-methoxy-6-oxo-estradiol (4)

Anhydrous potassium carbonate (20 mg, 0.145 mmol) was added to a stirred solution of 3,17-bis-acetyloxy-2-methoxy-6-oxoestra-1,3,5(10)triene (3)(7) (34 mg, 0.085 mmol) in 8.0 mL of methanol under a nitrogen atmosphere. Water (3.0 mL) was added and reaction stirred at room temperature for 16 hours. The pH of the reaction was then adjusted to pH 4 with dropwise addition of 1M hydrochloric acid aqueous solution. Extraction was performed with ethyl acetate (3×10 mL) then the combined layers were washed with water (10 mL) and brine solution (10 mL) prior to drying over sodium sulfate and filtration. Evaporation of the solvent in vacuo yielded a cream coloured solid which was purified by flash chromatography on silica:gel (3:2 ethyl acetate: petroleum spirit) to give 26.4 mg (98%) of the target compound as a white crystalline solid. mp 189-190° C. (dec); R_{f} 0.34[ethyl acetate-petroleum spirit (3:2), silica gel]; ¹H NMR (CD₃OD): δ7.36 (s, 1H), 6.91 (s, 1H), 3.92 (s, 3H), 3.66 (t, J=8.5 Hz, 1H), 2.52 (dd, J=16.8, 3.5 Hz, 1H), 2.43-1.26 (m, 12H), 0.76 (s, 3H).

### Example 1e

### 2-Methoxy-6-(4-nitrobenzyloxy)iminoestradiol (“4NO”) (5)

To a stirred solution of 2-methoxy-6-oxo-estradiol (4) (176.0 mg, 0.556 mmol) in methanol (25.0 mL) was added 4-nitrobenzylhydroxylamine hydrochloride (266 mg, 1.574 mmol) under a nitrogen atmosphere. To this solution was added 4-Polyvinylpyridine (25% cross-linked, 1.033 g) and the reaction mixture heated to reflux. After 17 hours, the reaction was cooled to room temperature and the solvent removed in vacuo. The crude product was dissolved in anhydrous tetrahydrofuran and treated with PS-isocyanate (loading 1.25 mmol/g, 2.522 g) resin for 15.5 hours. Filtration of the mixture through a celite pad and further elution with tetrahydrofuran (4×50 mL) gave a pale yellow solution that was evaporated in vacuo to yield an amorphous solid (257 mg, 99%).

R_{f} 0.60[ethyl acetate-petroleum spirit (2:1), silica gel]; ¹H NMR (CDCl₃): δ8.21 (d, 8.8 Hz, 2H), 7.53 (d, 8.8 Hz, 2H), 7.46 (s, 1H), 6.78 (s, 1H), 5.49 (s, 1H), 5.27 (s, 2H), 3.90 (s, 3H), 3.75 (t, J=8.6 Hz, 1H) 3.14 (dd, J=18.1, 4.4 Hz, 1H), 2.19-1.22 (m, 12H), 0.77 (s, 3H); ESIMS-[M+H]⁺ m/z=467; HRESI-MS: [M+H]⁺ 467.2185 (467.2182 calc.)

### Example 2

### Synthesis of 2-methoxy-6-(4-nitrobenzyloxy)iminoestrone-17-oxime (“4NOM”) (10)

### Example 2a

2-Methoxyestrone was condensed with excess hydroxylamine in methanol at reflux to afford 2-methoxyestrone-17-oxime (6) as a single geometrical isomer. Without purification this compound was treated with acetic anhydride in pyridine to afford the 3,17-diacetylated oxime derivative (7) in 90% yield over the two step process. Benzylic oxidation of the acetyl protected species with chromium trioxide in acetic acid/water mixture affords the 6-oxo derivative which is immediately deprotected to give 2-methoxy-6-oxo-estrone-17-oxime (9) in 71% yield over this two step process. Treatment of the ketone with 4-nitrobenzylhydroxylamine in methanol affords 2-methoxy-6-(4-nitrobenzyloxy)iminoestrone-17-oxime (10) in 33% yield.

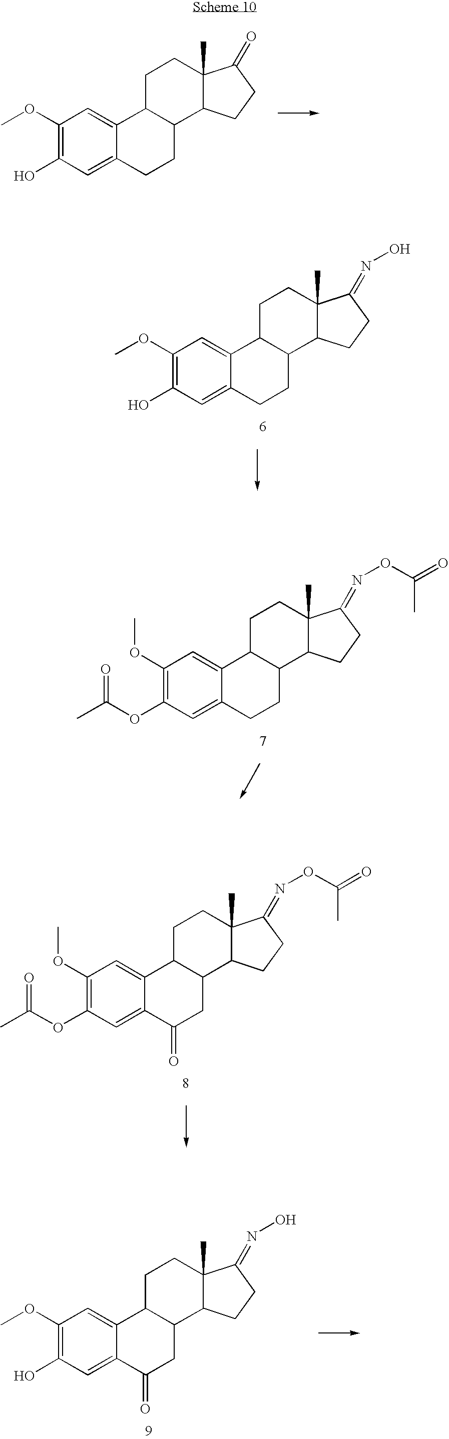

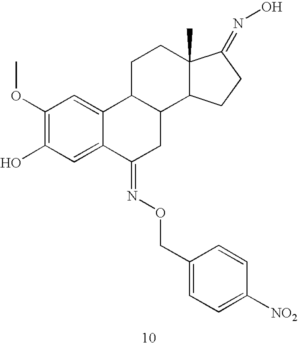

### Example 2b

### 2-Methoxyestrone-17-oxime (6)

To a suspension of 2-methoxyestrone (1.698 g, 5.651 mmol) in anhydrous methanol (160.0 mL) under a nitrogen atmosphere was added hydroxylamine hydrochloride (1.110g, 15.97 mmol), with complete dissolution being achieved with gentle heating of the mixture to 35° C. 4-Polyvinylpyridine (8.00 g, 25% cross-linked) was added and the reaction brought to reflux. After 14.5 hours the reaction was cooled to room temperature and the solvent removed in vacuo. The residue was suspended in anhydrous tetrahydrofuran (60.0 mL) and filtered through a sintered glass funnel. The resin was further washed with tetrahydrofuran (40.0 mL, 50.0 mL) and the filtrate evaporated in vacuo to yield the crude product as a white foam. (1.942 g).

R_{f} 0.28[ethyl acetate-petroleum spirit (1:2), silica gel]; ¹H NMR (CDCl₃): δ 6.79 (s, 1H), 6.65 (s, 1H), 5.50 (s, 1H), 3.86 (s, 3H), 2.90-2.70 (m, 2H), 2.60-1.32 (m, 13H), 0.96 (s, 3H); ESIMS-[M+H]⁺ m/z=316

### Example 2c

### 3-Acetyloxy-2-methoxyestra-1,3,5(10)trien-17-one acetyloxime (7)

Crude 2-methoxyestrone-17-oxime (6) (1.937 g) was dissolved in pyridine (12.0 mL, 148.8 mmol) under a nitrogen atmosphere. Acetic anhydride (6.0 mL, 63.6 mmol) was added dropwise with stirring. Stirring was continued at room temperature for 16 hours at which time the reaction was quenched by careful addition of aqueous 1M ammonium chloride solution (50.0 mL, 50.0 mmol). The reaction mixture was then extracted with ethyl acetate (100 mL then 2×50 mL) and the combined organic layers washed with 1M ammonium chloride solution (3×50 mL), 1M hydrochloric acid aqueous solution (50 mL), water (50 mL) and brine (50 mL). The organic phase was dried over anhydrous sodium sulfate, filtered and evaporated in vacuo then dried under high vacuum to yield an off white solid (2.099 g) representing a yield of 90% over the two steps.

R_{f} 0.38[ethyl acetate-petroleum spirit (1:2), silica gel]; ¹H NMR (CDCl₃): δ6.89 (s, 1H), 6.75 (s, 1H), 3.81 (s, 3H), 2.84-2.72 (m, 2H), 2.60-1.32 (m, 13H), 2.31 (s, 3H), 2.16 (s, 3H) 1.02 (s, 3H).

### Example 2d

### 3-Acetyloxy-2-methoxy-6-oxoestra-1,3,5(10)trien-17-one acetyloxime (8)

Chromium trioxide (2.26 g, 22.6 mmol) was dissolved in 50.0 mL of 90% (v/v) aqueous acetic acid with shaking over a 60 minute period. This oxidizing mixture was added dropwise at 5-8° C. to a stirred solution of 3-acetyloxy-2-methoxyestra-1,3,5(10)trien-17-one acetyloxime (7) (2.099 g, 5.07 mmol). The mixture was stirred at 5-8° C. for 42 minutes at which time the reaction was quenched by addition of water (150 mL). The reaction mixture was then extracted with ethyl acetate (2×150 mL, then 100 mL) and the combined extracts washed with saturated aqueous sodium bicarbonate solution (3×100 mL), water (100 mL), and brine (100 mL). The organic phase was then dried over sodium sulfate and filtered through a celite pad which was further washed with ethyl acetate (2×20 mL). Evaporation of the solvent yielded a pale yellow solid. The product was homogeneous when analysed by TLC, therefore purification was deemed unnecessary, however an analytical sample was purified on a silica gel column using 1:1 ethyl acetate/petroleum spirit as eluent.

R_{f} 0.28[ethyl acetate-petroleum spirit (1:1), silica gel]; ¹H NMR (CDCl₃): δ7.77 (s, 1H), 6.95 (s, 1H), 3.93 (s, 3H), 2.69 (dd, J=16.9, 3.4 Hz, 1H), 2.53 (dt, J=16.9, 3.4 Hz, 1H), 2.31 (s, 3H), 2.06 (s, 3H), 2.40-1.34 (m, 13H), 1.05 (s, 3H); ESIMS -[M+H]⁺ m/z=414.

### Example 2e

### 2-methoxy-6-oxo-estrone-17-oxime (9)

Anhydrous potassium carbonate (12.56 g, 90.87 mmol) was added to a stirred solution of the crude 3-acetyloxy-2-methoxy-6-oxoestra-1,3,5(10)trien-17-one acetyloxime (8) in methanol (150 mL) under a nitrogen atmosphere. After 13.5 hours, water (5.0 mL) was added and reaction stirred at 40° C. for 12 hours. The reaction mixture was then diluted with water (150 mL) and the pH of the solution was adjusted to pH 7 by dropwise addition of 1M hydrochloric acid aqueous solution. Extraction was performed with ethyl acetate (3×150 mL), then the combined layers were washed with water (150 mL) and brine (150 mL) prior to drying over sodium sulfate and filtration. Evaporation of the solvent in vacuo yielded a cream coloured solid which was purified by flash chromatography on silica gel (2:1 ethyl acetate: petroleum spirit) to give 1.233 g (71% over two steps) of an off white solid.

R_{f} 0.46 [ethyl acetate-petroleum spirit (2:1), silica gel]; ¹H NMR (CDCl₃): δ7.61 (s, 1H), 6.85 (s, 1H), 5.57 (s, 1H), 3.97 (s, 3H), 2.77 (dd, J=16.6, 3.4 Hz, 1H), 2.61-1.41 (m, 12H), 0.97 (s, 3H); ESIMS -[M+H]⁺ m/z=330

### Example 2f

### 2-Methoxy-6-(4-nitrobenzyloxy)iminoestrone-17-oxime (“4NOM”) (10)

To a stirred solution of 2-methoxy-6-oxo-estrone-17-oxime (9)(22.8 mg, 0.069 mmol) in anhydrous methanol (10.0 mL) under a nitrogen atmosphere was added 4-nitrobenzylhydroxylamine hydrochloride (42.5 mg, 0.208 mmol) and 4-polyvinylpyridine (185.0 mg, 25% cross-linked). The reaction was stirred at room temperature for 24 hours at which time the solvent was evaporated in vacuo. The pale yellow residue was suspended in anhydrous tetrahydrofuran (15.0 mL) and excess nucleophile scavenged with PS-benzaldehyde resin (177.0 mg) over a 24 hour period at room temperature. Filtration of the solids on a solid phase extraction cartridge (Supelco LC-CN) and washing with further tetrahydrofuran (4×5.0 mL) followed by removal of solvent under a stream of nitrogen gave a pale yellow oil that was further purified by flash chromatography on silica:gel (2:1 ethyl acetate: petroleum spirit) to give 11.2 mg (33%) of the required product as a pale yellow solid.

R_{f} 0.33[ethyl acetate-petroleum spirit (3:2), silica gel]; ESIMS-[M+H]⁺ m/z=480

¹H NMR (CDCl₃): δ8.21 (d, J=8.7 Hz, 2H), 7.54 (d, J=8.7 Hz, 2H), 7.48 (s, 1H), 6.78 (s, 1H), 5.5.5 (br s, 1H), 5.27 (s, 2H), 3.91 (s, 3H), 3.22 (dd, J=18.1, 4.4 Hz, 1H), 2.65-1.21 (m, 12H), 0.94 (s, 3H); ¹³C NMR (CDCl₃) δ170.63, 154.16, 148.08, 147.41, 146.09, 143.97, 135.09, 128.34, 123.58, 123.27, 109.89, 106.83, 74.67, 55.87, 53.20, 43.97, 41.84, 36.50, 33.56, 29.43, 25.55, 25.07, 22.91, 17.01; ESI-MS (20v) m/z 480 (100) [M+H]⁺; HRESI-MS: [M+H]⁺ 480.2125 (480.2135 calc.);

### Example 3

### Synthesis of 2-benzyloxyiminomethylestradiol and 2-(4-nitrobenzyloxy)iminomethylestradiol

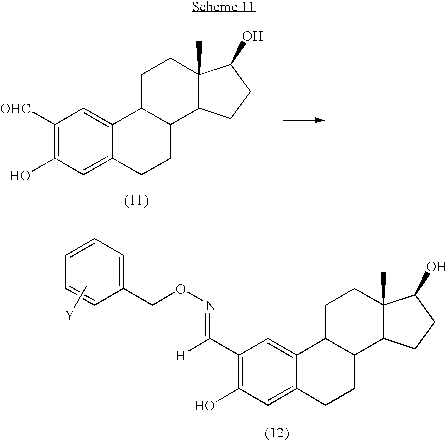

2-Formylestradiol was prepared by the method of Pert and Ridley[23] followed by condensation with benzylhydroxylamine and 4-nitrobenzylhydroxylamine respectively to give 2-benzyloxyiminomethylestradiol (Y═H; 12a) and 2-(4-nitrobenzyloxy)iminomethylestradiol (Y═NO₂; 12b).

Two solutions of 2-formylestradiol (7.2 mg, 0.190 mmol) in 5 mL of anhydrous THF were stirred under a nitrogen atmosphere and O-aryl hydroxylamines (as their hydrochloride salts) were added to each as indicated below.

TABLE 1

| | Quantity | Molar |
|---|---|---|
| Hydroxylamine | (mg) | Amount(mmol) |
| O-Benzylhydroxylamine•HCl | 11.5 | 0.719 |
| O-4-Nitrobenzylhydroxylamine•HCl | 14.7 | 0.719 |

Upon addition of the respective hydroxylamines, a quantity of Poly(4-vinylpyridine), 25% cross linked (200-220 mg) was added to each reaction tube with each tube being heated to reflux and stirred for sixteen hours. The reactions were allowed to cool to room temperature and PS-Benzaldehyde scavenger resin (32-42 mg, loading 1.31 mmol/g) was added to each tube. Reactions were stirred for 2 hours at which time all residues were removed by filtration through a polyethylene frit. The residues were washed with THF (3×5 mL) with residual solvent being removed under a stream of nitrogen.

The yielded products were analysed by thin layer chromatography (SiO2, 1:1; Ethyl Acetate: Petroleum Spirit), high performance liquid chromatography; gradient elution 70% CH₃CN/H₂O—98% CH₃CN /H₂O over 22 minutes (Phenomenex C18 column spherex 5, (250×4.6 mm)), and electrospray mass spectrometry. The results are shown in the following table.

TABLE 2

| | | HPLC | HPLC | |
|---|---|---|---|---|
| | | Retention | Purity | |
| | ESI-MS | Time | (%) at | TLC |
| Compound | [M + H]⁺ | (min) | 220 nm | Rf |
| 2- | 406 | 19.2 | 93.0 | 0.70 |
| benzyloxyiminomethyl- | | | | |
| estradiol | | | | |
| 2-(4- | 451 | 19.4 | 94.3 | 0.57 |
| nitrobenzyloxy)imino- | | | | |
| methylestradiol | | | | |

### Example 4

### Preparation of 6-(3,5-difluorobenzyloxy)imino-2-methoxyestrone-17-oxime CP-DM-4-36)

This is prepared via two intermediates, as follows.

### Example 4a

### Preparation of N-(3,5-Difluorobenzyloxy)phthalimide

To a solution of N-hydroxyphthalimide (reagent A) (592.3 mg, 3.631 mmol) in 25 mL anhydrous tetrahydrofuran under a nitrogen atmosphere was added 3,5-difluorobenzyl bromide (reagent B) (516.7 μL, 3.994 mmol). N,N-diisopropylethylamine (reagent C) (1.367 mL, 7.988 mmol) was then added dropwise with stirring to effect an immediate colour change. The reaction was then heated to reflux and held at this temperature for 20 hours before cooling to ambient temperature causing formation of a thick white precipitate. The reaction mixture was then diluted with 25 mL of water prior to extraction with 5×25 mL ethyl acetate. Combined organic fractions were washed with water (25 mL) and brine (25 mL) prior to drying over anhydrous sodium sulphate. Evaporation in vacuo gave 936.1 mg of the product (D) (89%) as a white solid.

R_{f} 0.375[dichloromethane, silica gel]; ¹H NMR (CDCl₃): δ7.86-7.75 (m, 4H), 7.11-7.09 (m, 2H), 6.85-6.79 (m, 1H), 5.17 (s, 2H). ESI-MS (40V) m/z 312 (100) [M+Na]⁺, 601 (34) [2M+Na]⁺.

### Example 4b

### Preparation of O-(3,5-difluorobenzyl)hydroxylamine hydrochloride

N-(3,5-difluorobenzyloxy)phthalimide (D) (143.0 mg, 0.494 mmol) was dissolved in 5 mL glacial acetic acid and hydrochloric acid 37% (1.00 mL, 31.8 mmol) added dropwise with stirring under a nitrogen atmosphere. The reaction was heated to reflux and held at that temperature for 3 hours before cooling to ambient temperature. The reaction mixture was evaporated under a stream of nitrogen prior to suspension in 0.2 M aqueous sodium hydroxide solution. The pH of the mixture was further adjusted to pH 8<10 with dropwise addition of 6 M sodium hydroxide aqueous solution. Extraction was performed with 3×15 mL ethyl acetate. Combined organic extracts were dried over anhydrous sodium sulphate and evaporated to give a pale yellow oil that was dissolved in ethanol (1 mL) prior to dropwise addition of 1 M hydrogen chloride in diethyl ether (3 mL) which immediately gave a white precipitate. Addition of further diethyl ether (25 mL) allowed centrifugation, decanting, further washing with diethyl ether (25 mL), further centrifugation and decanting prior to air drying which yielded a white solid (E) (78.6 mg, 82%).

¹H NMR (DMSO-d₆): δ10.92 (br s, 3H), 7.24 (tt, J=9.6, 2.4 Hz, 1H), 7.17-7.09 (m, 2H), 4.96 (s, 2H); ESI-MS (20V) m/z 160 (100) [M+H]⁺, 143 (43) [M+H—NH₂]⁺.

### Example 4c

### Preparation of 6-(3,5-difluorobenzyloxy)imino-2-methoxyestrone-17-oxime

### (CP-DM-4-36)

2-methoxy-6-oxo-estrone-17-oxime (F) (15.0 mg, 0.0455 mol), O-(3,5-difluorobenzyl)hydroxylamine hydrochloride (E) (11.6 mg, 0.059 mmol) and 4-polyvinylpyridine (G) (25% cross-linked, 200 mg) were all added to reaction vessel under a nitrogen atmosphere and solvated with methanol (10 mL) with stirring at ambient temperature. Reaction proceeded at ambient temperature for 64 hours at which time the reaction mixture was evaporated in vacuo. The resulting residue was suspended in anhydrous tetrahydrofuran (10 mL) and treated with PS-Benzaldehyde resin (H) (46 mg, 1.20 mmolg⁻¹). The suspension was filtered through a LC-C18 solid phase extraction cartridge which was further washed with 3×5 mL THF. Evaporation, followed by flash chromatography on silica gel [ethyl acetate-petroleum spirit (40-60° C.) (1:1)] gave a white amorphous solid (20.2 mg, 94%).

¹H NMR (CDCl₃): δ7.50 (s, 1H), 6.89-6.82 (m, 2H), 6.78 (s, 1H), 6.76-6.64 (m, 1H), 5.15 (s, 2H), 3.91 (s, 3H), 3.22 (dd, J=18.0, 4.5 Hz, 1H, 2.62-1.20 (m, 12H), 0.94 (s, 3H); ESI-MS (20v) m/z 471 (100) [M+H]⁺; HRESI-MS: [M+H]⁺ 471.2086 (471.2095 calc.)

### Example 5

### Preparation of 6-(3,5-difluorobenzyloxy)imino-2-methoxyestradiol CP-DM-4-35)

The same procedure was used as outlined in Example 4c above with the following variations.

As reagent F, 2-methoxy-6-oxo-estradiol was used in an amount of 15.1 mg (0.048 mmol), reagent E, O-(3,5-difluorobenzyl)hydroxylamine hydrochloride was used in an amount of 12.1 mg (0.062 mmol), resin G was used in an amount of 210 mg, and resin H was used in an amount of 43.0 mg. The reaction was conducted for 64 hours. The product, 2-methoxy-6-(3,5-difluorobenzyloxy)iminoestradiol, was obtained in a yield of 88%.

¹H NMR (CDCl₃): δ7.49 (s, 1H), 6.89-6.87 (m, 2H), 6.78 (s, 1H), 6.74-6.68 (m, 1H), 5.50 (br s, 1H), 5.15 (s, 2H), 3.90 (s, 3H), 3.75 (t, J=8.5 Hz, 1H), 3.14 (dd, J=18.0, 4.5 Hz, 1H), 2.62-1.22 (m, 12H), 0.94 (s, 3H); ¹³C NMR (CDCl₃) δ 164.04 (dd, J=248.0, 13.0 Hz), 155.41, 149.02, 144.93 (t, J=9.9 Hz), 136.51, 124.55, 111.32 (dd, J=18.7, 6.5 Hz), 110.95, 107.92, 107.90, 103.86 (t, J=25.2 Hz), 82.69, 75.71, 56.90, 51.53, 44.09, 42.88, 38.30, 37.26, 31.63, 30.61, 26.76, 24.14, 12.00; ESI-MS (20v) m/z 458 (100) [M+H]⁺; HRESI-MS: [M+H]⁺ 458.2131 (458.2143 calc.)

### Example 6

### Preparation of 6-(3-methoxybenzyloxy)imino-2-methoxyestrone-17-oxime CP(DM-4-66)

This is prepared via two intermediates, as follows.

### Example 6a

### Preparation of N-(3-methoxybenzyloxy)phthalimide

The same procedure was used as outlined in Example 4a above with the following variations.

As reagent B, 3-methoxybenzyl bromide was used in 486.1 μL (3.47 mmol), reagent A was used in an amount of 514.8 mg (3.16 mmol), and 1.20 mL (7.01 mmol) of reagent C was used. The reaction was conducted for 15 hours at reflux. Extraction was with 1×15 mL ethyl acetate and 2×25 mL dichloromethane. The product D, N-(3-methoxybenzyloxy)phthalimide, was obtained in a yield of 95%.

¹H NMR (CDCl₃): δ7.84-7.70 (m, 4H), 7.26 (m, 1H), 7.10-7.06 (m, 2H), 6.89 (dd, J=8.3, 2.8 Hz, 1H), 5.17 (s, 2H), 3.80 (s, 3H).

### Example 6b

### Preparation of O-(3-methoxybenzyl)hydroxylamine hydrochloride

The same procedure was used as outlined in Example 4b above with the following variations.

As reagent D, N-(3-methoxybenzyloxy)phthalimide was used in 211.5 mg (0.791 mmol), glacial acetic acid was used in a 10 mL quantity and hydrochloric acid 37% was used in a 2.00 mL (63.6 mmol) quantity. The reaction was conducted for 4.5 hours at reflux. The product E, O-(3-methoxybenzyl)hydroxylamine hydrochloride, was obtained in a yield of 92%.

¹H NMR (DMSO-d6): δ10.15 (br s, 3H), 7.32 (t, J=7.5 Hz, 1H), 6.97-6.92 (m, 3H), 4.88 (s, 2H), 3.76 (s, 3H).

### Example 6c

### Preparation of 6-(3-methoxybenzyloxy)imino-2-methoxyestrone-17-oxime (CP-DM-4-66)

The same procedure was used as outlined in Example 4c above with the following variations.

As reagent F, 2-methoxy-6-oxo-estrone-17-oxime was used in an amount of 13.0 mg (0.040 mmol), reagent E from Example 6b was used in an amount of 9.7 mg (0.051 mmol), resin G was used in an amount of 103.9 mg, and resin H was used in an amount of 35.0 mg. The reaction was conducted for 64 hours. Filtration was through a LC-CN solid phase extraction cartridge. The product, 2-methoxy-6-(3-methoxybenzyloxy)iminoestrone-17-oxime, was obtained in a yield of 45%.

¹H NMR (CDCl₃): δ7.55 (s, 1H), 7.26 (m, 1H), 7.15 (br s, 1H), 7.01-6.97 (m, 2H), 6.84 (dd, J=8.1, 2.6 Hz, 1H) 6.77 (s, 1H), 5.49 (br s, 1H), 5.17 (s, 2H), 3.91 (s, 3H), 3.82 (s, 3H), 3.20 (dd, J=18.0, 4.5 Hz, 1H), 2.63-1.24 (m, 12H), 0.76 (s, 3H); ESI-MS (20v) m/z 465 (100) [M+H]; HRESI-MS; [M+H]⁺ 465.2385 (465.2389 calc.)

### Example 7

### Preparation of 6-(3-methoxybenzyloxy)imino-2-methoxyestradiol (CP-DM-4-62)

The same procedure was used as outlined in Example 4c above with the following variations.

As reagent F, 2-methoxy-6-oxo-estradiol was used in an amount of 11.2 mg (0.035 mmol), reagent E from Example 6b was used in an amount of 8.7 mg (0.046 mmol), resin G was used in an amount of 120.2 mg, and resin H was used in an amount of 35 mg. The reaction was conducted for 64 hours. Filtration was through a LC-CN solid phase extraction cartridge. The product, 2-methoxy-6-(3-methoxybenzyloxy)iminoestradiol, was obtained in a yield of 70%.

¹H NMR (CDCl₃): δ7.54 (s, 1H), 7.26 (m, 1H), 6.97 (m, 2H), 6.84 (dd, J=8.4, 2.6 Hz, 1H) 6.77 (s, 1H), 5.49 (br s, 1H), 5.11 (s, 2H), 3.90 (s, 3H), 3.81 (s, 3H), 3.73 (t, J=8.5 Hz, 1H), 3.13 (dd, J=18.0, 4.4 Hz, 1H), 2.33-1.15 (m, 12H), 0.76 (s, 3H); ¹³C NMR (CDCl₃) δ159.59, 153.86, 147.88, 143.88, 139.94, 135.37, 129.32, 123.76, 120.41, 113.60, 113.22, 109.99, 106.84, 81.64, 76.06, 55.82, 55.21, 50.50, 43.02, 41.83, 37.27, 36.22, 30.57, 29.58, 25.71, 23.08, 10.94; ESI-MS (20v) m/z 452 (100) [M+H]⁺; HRESI-MS: [M+H]⁺ 452.2432 (452.2437 calc.)

### Example 8

### Preparation of 6-(3-trifluoromethoxybenzyloxy)imino-2-methoxyestrone-17-oxime (CP-DM-4-67)

This was prepared via 2 intermediates, in stages corresponding to those set out in Example 4.

### Example 8a

### Preparation of N-(3-trifluoromethoxybenzyloxy)phthalimide

The same procedure was used as outlined in Example 4a above with the following variations.

As reagent B 3-trifluormethoxybenzyl bromide was used in 546.0 μL (3.37 mmol), reagent A was used in an amount of 499.1 mg (3.16 mmol), and 1.20 mL (7.01 mmol) of reagent C was used. The reaction was conducted for 15 hours at reflux. Extraction was with 1×15 mL ethyl acetate and 2×25 mL dichloromethane. The product D, N-(3-trifluoromethoxybenzyloxy)phthalimide, was obtained in a yield of 94%.

¹H NMR (CDCl₃): δ7.82-7.71 (m, 4H), 7.47 (d, J=7.7 Hz, 1H), 7.42-7.38 (m, 2H), 7.22-7.20 (m, 1H), 5.19 (s, 2H).

### Example 8b

### Preparation of O-(3-trifluoromethoxybenzyl)hydroxylamine hydrochloride

The same procedure was used as outlined in Example 4b above with the following variations.

As reagent D, N-(3-trifluoromethoxybenzyloxy)phthalimide was used in 202.8 mg (0.631 mmol), glacial acetic acid was used in a 10 mL quantity and hydrochloric acid 37% was used in a 2.00 mL (63.6 mmol) quantity. The reaction was conducted for 4.5 hours at reflux. The product (E) O-(3-trifluoromethoxybenzyl)hydroxylamine hydrochloride was obtained in a yield of 90%.

¹H NMR (DMSO-d6): δ9.79 (br s, 3H), 7.54 (dd, J=8.6, 7.9H, 1H), 7.43-7.36 (m, 3H), 4.90 (s, 2H).

### Example 8c

### Preparation of 6-(3-trifluoromethoxybenzyloxy)imino-2-methoxyestrone-17-oxime (CP-DM-4-67)

The same procedure was used as outlined in Example 4c above with the following variations.

As reagent F, (2-methoxy-6-oxo-estrone-17-oxime), was used in an amount of 14.1 mg (0.043 mmol), reagent E from Example 8b was used in an amount of 13.6 mg (0.056 mmol), resin G was used in an amount of 124 mg, and resin H was used in an amount of 35.0 mg. The reaction was conducted for 64 hours. Filtration was through a LC-CN solid phase extraction cartridge. The product, 2-methoxy-6-(3-trifluoromethoxybenzyloxy)iminoestrone-17-oxime, was obtained in a yield of 34%.

¹H NMR (CDCl₃): δ7.52 (s, 1H), 7.40-7.26 (m, 3H), 7.14 (d, 7.9 Hz, 1H), 6.78 (s, 1H), 5.50 (br s, 1H), 5.20 (s, 2H), 3.91 (s, 3H), 3.20 (dd, J=18.0, 4.5 Hz, 1H), 2.67-1.24 (m, 12H), 0.94 (s, 3H); ¹³C NMR (CDCl₃) δ170.93, 153.79, 149.25, 147.91, 143.91, 140.79, 135.01, 129.65, 123.51, 120.46 (q, J=257.1 Hz), 120.34, 119.97, 109.88, 106.74, 75.14, 55.83, 53.17, 43.94, 41.83, 36.48, 33.57, 29.42, 25.58, 25.02, 22.85, 17.00; ESI-MS (20v) m/z 519 (100) [M+H]⁺; HRESI-MS: [M+H]⁺ 519.2102 (519.2107 calc.)

### Example 9

### Preparation of 6-(3-trifluoromethoxybenzyloxy)imino-2-ethoxyestradiol (CP-DM-4-63)

The same procedure was used as outlined in Example 4c above with the following variations.

As reagent F, 2-methoxy-6-oxo-estradiol was used in an amount of 11.0 mg (0.035 mmol), reagent E from Example 8b was used in an amount of 11.0 mg (0.045 mmol), resin G was used in an amount of 141.3 mg, and resin H was used in an amount of 35 mg. The reaction was conducted for 64 hours. Filtration was through a LC-CN solid phase extraction cartridge. The product, 2-methoxy-6-(3-trifluoromethoxybenzyloxy)iminoestradiol was obtained in a yield of 25%.

¹H NMR (CDCl₃): δ7.49 (s, 1H), 7.37-7.23 (m, 3H), 7.12-7.11 (m, 1H), 6.76 (s, 1H), 5.43 (br s, 1H), 5.17 (s, 2H), 3.89 (s, 3H), 3.72 (t, J=8.5 Hz, 1H), 3.12 (dd, J=18.0, 4.4 Hz, 1H, 2.32-1.17 (m, 12H), 0.75 (s, 3H); ESI-MS (60v) m/z 506 (100) [M+H]⁺; HRESI-MS: [M+H]⁺ 506.2155 (506.2154 calc.)

### Example 10

### Preparation of 6-(4-trifluoromethoxybenzyloxy)imino-2-methoxyestrone-17-oxime

### CP-DM-4-68

This was prepared via 2 intermediates, in stages corresponding to those set out in Example 4.

### Example 10a

### Preparation of N-(4-trifluoromethoxybenzyloxy)phthalimide

The same procedure was used as outlined in Example 4a above with the following variations.

As reagent B, 4-trifluormethoxybenzyl bromide was used in 490.9 μL (3.07 mmol), reagent A was used in an amount of 455.0 mg (2.79 mmol), and 1.20 mL (7.01 mmol) of reagent C was used. The reaction was conducted for 15 hours at reflux. Extraction was with 1×15 mL ethyl acetate and 2×25 mL dichloromethane. The product D, N-(4-trifluoromethoxybenzyloxy)phthalimide, was obtained in a yield of 91%.

[δ¹H NMR (CDCl₃): δ7.82-7.70 (m, 4H), 7.57 (d, J=8.6 Hz, 2H), 7.21 (d, J=8.2 Hz, 2H), 5.18 (s, 2H).

### Example 10b

### Preparation of O-(4-trifluoromethoxybenzyl)hydroxylamine hydrochloride

The same procedure was used as outlined in Example 4b above with the following variations.

As reagent D, N-(4-trifluoromethoxybenzyloxy)phthalimide was used in 223.3 mg (0.695 mmol), glacial acetic acid was used in a 10 mL quantity and hydrochloric acid 37% was used in a 2.00 mL (63.6 mmol) quantity. The reaction was conducted for 4.5 hours at reflux. The product E, O-(4-trifluoromethoxybenzyl)hydroxylamine hydrochloride, was obtained in a yield of 93%.

¹H NMR (DMSO-d6): δ10.78 (br s, 3H), 7.55 (d, J=8.5 Hz, 2H), 7.42 (d, J=8.4 Hz, 2H), 5.01 (s, 2H).

### Example 10c

### Preparation of 6-(4-trifluoromethoxybenzyloxy)imino-2-methoxyestrone-17-oxime (CP-DM-4-68)

The same procedure was used as outlined in Example 4c above with the following variations.

As reagent F, 2-methoxy-6-oxo-estrone-17-oxime was used in an amount of 14.2 mg (0.043 mmol), reagent E from Example 10b was used in an amount of 13.7 mg (0.056 mmol), resin G was used in an amount of 133.4 mg, and resin H was used in an amount of 35.0 mg. The reaction was conducted for 64 hours. Filtration was through a LC-CN solid phase extraction cartridge. The product, 2-methoxy-6-(4-trifluoromethoxybenzyloxy)iminoestrone-17-oxime was obtained in a yield of 85%.

¹H NMR (CDCl₃): δ7.94 (br s, 1H), 7.53 (s, 1H), 7.43 (d, J=8.4 Hz, 2H), 7.20 (d, J=8.2 Hz, 2H), 6.77 (s, 1H), 5.57 (br s, 1H), 5.18 (s, 2H), 3.91 (s, 3H), 3.18 (dd, J=18.0, 4.5 Hz, 1H), 2.63-1.23 (m, 12H), 0.93 (s, 3H); ¹³C NMR (CDCl₃) δ170.71, 153.57, 148.67, 147.89, 143.91, 137.05, 134.95, 129.58 123.55, 120.80, 120.44 (q, J=257.2 Hz), 109.87, 106.75, 75.17, 55.83, 53.17, 43.95, 41.79, 36.45, 33.53, 29.40, 25.52, 25.05, 22.89, 16.98; ESI-MS (20v) m/z 519 (100) [M+H]⁺; HRESI-MS: [M+H]⁺ 519.2101 (519.2107 calc.)

### Example 11

### Preparation of 6-(4-trifluoromethoxybenzyloxy)imino-2-methoxyestradiol (CP-DM-4-64)

The same procedure was used as outlined in Example 4c above with the following variations.

As reagent F, 2-methoxy-6-oxo-estradiol was used in an amount of 11.4 mg (0.036 mmol), reagent E from Example 10b was used in an amount of 11.4 mg (0.047 mmol), resin G was used in an amount of 133.1 mg, and resin H was used in an amount of 35 mg. The reaction was conducted for 64 hours. Filtration was through a LC-CN solid phase extraction cartridge. The product, 2-methoxy-6-(4-trifluoromethoxybenzyloxy)iminoestradiol was obtained in a yield of 29%.

¹H NMR (CDCl₃): δ7.52 (s, 1H), 7.43 (d, J=8.7 Hz, 2H), 7.19 (d, J=7.9 Hz, 2H), 6.78 (s, 1H), 5.47 (br s, 1H), 5.17 (s, 2H), 3.91 (s, 3H), 3.74 (t, J=8.5 Hz, 1H), 3.12 (dd, J=18.1, 4.5 Hz, 1H), 2.34-1.17 (m, 12H), 0.76 (s, 3H); ESI-MS (20v) m/z 506 (100) [M+H]⁺; HRESI-MS: [M+H]⁺ 506.2149 (506.2154 calc.)

### Example 12

### Preparation of 6-(4-trifluoromethylthiobenzyloxy)imino-2-methoxyestrone-17-oxime (CP-DM-4-69)

This was prepared via 2 intermediates, in stages corresponding to those set out in Example 4.

### Example 12a

### Preparation of N-(4-trifluoromethylthiobenzyloxy)phthalimide

The same procedure was used as outlined in Example 4a above with the following variations.

As reagent B, 4-trifluoromethylthiobenzyl bromide was used in 747.8 mg (2.77 mmol), reagent A was used in an amount of 475.5 mg (2.91 mmol), and 1.20 mL (7.01 mmol) of reagent C was used. The reaction was conducted for 15 hours at reflux. Extraction was with 1×15 mL ethyl acetate and 2×25 mL dichloromethane. The product D, N-(4-trifluoromethylthiobenzyloxy)phthalimide, was obtained in a yield of 95%.

¹H NMR (CDCl₃): δ7.86-7.73 (m, 4H), 7.68 (d, J=8.2 Hz, 2H), 7.61 (d, J=8.2 Hz, 2H), 5.25 (s, 2H).

### Example 12b

### Preparation of O-(4-trifluoromethylthiobenzyl)hydroxylamine hydrochloride

The same procedure was used as outlined in Example 4b above with the following variations.

As reagent D, N-(4-trifluoromethylthiobenzyloxy)phthalimide was used in 203.1 mg (0.602 mmol), glacial acetic acid was used in a 10 mL quantity and hydrochloric acid 37% was used in a 2.00 mL (63.6 mmol) quantity. The reaction was conducted for 4.5 hours at reflux. The product E, O-(4-trifluoromethylthiobenzyl)hydroxylamine hydrochloride, was obtained in a yield of 84%.

¹H NMR (DMSO-d6): δ10.36 (br s, 3H), 7.76 (d, J=8.1 Hz, 2H), 7.54 (d, J=8.3 Hz, 2H), 4.99 (s, 2H)

### Example 12c

### Preparation of 6-(4-trifluoromethylthiobenzyloxy)imino-2-methoxyestrone-17-oxime (CP-DM-4-69)

The same procedure was used as outlined in Example 4c above with the following variations.

As reagent F, 2-methoxy-6-oxo-estrone-17-oxime was used in an amount of 15.5 mg (0.047 mmol), reagent E from Example 12b was used in an amount of 15.9 mg (0.061 mmol), resin G was used in an amount of 116.9 mg, and resin H was used in an amount of 35.0 mg. The reaction was conducted for 64 hours. Filtration was through a LC-CN solid phase extraction cartridge. The product, 2-methoxy-6-(4-trifluoromethylthiobenzyloxy)iminoestrone-17-oxime, was obtained in a yield of 68%.

¹H NMR (CDCl₃): δ7.90 (br s, 1H) 7.64 (d, J=8.1 Hz, 2H) 7.52 (s, 1H), 7.45 (d, J=8.1 Hz, 2H), 6.78 (s, 1H), 5.55 (br s, 1H), 5.22 (s, 2H), 3.91 (s, 3H), 3.21 (dd, J=17.9, 4.6 Hz, 1H), 2.64-1.39 (m, 12H), 0.94 (s, 3H); ¹³C NMR (CDCl₃) δ170.70, 153.76, 147.94, 143.93, 141.63, 136.30, 135.00, 129.57 (q, J=308 Hz), 128.84, 123.46, 109.90, 106.77, 75.15, 53.17, 43.97, 41.80, 36.45, 33.54, 29.42, 25.52, 25.08, 22.90, 16.99; ESI-MS (20v) m/z 535 (100) [M+H]⁺; HRESI-MS: [M+H]⁺ 535.1873 (535.1878 calc.)

### Example 13

### Preparation of 6-(4-trifluoromethylthiobenzyloxy)imino-2-methoxyestradiol (CP-DM-4-65)

The same procedure was used as outlined in Example 4c above with the following variations.

As reagent F, 2-methoxy-6-oxo-estradiol was used in an amount of 11.2 mg (0.035 mmol), reagent E from Example 12b was used in an amount of 11.9 mg (0.046 mmol), resin G was used in an amount of 133.8 mg, and resin H was used in an amount of 35 mg. The reaction was conducted for 64 hours. Filtration was through a LC-CN solid phase extraction cartridge. The product, 2-methoxy-6-(4-trifluoromethylthiobenzyloxy)iminoestradiol, was obtained in a yield of 87%.

¹H NMR (CDCl₃): δ7.63 (d, J=8.2 Hz, 2H) 7.51 (s, 1H), 7.44 (d, J=8.3 Hz, 2H), 6.78 (s, 1H), 5.49 (br s, 1H), 5.21 (s, 2H), 3.90 (s, 3H), 3.16 (dd, J=18.1, 4.6 Hz, 1H), 2.34-1.18 (m, 12H), 0.77 (s, 3H); ESI-MS (20v) m/z 522 (100) [M+H]⁺; HRESI-MS: [M+H]⁺ 522.1922 (522.1926 calc.)

### Example 14

### Preparation of 6-(4-pyridylmethyloxy)imino-2-methoxyestrone-17-oxime (CP-DM-4-15)

This was prepared via 2 intermediates, in stages corresponding to those set out in Example 4.

### Example 14a

### Preparation of N-(4-pyridylmethyloxy)phthalimide

The same procedure was used as outlined in Example 4a above with the following variations.

As reagent B, (4-bromomethyl)pyridine hydrobromide was used in 2.048 g (8.098 mmol), reagent A was used in an amount of 1.201 g (7.362 mmol), and 3.665 mL (22.08 mmol) of reagent C was used. The reaction was conducted for 136 hours at ambient temperature in 50.0 mL of tetrahydrofuran. Extraction was with 3×50 mL ethyl acetate. Combined extracts were washed with water (50 mL), 50% saturated brine (100 mL) and brine (50 mL). The product D, N-(4-pyridylmethyloxy)phthalimide, was recrystallised from toluene and obtained in a yield of 34%.

¹H NMR (CDCl₃): δ8.66 (d, J=5.4 Hz, 2H), 7.85-7.74 (m, 4H), 7.48 (d, J=5.6 Hz, 2H), 5.24 (s, 2H).

### Example 14b

### Preparation of O-(4-pyridylmethyl)hydroxylamine dihydrochloride

The same procedure was used as outlined in Example 4b above with the following variations.

As reagent D, N-(4-pyridylmethyloxy)phthalimide was used in 103.3 mg (0.602 mmol), hydrochloric acid 37% was used in a 2.00 mL (63.6 mmol) quantity. The reaction was conducted for 2.5 hours at reflux. The product O-(4-pyridylmethyl)hydroxylamine dihydrochloride (E) was obtained in a yield of 50%.

¹H NMR (CD₃OD): δ8.90 (d, J=6.1 Hz, 2H), 8.03 (d, J=6.1 Hz, 2H), 5.26 (s, 2H).

### Example 14c

### Preparation of 6-(4-pyridylmethyloxy)imino-2-methoxyestrone-17-oxime (CP-DM-4-15)

The same procedure was used as outlined in Example 4c above with the following variations.

As reagent F, 2-methoxy-6-oxo-estrone-17-oxime was used in an amount of 16.5 mg (0.050 mmol), reagent E from Example 14b was used in an amount of 12.8 mg (0.065 mmol), resin G was used in an amount of 172.4 mg, and resin H was used in an amount of 37.1 mg. The reaction was conducted for 90 hours. Purification was performed by flash chromatography on silica gel [ethyl acetate-petroleum spirit (40-60° C.) (3:1)]. The product, 2-methoxy-6-(4-pyridylmethyloxy)iminoestrone-17-oxime, was obtained in a yield of 46%.

¹H NMR (DMSO-d6): δ10.13 (br s, 1H), 8.54 (d, J=4.7 Hz, 2H), 7.35 (d, J=4.8 Hz, 2H), 7.22 (s, 1H), 6.81 (s, 1H), 5.19 (s, 2H), 3.77 (s, 3H), 3.14 (dd, J=17.6, 4.5 Hz, 1H), 2.56-1.20 (m, 12H), 0.83 (s, 3H); ¹³C NMR (CDCl₃+DMSO-d6) δ154.85, 149.67, 149.29, 149.12, 144.98, 134.98, 122.38, 110.53, 110.49, 108.64, 80.29, 73.56, 55.78, 50.17, 43.02, 41.75, 37.51, 36.45, 30.26, 29.52, 25.68, 23.08, 11.48; ESI-MS (20v) m/z 436 (100) [M+H]⁺; HRESI-MS: [M+H]⁺ 436.2225 (436.2236 calc.)

### Example 15

### Preparation of 6-(4-pyridylmethyloxy)imino-2-methoxyestradiol (CP-DM-4-16)

The same procedure was used as outlined in Example 4c above with the following variations.

As reagent F, 2-methoxy-6-oxo-estradiol was used in an amount of 13.7 mg (0.043 mmol), reagent E from Example 14b was used in an amount of 11.1 mg (0.056 mmol), resin G was used in an amount of 140.2 mg, and resin H was used in an amount of 32.5 mg. The reaction was conducted for 90 hours. Purification was performed by flash chromatography on silica gel [ethyl acetate-petroleum spirit (40-60° C.) (3:1)]. The product, 2-methoxy-6-(4-pyridylmethyloxy)iminoestradiol, was obtained in a yield of 52%.

¹H NMR (CDCl₃): δ8.58 (d, J=4.9 Hz, 1H), 7.47 (s, 2H), 7.33 (d, J=4.9 Hz, 2H), 6.78 (s, 1H), 5.21 (s, 2H), 3.90 (s, 3H), 3.75 (t, J=8.4 Hz), 3.16 (dd, J=18.0, 4.5 Hz, 1H), 2.37-1.20 (m, 12H), 0.78 (s, 3H); ESI-MS (20v) m/z 423 (100) [M+H]⁺; HRESI-MS: [M+H]⁺ 423.2271 (423.2283 calc.)

### Example 16

### Preparation of 6-(4-cyanobenzyloxy)imino-2-methoxyestrone-17-oxime (CP-DM-4-38)

This was prepared via 2 intermediates, in stages corresponding to those set out in Example 4.

### Example 16a

### Preparation of N-(4-cyanobenzyloxy)phthalimide

The same procedure was used as outlined in Example 4a above with the following variations.

As reagent B, 4-cyanobenzyl bromide was used in 773.4 mg (3.95 mmol), reagent A was used in an amount of 585.0 mg (3.59 mmol), and 1.35 mL (7.89 mmol) of reagent C was used. The reaction was conducted for 20 hours at reflux. Extraction was with 3×50 mL dichloromethane. Combined extracts were washed with water (50 mL), 50 mL brine. The product D, N-(4-cyanobenzyloxy)phthalimide, was obtained in a yield of 73%.

¹H NMR (DMSO-d6): δ7.89 (d, J=8.2 Hz, 2H), 7.85 (m, 4H), 7.73 (d, J=8.3 Hz, 2H), 5.27 (s, 2H).

### Example 16b

### Preparation of O-(4-cyanobenzyl)hydroxylamine hydrochloride

The same procedure was used as outlined in Example 4b above with the following variations.

As reagent D, N-(4-cyanobenzyloxy)phthalimide was used in 143.1 mg (0.514 mmol). The reaction was conducted for 3 hours at reflux. The product O-(4-cyanobenzyl)hydroxylamine hydrochloride (E) was obtained in a yield of 70%.

¹H NMR (DMSO-d6): δ7.89 (d, J=8.1 Hz, 2H), 7.59 (d, J=7.9 Hz, 2H), 5.02 (s, 2H).

### Example 16c

### Preparation of 6-(4-cyanobenzyloxy)imino-2-methoxyestrone-17-oxime (CP-DM-4-38)

The same procedure was used as outlined in Example 4c above with the following variations.

As reagent F, 2-methoxy-6-oxo-estrone-17-oxime was used in an amount of 14.0 mg (0.043 mmol), reagent E from Example 16b was used in an amount of 10.2 mg (0.055 mmol), resin G was used in an amount of 200 mg, and resin H was used in an amount of 48.0 mg. The reaction was conducted for 64 hours. The product, 2-methoxy-6-(4-cyanobenzyloxy)iminoestrone-17-oxime, was obtained in a yield of 74%.

¹H NMR (CDCl₃): δ7.64 (d, J=8.2 Hz, 2H), 7.49 (d, J=8.4 Hz, 2H), 7.48 (s, 1H), 6.77 (s, 1H), 5.55 (br s, 1H), 5.22 (s, 2H), 3.91 (s, 3H), 3.20 (dd, J=18.0, 4.4 Hz, 1H), 2.60-1.25 (m, 12H), 0.94 (s, 3H); ESI-MS (20v) m/z 460 (100) [M+H]⁺; HRESI-MS: [M+H]⁺ 460.2235 (460.2236 calc.)

### Example 17

### Preparation of 6-(4-cyanobenzyloxy)imino-2-methoxyestradiol (CP-DM-4-37)

The same procedure was used as outlined in Example 4c above with the following variations.

As reagent F, 2-methoxy-6-oxo-estradiol was used in an amount of 11.7 mg (0.037 mmol), reagent E from Example 16b was used in an amount of 8.9 mg (0.046 mmol), resin G was used in an amount of 200 mg, and resin H was used in an amount of 49 mg. The reaction was conducted for 64 hours. The product, 2-methoxy-6-(4-cyanobenzyloxy)iminoestradiol, was obtained in a yield of 77%.

¹H NMR (CDCl₃): δ7.64 (d, J=8.0 Hz, 2H), 7.48 (d, J=8.6 Hz, 2H), 7.47 (s, 1H), 6.78 (s, 1H), 5.49 (br s, 1H), 5.22 (s, 2H), 3.90 (s, 3H), 3.73 (t, J=9.0 Hz, 1H), 3.13 (dd, J=18.0, 4.5 Hz, 1H), 2.18-1.22 (m, 12H), 0.77 (s, 3H); ¹³C NMR (CDCl₃) δ154.41, 147.95, 144.14, 143.82, 135.46, 132.15, 128.24, 123.36, 118.92, 111.26, 109.78, 106.80, 81.58, 74.87, 55.82, 50.43, 43.00, 41.77, 37.20, 36.16, 30.52, 29.52, 25.67, 23.07, 10.94; ESI-MS (60v) m/z 447 (100) [M+H]⁺; HRESI-MS: [M+H]⁺ 447.2275 (447.2284 calc.)

### Example 18

### Preparation of 6-(3-cyanobenzyloxy)imino-2-methoxyestrone-17-oxime (CP-DM-4-51)

This was prepared via 2 intermediates, in stages corresponding to those set out in Example 4.

### Example 18a

### Preparation of N-(3-cyanobenzyloxy)phthalimide

The same procedure was used as outlined in Example 4a above with the following variations.

As reagent B, 3-cyanobenzyl bromide was used in 773.4 mg (3.95 mmol), reagent A was used in an amount of 585.0 mg (3.59 mmol), and 1.35 mL (7.89 mmol) of reagent C was used. The reaction was conducted for 20 hours at reflux. Extraction was with 3×50 mL dichloromethane. Combined extracts were washed with water (50 mL), 50 mL brine. The product D, N-(3-cyanobenzyloxy)phthalimide, was obtained in a yield of 73%.

¹H NMR (CDCl₃): δ7.85-7.75 (m, 6H), 7.68 (d, J=7.9 Hz, 1H), 7.53 (dd, J=8.1, 7.8 Hz, 1H), 5.23 (s, 2H).

### Example 18b

### Preparation of O-(3-cyanobenzyl)hydroxylamine hydrochloride

The same procedure was used as outlined in Example 4b above with the following variations.

As reagent D, N-(3-cyanobenzyloxy)phthalimide was used in 143.1 mg (0.514 mmol). The reaction was conducted for 3 hours at reflux. The product O-(3-cyanobenzyl)hydroxylamine hydrochloride (E) was obtained in a yield of 70%.

¹H NMR (DMSO-d6): δ10.75 (br s, 3H), 7.88-7.85 (m, 2H), 7.75 (d, J=7.9 Hz, 1H), 7.64 (dd, J=7.9, 7.8 Hz, 1H), 5.05 (s, 2H).

### Example 18c

### CP-DM-4-51

### Preparation of 6-(3-cyanobenzyloxy)imino-2-methoxyestrone-17-oxime

The same procedure was used as outlined in Example 4c above with the following variations.

As reagent F, 2-methoxy-6-oxo-estrone-17-oxime was used in an amount of 14.0 mg (0.043 mmol), reagent E from Example 18b was used in an amount of 10.2 mg (0.055 mmol), resin G was used in an amount of 200 mg, and resin H was used in an amount of 48.0 mg. The reaction was conducted for 64 hours. Purification was performed by flash chromatography on silica gel [ethyl acetate-petroleum spirit (40-60° C.) (2:3)]. The product, 2-methoxy-6-(3-cyanobenzyloxy)iminoestrone-17-oxime, was obtained in a yield of 74%.

¹H NMR (CDCl₃): δ7.78 (br, s 1H), 7.68 (s, 1H), 7.62 (d, J=7.8 Hz, 1H), 7.59 (d, J=7.7 Hz, 1H), 7.48-7.44 (m, 2H), 6.78 (s, 1H), 5.55 (br s, 1H), 5.20 (s, 2H), 3.91 (s, 3H), 3.22 (dd, J=17.9, 4.4 Hz, 1H), 2.65-1.25 (m, 12H), 0.94 (s, 3H); ¹³C NMR (CDCl₃) δ171.19, 154.00, 148.02, 143.94, 140.11, 135.02, 132.26, 131.38, 131.27, 129.10, 123.32, 118.89, 112.44, 109.85, 106.77, 74.73, 55.85, 53.19, 44.07, 41.77, 36.45, 33.51, 29.40, 25.54, 25.27, 22.88, 16.97; ESI-MS (20v) m/z 460 (100) [M+H]⁺; HRESI-MS: [M+H]⁺ 460.2231 (460.2236 calc.)

### Example 19

### Preparation of 6-(3-cyanobenzyloxy)imino-2-methoxyestradiol (CP-DM-4-52)

The same procedure was used as outlined in Example 4c above with the following variations.

As reagent F, 2-methoxy-6-oxo-estradiol, was used in an amount of 11.7 mg (0.037 mmol), reagent E from Example 18b as used in an amount of 8.9 mg (0.046 mmol), resin G was used in an amount of 200 mg, and resin H was used in an amount of 49 mg. The reaction was conducted for 64 hours. The product, 2-methoxy-6-(3-cyanobenzyloxy)iminoestradiol, was obtained in a yield of 77%.

¹H NMR (CDCl₃): δ7.67 (s, 1H), 7.61 (d, J=7.7 Hz, 1H), 7.57 (d, J=7.7 Hz, 1H), 7.53-7.43 (m, 2H), 6.78 (s, 1H), 5.54 (br s, 1H), 5.19 (s, 2H), 3.90 (s, 3H), 3.74 (t, J=8.5 Hz, 1H) 3.12 (dd, J=18.1, 4.5 Hz, 1H), 2.34-1.22 (m, 12H), 0.77 (s, 3H); ¹³C NMR (CDCl₃) δ154.43, 147.95, 143.82, 140.23, 135.48, 132.20, 131.31, 131.22, 129.07, 123.39, 118.89, 112.41, 109.80, 106.83, 81.59, 74.64, 55.82, 50.43, 43.00, 41.76, 37.20, 36.17, 30.53, 29.53, 25.67, 23.07, 10.93; ESI-MS (20v) m/z 447 (100) [M+H]⁺; HRESI-MS: [M+H]⁺ 447.2283 (447.2284 calc.)

### Example 20

### Preparation of 6-(4-methylbenzyloxy)imino-2-methoxyestrone-17-oxime (CP-DM-4-76)

This is prepared via two intermediates, as follows.

### Example 20a

### Preparation of N-(4-methylbenzyloxy)phthalimide

The same procedure was used as outlined in Example 4 above with the following variations.

As reagent B, 4-methylbenzyl bromide was used in 590.3 μL (2.90 mmol), reagent A was used in an amount of 590.3 mg (3.19 mmol), and 1.20 mL (7.01 mmol) of reagent C was used. The reaction was conducted for 15 hours at reflux. Extraction was with 1×15 mL ethyl acetate and 2×25 mL dichloromethane. The product D, N-(4-methylbenzyloxy)phthalimide, was obtained in a yield of 90%.

¹H NMR (CDCl₃): δ7.82-7.72 (m, 4H), 7.42 (d, J=8.1 Hz, 1H), 7.18 (d, J=7.9 Hz, 2H), 5.17 (s, 2H), 2.35 (s, 3H).

### Example 20b

### Preparation of O-(4-methylbenzyl)hydroxylamine hydrochloride

The same procedure was used as outlined in Example 4b above with the following variations.

As reagent D, N-(4-methylbenzyloxy)phthalimide was used in 214.8 mg (0.855 mmol), glacial acetic acid was used in a 10 mL quantity and hydrochloric acid 37% was used in a 2.00 mL (63.6 mmol) quantity. The reaction was conducted for 4.5 hours at reflux. The product O-(4-methylbenzyl)hydroxylamine hydrochloride (E) was obtained in a yield of 21%.

¹H NMR (DMSO-d6): δ9.77 (br s, 3H), 7.26 (d, J=8.0 Hz, 2H), 7.20 (d, J=7.9 Hz, 2H), 4.81 (s, 2H), 2.30 (s, 3H).

### Example 20c

### Preparation of 6-(4-methylbenzyloxy)imino-2-methoxyestrone-17-oxime (CP-DM-4-76)

The same procedure was used as outlined in Example 4c above with the following variations.

As reagent F, 2-methoxy-6-oxo-estrone-17-oxime was used in an amount of 8.8 mg (0.027 mmol), reagent E from Example 20b was used in an amount of 6.0 mg (0.035 mmol), resin G was used in an amount of 107.1 mg, and resin H was used in an amount of 40.3 mg. The reaction was conducted for 90 hours. Filtration was through a LC-CN solid phase extraction cartridge. The product, 2-methoxy-6-(4-methylbenzyloxy)iminoestrone-17-oxime, was obtained in a yield of 43%.

¹H NMR (CDCl₃): δ7.56 (s, 1H), 7.32 (d, J=7.8 Hz, 2H), 7.17 (d, J=7.9 Hz, 2H), 6.77 (s, 1H), 5.50 (br s, 1H), 5.15 (s, 2H), 3.91 (s, 3H), 3.22 (dd, J=18.1, 4.4 Hz, 1H), 2.63-1.24 (m, 12H), 2.35 (s, 3H), 0.92 (s, 3H); ESI-MS (20v) m/z 449 (100) [M+H]⁺; HRESI-MS: [M+H]⁺ 449.2435 (449.2440 calc.)

### Example 21

### Preparation of 6-(4-methylbenzyloxy)imino-2-methoxyestradiol (CP-DM-4-78)

The same procedure was used as outlined in Example 4c above with the following variations.

As reagent F, 2-methoxy-6-oxo-estradiol was used in an amount of 6.2 mg (0.020 mmol), reagent E from Example 20b was used in an amount of 4.4 mg (0.025 mmol), resin G was used in an amount of 120.4 mg, and resin H was used in an amount of 22.3 mg. The reaction was conducted for 90 hours. Filtration was through a LC-CN solid phase extraction cartridge. The product, 2-methoxy-6-(4-methylbenzyloxy)iminoestradiol, was obtained in a yield of 25%.

¹H NMR (CDCl₃): δ7.54 (s, 1H), 7.31 (d, J=7.7 Hz, 2H), 7.16 (d, J=7.7 Hz, 2H), 6.76 (s, 1H), 5.45 (br s, 1H), 5.14 (s, 2H), 3.90 (s, 3H), 3.10 (dd, J=18.1, 4.6 Hz, 1H), 2.34 (s, 3H), 2.33-1.17 (m, 12H), 0.75 (s, 3H); ESI-MS (20v) m/z 436 (100) [M+H]⁺.

### Example 22

### Preparation of 6-(4-isopropylbenzyloxy)imino-2-methoxyestrone-17-oxime (CP-DM-4-77)

This is prepared via two intermediates, as follows.

### Example 22a

### Preparation of N-(4-isopropylbenzyloxy)phthalimide

The same procedure was used as outlined in Example 4 above with the following variations.

As reagent B, 4-isopropylbenzyl bromide was used in 563.0 μL (2.99 mmol), reagent A was used in an amount of 488.5 mg (3.29 mmol), and 1.20 mL (7.01 mmol) of reagent C was used. The reaction was conducted for 15 hours at reflux. Extraction was with 1×15 mL ethyl acetate and 2×25 mL dichloromethane. The product D, N-(4-isopropylbenzyloxy)phthalimide, was obtained in a yield of 98%.

¹H NMR (CDCl₃): δ7.86-7.71 (m, 4H), 7.47 (d, J=8.0 Hz, 2H), 7.24 (d, J=8.1 Hz, 2H), 5.18 (s, 2H), 2.91 (septet, J=6.9 Hz, 1H), 1.24 (s, 3H), 1.23 (s, 3H).

### Example 22b

### Preparation of O-(4-isopropylbenzyl)hydroxylamine hydrochloride

The same procedure was used as outlined in Example 4b above with the following variations.

As reagent D, N-(4-isopropylbenzyloxy)phthalimide was used in 208.6 mg (0.747 mmol), glacial acetic acid was used in a 10 mL quantity and hydrochloric acid 37% was used in a 2.00 mL (63.6 mmol) quantity. The reaction was conducted for 4.5 hours at reflux. The product O-(4-isopropylbenzyl)hydroxylamine hydrochloride (E) was obtained in a yield of 24%.

¹H NMR (DMSO-d6): δ7.33-7.20 (m, 4H), 4.80 (s, 2H), 2.89 (septet, J=7.0 Hz, 1H), 1.20 (s, 3H), 1.18 (s, 3H).

### Example 22c

### Preparation of 6-(4-isopropylbenzyloxy)imino-2-methoxyestrone-17-oxime (CP-DM-4-77)

The same procedure was used as outlined in Example 4c above with the following variations.

As reagent F, 2-methoxy-6-oxo-estrone-17-oxime was used in an amount of 6.3 mg (0.019 mmol), reagent E from Example 22b was used in an amount of 4.1 mg (0.025 mmol), resin G was used in an amount of 106.4 mg, and resin H was used in an amount of 35.1 mg. The reaction was conducted for 90 hours. Filtration was through a LC-CN solid phase extraction cartridge. The product, 2-methoxy-6-(4-isopropylbenzyloxy)iminoestrone-17-oxime, was obtained in a yield of 21%.

¹H NMR (CDCl₃): δ7.57 (s, 1H), 7.35 (d, J=8.1 Hz, 2H), 7.22 (d, J=8.1 Hz, 2H), 6.77 (s, 1H), 5.47 (br s, 1H), 5.16 (s, 2H), 3.91 (s, 3H), 3.19 (dd, J=17.9, 4.6 Hz, 1H), 2.91 (septet, J=7.0 Hz, 1H) 2.63-1.20 (m, 12H), 1.44 (s, 3H), 1.43 (s, 3H), 0.92 (s, 3H); ESI-MS (20v) m/z 477 (100) [M+H]⁺.

### Example 23

### Preparation of 6-(3-nitrobenzyloxy)imino-2-methoxyestrone-17-oxime (CP-DM-3-105)

This is prepared via two intermediates, as follows.

### Example 23a

### Preparation of N-(3-nitrobenzyloxy)phthalimide

To a solution of N-hydroxyphthalimide (reagent A) (1.059 g, 6.418 mmol) in 25 mL anhydrous tetrahydrofuran under a nitrogen atmosphere was added 3-nitrobenzyl bromide (reagent B) (1.543 mL, 7.141 mmol). N,N-diisopropylethylamine (reagent C) (2.155 mL, 12.98 mmol) was then added dropwise with stirring to effect an immediate colour change. The reaction was then held at ambient temperature for 19 hours. The reaction mixture was then diluted with 25 mL of tetrahydrofuran and 100 mL of water prior to extraction with 3×100 mL ethyl acetate. Combined organic fractions were washed with water (100 mL) and brine (100 mL) prior to drying over anhydrous sodium sulphate. Purification was performed by flash chromatography on silica gel [dichloromethane]. Evaporation in vacuo gave 877.6 mg of the product (D) (45%) as a white solid.

¹H NMR (CDCl₃): δ8.39 (dd, J=1.9, 1.9 Hz, 1H), 8.25 (dd, J=8.2, 2.3 Hz, 1H), 7.95 (d, J=7.7 Hz, 1H), 7.60 (dd, J=8.0, 7.9 Hz, 1H), 7.84-7.75 (m, 4H), 5.30 (s, 2H).

### Example 23b

### Preparation of O-(3-nitrobenzyl)hydroxylamine hydrochloride

N-(3-nitrobenzyloxy)phthalimide (D from Example 23b) (166.2 mg, 0.557 mmol) was added to 15 mL ethanol and an amount of dichloromethane (7.5 mL) added. Hydrazine hydrate (reagent I) (29.8 μL, 0.613 mmol) was added to the reaction with stirring under a nitrogen atmosphere. The reaction was heated to reflux and held at that temperature for 18 hours before cooling to ambient temperature. The reaction was evaporated in vacuo and suspended in 5 mL ethanol and the suspension stirred at ambient temperature for 2 hours, prior to the solids being removed by filtration. The residues were washed with a further 3 mL of ethanol. The filtrate and washings were combined and evaporated, and purification performed by flash chromatography on silica gel [ethyl acetate-petroleum spirit (40-60° C.) (2:3)]. Dissolution of the resulting oil in ethanol (2 mL) followed by addition of hydrogen chloride (1 M in diethyl ether) and diethyl ether 20 mL gave a white precipitate. Filtration of the solid that was further washed with diethyl ether gave the product (E) in a yield of 94%.

¹H NMR (CD₃OD): δ8.39-8.20 (m, 2H), 7.84 (d, J=7.6 Hz, 1H), 7.70 (dd, J=8.1, 7.9 Hz, 1H), 5.11 (s, 2H).

### Example 23c

### Preparation of 2-methoxy-6-(3-nitrobenzyloxy)imino-estrone-17-oxime (CP-DM-3-105)

The same procedure was used as outlined in Example 4c above with the following variations.

As reagent F, 2-methoxy-6-oxo-estrone-17-oxime was used in an amount of 14.7 mg (0.045 mmol), reagent E from Example 23b was used in an amount of 11.9 mg (0.058 mmol), resin G was used in an amount of 72.1 mg, and resin H was used in an amount of 33.5 mg. The reaction was conducted for 28 hours. Filtration was through a polyethylene frit. The product, 2-methoxy-6-(3-nitrobenzyloxy)iminoestrone-17-oxime, was obtained in a yield of 68%.

¹H NMR (CDCl₃): δ8.26 (s, 1H), 8.16 (d, J=8.1 Hz, 1H), 7.73 (d, J=7.6 Hz, 1H), 7.51 (dd, J=8.0, 7.9 Hz, 1H), 7.49 (s, 1H), 7.41 (br s, 1H), 6.78 (s, 1H), 5.52 (br s, 1H), 5.27 (s, 2H), 3.91 (s, 3H), 3.23 (dd, J=18.0, 4.4 Hz, 1H), 2.63-1.22 (m, 12H), 0.94 (s, 3H); ¹³C NMR (CDCl₃) δ170.81, 154.22, 148.31, 148.02, 143.91, 140.78, 135.12, 133.96, 129.25, 123.29, 122.80, 122.63, 109.85, 106.77, 74.64, 55.84, 53.15, 43.95, 41.83, 36.48, 33.55, 29.44, 25.55, 25.05, 22.88, 17.01; ESI-MS (20v) m/z 480 (100) [M+H]⁺; HRESI-MS: [M+H]⁺ 480.2135 (480.2135 calc.)

### Example 24

### Preparation of 2-methoxy-6-(3-nitrobenzyloxy)iminoestradiol (CP-DM-3-106)

The same procedure was used as outlined in Example 4c above with the following variations.

As reagent F, 2-methoxy-6-oxo-estradiol was used in an amount of 16.3 mg (0.052 mmol), reagent E from Example 23b was used in an amount of 26.3 mg (0.129 mmol), resin G was used in an amount of 60 mg, and resin H was used in an amount of 193 mg. The reaction was conducted for 60 hours. Filtration was through a polyethylene frit. The product, 2-methoxy-6-(3-nitrobenzyloxy)iminoestradiol, was obtained in a yield of 92%.

¹H NMR (CDCl₃): δ8.25 (d, J=1.6 Hz, 1H), 8.15 (dd, J=8.1 Hz, 2.1 Hz, 1H), 7.72 (d, J=7.7 Hz, 1H), 7.52 (dd, J=8.1, 7.9 Hz, 1H), 7.48 (s, 1H), 6.78 (s, 1H), 5.49 (br s, 1H), 5.26 (s, 2H), 3.90 (s, 3H), 3.75 (t, J=8.6 Hz, 1H), 3.15 (dd, J=18.0, 4.6 Hz, 1H), 2.36-1.20 (m, 12H), 0.77 (s, 3H); ¹³C NMR (CDCl₃) δ154.60, 148.30, 147.96, 143.80, 140.80, 135.52, 133.90, 129.23, 123.36, 122.74, 122.57, 109.80, 106.82, 81.60, 74.55, 55.81, 50.41, 43.00, 41.77, 37.21, 36.15, 30.53, 29.56, 25.67, 23.05, 10.93; ESI-MS (20v) m/z 467 (100) [M+H]⁺; HRESI-MS: [M+H]⁺ 467.2179 (467.2182 calc.)

### Example 25

### Preparation of 2-methoxy-6-(2-nitrobenzyloxy)iminoestrone-17-oxime (CP-DM-3-117)

This is prepared via two intermediates, as follows.

### Example 25a

### Preparation of N-(2-nitrobenzyloxy)phthalimide

The same procedure was used as outlined in Example 23a above with the following variations.

As reagent B, 2-nitrobenzyl bromide was used in 1.6306 g (7.548 mmol), reagent A from Example 23a was used in an amount of 1.1193 g (6.861 mmol), and 2.277 mL (13.72 mmol) of reagent C was used. The reaction was conducted for 41 hours at ambient temperature. A further quantity of reagent B [2-nitrobenzyl bromide] (313.5 mg, 1.45 mmol) was added to the reaction mixture and the reaction continued for a further 48 hours at ambient temperature. The reaction was diluted with water (50 mL). The product D, N-(2-nitrobenzyloxy)phthalimide, was obtained by recrystallization from chloroform in a yield of 51%.

¹H NMR (CDCl₃) δ8.14 (d, J=8.3 Hz, 1H), 8.01 (d, J=7.8 Hz), 7.90-7.68 (m, 5H), 7.54 (dd, J=8.3, 7.8 Hz, 1H), 5.65 (s, 2H).

### Example 25b

### Preparation of O-(2-nitrobenzyl)hydroxylamine hydrochloride

The same procedure was used as outlined in Example 23b above with the following variations.

As reagent D, N-(2-nitrobenzyloxy)phthalimide was used in 188.4 mg (0.632 mmol), reagent I was used in an amount of 33.8 μL (0.695 mmol). The reaction was conducted in ethanol 15 mL for 20 hours at reflux. A further quantity of reagent I was added (5 μL, 0.103 mmol) and the reaction continued for a further 24 hours at reflux. The product O-(2-nitrobenzyl)hydroxylamine hydrochloride (E) was obtained in a yield of 23%.

¹H NMR (CD₃OD) δ8.17 (d, J=8.3 Hz, 1H), 7.51-7.83 (m 3H), 5.44 (s, 2H).

### Example 25c

### Preparation of 2-methoxy-6-(2-nitrobenzyloxy)iminoestrone-17-oxime (CP-DM-3-117)

The same procedure was used as outlined in Example 4c above with the following variations.

As reagent F, 2-methoxy-6-oxo-estrone-17-oxime was used in an amount of 13.5 mg (0.041 mmol), reagent E from Example 25b was used in an amount of 10.5 mg (0.051 mmol), resin G was used in an amount of 80 mg, and resin H was used in an amount of 35 mg. The reaction was conducted for 66 hours. The product, 2-methoxy-6-(2-nitrobenzyloxy)iminoestrone-17-oxime, was obtained in a yield of 88%.

¹H NMR (CDCl₃): δ8.05 (d, J=8.1 Hz, 1H), 7.61 (m, 2H), 7.46-7.40 m, 2H), 6.77 (s, 1H), 5.59 (s, 2H), 3.91 (s, 3H), 3.25 (dd, J=18.1, 4.6 Hz, 1H), 2.71-1.24 (m, 12H), 1.00 (s, 3H); ESI-MS (20v) m/z 480 (100) [M+H]⁺; HRESI-MS: [M+H]⁺ 480.2136 (480.2135 calc.)

### Example 26

### Preparation of 2-methoxy 6-(2-nitrobenzyloxy)iminoestradiol (CP-DM-3-124)

The same procedure was used as outlined in Example 4c above with the following variations.

As reagent F, 2-methoxy-6-oxo-estradiol was used in an amount of 16.3 mg (0.052 mmol), reagent E from Example 25b was used in an amount of 15.8 mg (0.077 mmol), resin G was used in an amount of 98.3 mg, and resin H was used in an amount of 52 mg. The reaction was conducted for 48 hours. The product was purified by dissolution in methanol (8 mL) and filtration through a C18 cartridge (loading 300 mg). The product, 2-methoxy-6-(2-nitrobenzyloxy)iminoestradiol, was obtained in a yield of 99%.

¹H NMR (CDCl₃): δ8.06 (d, J=8.4 Hz, 1H), 7.63-7.57 (m, 2H), 7.46-7.38, m, 2H), 7.49 (s, 1H), 6.78 (s, 1H), 5.59 (s, 2H), 3.90 (s, 3H), 3.76 (t, J=8.6 Hz, 1H), 3.19 (dd, J=18.1, 4.5 Hz, 1H), 2.34-1.20 (m, 12H), 0.77 (s, 3H); ESI-MS (20v) m/z 467 (100) [M+H]⁺; HRESI-MS: [M+H]⁺ 467.2179 (467.2182 calc.)

### Example 27

### Preparation of 2-methoxy-6-(4-methoxybenzyloxy)iminoestrone-17-oxime (CP-DM-4-6)

This is prepared via two intermediates, as follows.

### Example 27a

### Preparation of N-(4-methoxybenzyloxy)phthalimide

The same procedure was used as outlined in Example 23a above with the following variations.

As reagent B, 4-methoxybenzyl chloride was used in 0.9777 g (7.204 mmol), reagent A from Example 23a was used in an amount of 1.0684 g (6.549 mmol), and 2.173 mL (13.10 mmol) of reagent C from Example 23a was used. The reaction was conducted for 41 hours at ambient temperature. A further quantity of reagent B [4-methoxybenzyl chloride] (250.0 μL, 1.835 mmol) was added to the reaction mixture and the reaction continued for a further 40 hours at ambient temperature. The reaction was diluted with water (50 mL). The product D, N-(4-methoxybenzyloxy)phthalimide, was in a crude yield of 72%.

¹H NMR (CDCl₃) inter alia 87.83-7.77 (m, 2H), 7.76-7.70 (m, 2H), 7.45 (d, J=8.4 Hz, 2H), 7.29 (d, J=8.4 Hz, 2H), 5.15 (s, 2H), 3.81 (s, 3H).

### Example 27b

### Preparation of O-(4-methoxybenzyl)hydroxylamine hydrochloride

The same procedure was used as outlined in Example 23b above with the following variations.

As crude reagent D, N-(4-methoxybenzyloxy)phthalimide was used in 291.9 mg (1.030 mmol), and reagent X was used in an amount of 101.3 μL (2.084 mmol). The reaction was conducted in ethanol 6 mL and 3 ml dichloromethane for 21 hours at reflux. The product O-(4-methoxybenzyl)hydroxylamine hydrochloride (E) was obtained in a yield of 23%.

¹H NMR (CD₃OD) δ7.36 (d, J=8.7 Hz, 2H), 6.97 (d, J=8.7 Hz, 2H), 4.92 (s, 2H), 3.81 (s, 3H).

### Example 27c

### Preparation of 2-methoxy-6-(4-methoxybenzyloxy)iminoestrone-17-oxime (CP-DM-4-6)

The same procedure was used as outlined in Example 4c above with the following variations.

As reagent F, (2-methoxy-6-oxo-estrone-17-oxime), was used in an amount of 19.7 mg (0.060 mmol), reagent E from Example 27b was used in an amount of 11.5 mg (0.075 mmol), resin G was used in an amount of 100.4 mg, and resin H was used in an amount of 117.6 mg. The reaction was conducted for 24 hours. A further quantity of reagent E from Example 27b was added to the reaction mixture (4.9 mg, 0.032 mmol) and the reaction continued for a further 44 hours. The product, 2-methoxy-6-(4-methoxybenzyloxy)iminoestrone-17-oxime, was obtained in a yield of 70%.

¹H NMR (CDCl₃): δ7.57 (s, 1H), 7.37 (d, J=8.5 Hz, 2H), 6.90 (d, J=8.5 Hz, 2H), 6.76 (s, 1H), 5.54 (br s, 1H), 5.12 (s, 2H), 3.91 (s, 3H), 3.81 (s, 3H), 3.16 (dd, J=18.0, 4.4 Hz, 1H), 2.62-1.39 (m, 12H), 0.92 (s, 3H); ¹³C NMR (CDCl₃) δ170.82, 159.26, 153.05, 147.74, 143.89, 134.82, 130.31, 130.05, 123.88, 113.69, 109.91, 106.72, 75.94, 55.84, 55.24, 53.19, 43.96, 41.80, 36.46, 33.55, 29.41, 25.55, 25.05, 22.90, 16.98; ESI-MS (20v) m/z 465 (100) [M+H]⁺; HRESI-MS: [M+H]⁺ 465.2382 (465.2390 calc.)

### Example 28

### Preparation of 2-methoxy-6-(4-methoxybenzyloxy)iminoestradiol

### CP-DM-4-5

The same procedure was used as outlined in Example 4c above with the following variations.

As reagent F, 2-methoxy-6-oxo-estradiol was used in an amount of 17.3 mg (0.055 mmol), reagent E from Example 27b was used in an amount of 10.5 mg (0.068 mmol), resin G was used in an amount of 86.6 mg, and resin H was used in an amount of 91.3 mg. The reaction was conducted for 24 hours. A further quantity of reagent E was added to the reaction mixture (3.5 mg, 0.022 mmol) and the reaction was continued for a further 40 hours. The product was purified by dissolution in methanol (8 mL) and filtration through a C18 cartridge (loading 300 mg). The product, 2-methoxy-6-(4-methoxybenzyloxy)iminoestradiol, was obtained in a yield of 80%.

¹H NMR (CDCl₃): δ7.56 (s, 1H), 7.36 (d, J=8.5 Hz, 2H), 6.89 (d, J=8.7 Hz, 2H), 6.77 (s, 1H), 5.51 (br s, 1H), 5.11 (s, 2H), 3.90 (s, 3H), 3.81 (s, 3H), 3.72 (t, J=8.3 Hz, 1H), 3.16 (dd, J=18.1, 4.5 Hz, 1H), 2.35-1.14 (m, 12H), 0.75 (s, 3H); ESI-MS (20v) m/z 452 (100) [M+H]⁺; HRESI-MS: [M+H]⁺ 452.2435 (452.2437 calc.)

### Example 291

### Preparation of 2-methoxy-6-(3-trifluoromethylbenzyloxy)imino-estrone-17-oxime (CP-DM-3-118)

This is prepared via two intermediates, as follows.

### Example 29a

### Preparation of N-(3-trifluoromethylbenzyloxy)phthalimide

The same procedure was used as outlined in Example 23a above with the following variations.

As reagent B, 3-trifluoromethylbenzyl bromide was used in 1.1019 g (7.214 mmol), reagent A was used in an amount of 1.070 g (6.559 mmol), and 2.246 mL (13.18 mmol) of reagent C was used. The reaction was conducted for 41 hours at ambient temperature. A further quantity of reagent B [3-trifluoromethylbenzyl bromide] (250.0 mg, 1.63 mmol) was added to the reaction mixture and the reaction continued for a further 48 hours at ambient temperature. The reaction was diluted with water (50 mL). Purification was performed by flash chromatography on silica gel [toluene then dichloromethane]. The product D, N-(3-trifluoromethylbenzyloxy)phthalimide, was obtained in a yield of 39%.

¹H NMR (CDCl₃): δ7.89-7.72 (m, 6H), 7.64 (d, J=7.8 Hz, 1H), 7.53 (dd, J=8.0, 7.6 Hz, 1H), 5.26 (s, 2H).

### Example 29b

### Preparation of O-(3-trifluoromethylbenzyl)hydroxylamine hydrochloride

The same procedure was used as outlined in Example 23b above with the following variations.

As reagent D, N-(3-trifluoromethylbenzyloxy)phthalimide was used in 588.0 mg (1.737 mmol), and reagent I was used in an amount of 101.3 μL (2.084 mmol). The reaction was conducted in ethanol (6 mL) and dichloromethane (3 mL) for 16 hours at reflux. Further quantities of ethanol (5 mL) and dichloromethane (2.5 mL) were added and the reaction continued for a further 5 hours at reflux. The product O-(3-trifluoromethylbenzyl)hydroxylamine hydrochloride (E) was obtained in a yield of 13%.

¹H NMR (CD₃OD): δ7.78-7.60 (m, 4H), 5.10 (s, 2H).

### Example 29c

### Preparation of 2-methoxy-6-(3-trifluoromethylbenzyloxy)imino-estrone-17-oxime (CP-DM-3-118)

The same procedure was used as outlined in Example 4c above with the following variations.

As reagent F, (2-methoxy-6-oxo-estrone-17-oxime) was used in an amount of 7.5 mg (0.023 mmol), reagent E from Example 29b was used in an amount of 6.5 mg (0.029 mmol), resin G was used in an amount of 80 mg, and resin H was used in an amount of 30 mg. The reaction was conducted for 66 hours. Filtration was through a LC-CN solid phase extraction cartridge. The product, 2-methoxy-6-(3-trifluoromethylbenzyloxy)iminoestrone-17-oxime was obtained in a yield of 95%.

¹H NMR (CDCl₃): δ7.66 (br s, 1H), 7.62-7.44 (m, 4H), 6.78 (s, 1H), 5.49 (br s, 1H), 5.23 (s, 2H), 3.91 (s, 3H), 3.20 (dd, J=18.0, 4.4 Hz, 1H), 2.62-1.16 (m, 12H), 0.94 (s, 3H); ESI-MS (20v) m/z 503 (100) [M+H]⁺; HRESI-MS: [M+H]⁺ 503.2155 (503.2158 calc.)

### Example 30

### Preparation of 6-(2,4-dinitrophenylhydrazono)-2-methoxy-estrone-17-oxime (CP-DM-3-119)

The same procedure was used as outlined in Example 4c above with the following variations.

As reagent F, 2-methoxy-6-oxo-estrone-17-oxime was used in an amount of 11.6 mg (0.035 mmol), reagent E (2,4-dinitrophenyl hydrazine) was used in an amount of 8.7 mg (0.044 mmol), resin G was used in an amount of 80 mg, and resin H was used in an amount of 35 mg. An amount of hydrochloric acid (37%, 100.0 μL) was added to the reaction. The reaction was conducted for 16 hours at ambient temperature. The product was purified by dissolution in methanol (8 mL) and filtration through a C18 cartridge (loading 300 mg). The product, 6-(2,4-dinitrophenylhydrazono)-2-methoxy-estrone-17-oxime, was obtained in a yield of 24%.

¹H NMR (CDCl₃): δ9.16 (d, J=2.1 Hz, 1H), 8.38 (dd, J=9.7, 2.1 Hz, 1H), 8.15 (d, J=9.7 Hz, 1H), 7.79 (s, 1H), 6.84 (s, 1H), 3.97 (s, 3H), 2.99 (dd, J=16.7, 4.9 Hz), 2.66-1.11 (m, 12H), 0.98 (s, 1H); ESI-MS (20v) m/z 508 (100) [M−H]⁻; HRESI-MS: [M−H] 508.1825 (508.1832 calc.)

### Example 31

### Preparation of estrone-17-(4-nitrobenzyl)oxime (CP-DM-3-101)

Estrone (16.0 mg, 0.059 mmol), O-(4-nitrobenzyl)hydroxylamine (30.3 mg, 0.148 mmol) and 4-polyvinylpyridine (25% cross-linked, 81.3 mg) were all added to reaction vessel under a nitrogen atmosphere and solvated with methanol (10 mL) with stirring. The reaction was conducted at ambient temperature for 24 hours followed by 24 hours at reflux. The reaction was evaporated in vacuo and PS-Benzaldehyde resin (222 mg, 1.20 mmolg⁻¹) added to vessel. The reaction mixture was suspended in anhydrous tetrahydrofuran (10 mL) and stirred at ambient temperature for 60 hours prior to filtration through a polyethylene frit which was further washed with 2×5 mL tetrahydrofuran. The combined filtrate and washings were evaporated to give a pale yellow solid which was suspended in methanol (5 mL). This suspension was passed through a C18 cartridge (300 mg loading) which was washed with a further quantity of methanol (2 mL). The combined filtrate and washings were evaporated to yield the product, estrone-17-(4-nitrobenzyl)oxime, in 86% yield.

¹H NMR (CDCl₃): δ8.20 (d, J=8.6 Hz, 2H), 7.49 (d, J=8.8 Hz, 2H), 7.14 (d, J=8.4 Hz, 1H), 6.62 (dd, J=8.4, 2.7 Hz, 1H), 6.56 (d, J=2.6 Hz, 1H), 5.17 (s, 2H), 4.93 (br s, 1H), 2.92-1.22 (m, 15H), 0.94 (s, 3H); ¹³C NMR (CDCl₃) δ171.89, 153.45, 147.26, 146.32, 138.01, 132.24, 127.99, 126.45, 123.48, 115.24, 112.74, 73.89, 52.92, 44.51, 43.91, 38.11, 34.09, 29.47, 27.17, 26.15, 22.97, 17.30; ESI-MS (20v) m/z 421 (100) [M+H]⁺; HRESI-MS: [M+H]⁺ 421.2122 (421.2127 calc.)

### Example 32

### Preparation of 2-methoxyestrone-17-(4-nitrobenzyl)oxime (CP-DM-3-103)

2-Methoxyestrone (9.8 mg, 0.033 mmol), O-(4-nitrobenzyl)hydroxylamine [commercially available] (16.7 mg, 0.082 mmol) and 4-polyvinylpyridine (25% cross-linked, 72.9 mg) were all added to reaction vessel under a nitrogen atmosphere and solvated with methanol (10 mL) with stirring. The reaction was conducted at ambient temperature for 24 hours followed by 16 hours at reflux. The reaction was evaporated in vacuo and PS-Benzaldehyde resin (122 mg, 1.20 mmolg⁻¹) added to vessel. The reaction mixture was suspended in anhydrous tetrahydrofuran (10 mL) and stirred at ambient temperature for 60 hours prior to filtration through a polyethylene frit which was further washed with 2×5 mL tetrahydrofuran. The combined filtrate and washings were evaporated to give a pale yellow solid which was suspended in methanol (5 mL). This suspension was passed through a C18 cartridge (300 mg loading) which was washed with a further quantity of methanol (2 mL). The combined filtrate and washings were evaporated to yield the product, 2-methoxyestrone-17-(4-nitrobenzyl)oxime, in 73% yield.

¹H NMR (CDCl₃): δ8.18 (d, J=8.7 Hz, 2H), 7.48 (d, J=8.6 Hz, 2H), 6.76 (s, 1H), 6.63 (s, 1H), 5.47 (br s, 1H), 5.16 (s, 1H), 3.84 (s, 3H), 2.88-1.22 (m, 15H), 0.93 (s, 3H); ¹³C NMR (CDCl₃) δ171.79, 147.23, 146.35, 144.60, 131.31, 129.26, 127.96, 123.47, 114.58, 107.94, 73.88, 56.03, 52.91, 44.46, 44.20, 38.09, 34.12, 28.84, 27.29, 26.44, 26.14, 22.94, 17.31; ESI-MS (20v) m/z 451 (100) [M+H]⁺; HRESI-MS: [M+H]⁺ 451.2222 (451.2233 calc.)

### Example 33

### Preparation of estrone-17-(3-nitrobenzyl)oxime (CP-DM-3-102)

The reagent O-(3-nitrobenzyl)hydroxylamine was prepared by the method of Examples 23a and 23b above.

Estrone (14.9 mg, 0.055 mmol), O-(3-nitrobenzyl)hydroxylamine [from Example 23b] (28.2 mg, 0.138 mmol) and 4-polyvinylpyridine (25% cross-linked, 91.5 mg) were all added to reaction vessel under a nitrogen atmosphere and solvated with methanol (10 mL) with stirring. The reaction was conducted at ambient temperature for 24 hours followed by 24 hours at reflux. The reaction was evaporated in vacuo and PS-Benzaldehyde resin (207 mg, 1.20 mmolg⁻¹) added to vessel. The reaction mixture was suspended in anhydrous tetrahydrofuran (10 mL) and stirred at ambient temperature for 60 hours prior to filtration through a polyethylene frit which was further washed with 2×5 mL tetrahydrofuran. The combined filtrate and washings were evaporated to give a pale yellow solid which was suspended in methanol (5 mL). This suspension was passed through a C18 cartridge (300 mg loading) which was washed with a further quantity of methanol (2 mL). The combined filtrate and washings were evaporated to yield the product, estrone-17-(3-nitrobenzyl)oxime, in 77% yield.

¹H NMR (CDCl₃): δ8.23 (br s, 1H), 8.14 (dd, J=8.3, 2.0 Hz, 1H), 7.66 (d, J=7.5 Hz, 1H), 7.51 (dd, J=7.9, 7.8 Hz, 1H), 7.14 (d, 8.5 Hz, 1H), 6.63 (dd, J=8.4, 2.8 Hz, 1H), 6.57 (d, J=2.7 Hz, 1H), 5.16 (s, 2H), 4.78 (br s, 1H), 2.92-1.22 (m, 15H), 0.94 (s, 3H); ¹³C NMR (CDCl₃) δ172.18, 153.61, 148.46, 141.10, 138.27, 133.86, 132.53, 129.34, 126.71, 122.84, 122.66, 115.44, 112.94, 74.07, 53.11, 44.74, 44.13, 38.32, 34.32, 29.71, 27.39, 26.40, 23.20, 17.51; ESI-MS (20v) m/z 421 (100) [M+H]⁺; HRESI-MS: [M+H]⁺ 421.2122 (421.2127 calc.)

### Example 34

### Preparation of 2-methoxyestrone-17-(3-nitrobenzyl)oxime (CP-DM-3-104)

The reagent O-(3-nitrobenzyl)hydroxylamine was prepared by the method of Examples 23a and 23b above.

2-Methoxyestrone (9.7 mg, 0.032 mmol), O-(3-nitrobenzyl)hydroxylamine [from Example 23b] (16.5 mg, 0.081 mmol) and 4-polyvinylpyridine (25% cross-linked, 63.3 mg) were all added to reaction vessel under a nitrogen atmosphere and solvated with methanol (10 mL) with stirring. The reaction was conducted at ambient temperature for 24 hours followed by 16 hours at reflux. The reaction was evaporated in vacuo and PS-Benzaldehyde resin (121 mg, 1.20 mmolg⁻¹) added to vessel. The reaction mixture was suspended in anhydrous tetrahydrofuran (10 mL) and stirred at ambient temperature for 60 hours prior to filtration through a polyethylene frit which was further washed with 2×5 mL tetrahydrofuran. The combined filtrate and washings were evaporated to give a pale yellow solid which was suspended in methanol (5 mL). This suspension was passed through a C18 cartridge (300 mg loading) which was washed with a further quantity of methanol (2 mL). The combined filtrate and washings were evaporated to yield the product, 2-methoxyestrone-17-(3-nitrobenzyl)oxime, in 76% yield.

¹H NMR (CDCl₃): δ8.24 (s, 1H), 8.15 (dd, J=8.8, 2.1 Hz, 1H), 7.66 (d, J=7.6 Hz, 1H), 7.51 (dd, J=8.0, 7.8 Hz, 1H), 7.14 (d, 8.5 Hz, 1H), 6.78 (s, 1H), 6.65 (s, 1H), 5.45 (br s, 1H), 5.16 (s, 2H), 3.86 (s, 3H), 2.94-1.24 (m, 15H), 0.95 (s, 3H); ESI-MS (20v) m/z 451 (100) [M+H]⁺; HRESI-MS: [M+H]⁺ 451.2214 (451.2233 calc.)

### Example 35

### Preparation of estrone-17-(4-methoxybenzyl)oxime (CP-DM-4-33)

The reagent O-(4-methoxybenzyl)hydroxylamine was prepared by the method of Examples 27a and 27b above.

Estrone (4.2 mg, 0.016 mmol), O-(4-methoxybenzyl)hydroxylamine [from Example 27b] (4.4 mg, 0.023 mmol) and 4-polyvinylpyridine (25% cross-linked, 101.9 mg) were all added to reaction vessel under a nitrogen atmosphere and solvated with methanol (10 mL) with stirring. The reaction was conducted at reflux for 48 hours. The reaction was evaporated in vacuo and PS-Benzaldehyde resin (44 mg, 1.20 mmolg⁻¹) added to vessel. The reaction mixture was suspended in anhydrous tetrahydrofuran (10 mL) and stirred at ambient temperature for 70 hours prior to filtration through an LC-C18 solid phase extraction cartridge which was further washed with 4×5 mL tetrahydrofuran. The combined filtrate and washings were evaporated to yield the product, estrone-17-(4-methoxybenzyl)oxime, in 94% yield.

¹H NMR (DMSO-d6): δ9.00 (s, 1H), 7.26 (d, J=8.5 Hz, 2H), 7.03 (d, J=8.6 Hz, 1H), 6.89 (d, J=8.6 Hz, 2H), 6.49 (d, J=8.4 Hz, 1H), 6.42 (s, 1H), 4.90 (s, 2H), 3.73 (s, 3H), 2.77-1.16 (m, 15H), 0.86 (s, 3H); ESI-MS (20v) m/z 406 (100) [M+H]⁺; HRESI-MS: [M+H]⁺ 406.2377 (406.2382 calc.)

### Example 36

### Preparation of 2-methoxyestrone-17-(4-methoxybenzyl)oxime (CP-DM-4-34)

The reagent O-(4-methoxybenzyl)hydroxylamine was prepared by the method of Examples 27a and 27b above.

2-Methoxyestrone (4.6 mg, 0.015 mmol), O-(4-methoxybenzyl)hydroxylamine [from Example 27b] (4.4 mg, 0.023 mmol) and 4-polyvinylpyridine (25% cross-linked, 98.6 mg) were all added to reaction vessel under a nitrogen atmosphere and solvated with methanol (10 mL) with stirring. The reaction was conducted at reflux for 48 hours. The reaction was evaporated in vacuo and PS-Benzaldehyde resin (50 mg, 1.20 mmolg⁻¹) added to vessel. The reaction mixture was suspended in anhydrous tetrahydrofuran (10 mL) and stirred at ambient temperature for 70 hours prior to filtration through an LC-C18 solid phase extraction cartridge which was further washed with 4×5 mL tetrahydrofuran. The combined filtrate and washings were evaporated to yield the product, 2-methoxyestrone-17-(4-methoxybenzyl)oxime, in 88% yield.

¹H NMR (DMSO-d6): δ8.56 (br s, 1H), 7.26 (d, J=8.2 Hz, 2H), 6.89 (d, J=8.4 Hz, 2H), 6.76 (s, 1H), 6.44 (s, 1H), 4.91 (s, 2H), 3.74 (s, 3H), 3.71 (s, 3H) 2.72-1.13 (m, 15H), 0.87 (s, 3H); ESI-MS (20v) m/z 436 (100) [M+H]⁺; HRESI-MS: [M+H]⁺ 436.2491 (436.2488 calc.)

### Example 37

### Preparation of 6-(3,5-difluorobenzyloxy)iminoestriol (CP-DM-4-88)

The reagent O-(3,5-difluorobenzyl)hydroxylamine was prepared by the method of Examples 4a and 4b above.

6-Oxo-estriol (8.4 mg, 0.028 mmol), O-(3,5-difluorobenzyl)hydroxylamine [from Example 4b] (10.9 mg, 0.056 mmol) and 4-polyvinylpyridine (25% cross-linked, 150 mg) were all added to reaction vessel under a nitrogen atmosphere and solvated with methanol (10 mL) with stirring. The reaction was conducted at reflux for 48 hours. The reaction was evaporated in vacuo and PS-Benzaldehyde resin (69.7 mg, 1.20 mmolg⁻¹) added to vessel. The reaction mixture was suspended in anhydrous tetrahydrofuran (10 mL) and stirred at ambient temperature for 2.5 hours prior to filtration through an LC-CN solid phase extraction cartridge which was further washed with 3×5 mL tetrahydrofuran. The combined filtrate and washings were evaporated to yield a white solid which was purified by flash chromatography on silica gel [ethyl acetate-petroleum spirit (40-60° C.) (2:1)] the product, 6-(3,5-difluorobenzyloxy)iminoestriol, in 86% yield.

¹H NMR (CD₃OD/CDCl₃): δ7.31 (d, J=2.7 Hz, 1H), 7.13 (d, J=8.6 Hz, 1H), 6.92-6.86 (m, 2H), 6.77 (dd, J=8.4, 2.7 Hz, 1H), 6.73 (tt, J=6.8, 2.2 Hz, 1H), 5.14 (s, 2H), 4.08-4.02 (m, 1H), 3.46 (d, J=5.9 Hz, 1H), 3.08 (dd, J=18.2, 4.3 Hz, 1H), 2.30-1.23 (m, 10H), 0.73 (s, 3H); ESIMS (20v) m/z 444 (100) [M+H]⁺.

### Example 38

### Preparation of 2-Methoxy-6-(4-nitrobenzyloxy)iminoestradiol-17-acetate (CP-DM-4-91)

2-Methoxyestradiol-17-acetate (16.3 mg, 0.045 mmol), O-(4-nitrobenzyl)hydroxylamine (18.6 mg, 0.091 mmol) and 4-polyvinylpyridine (25% cross-linked, 150 mg) were all added to reaction vessel under a nitrogen atmosphere and solvated with methanol (10 mL) with stirring. The reaction was conducted at reflux for 48 hours. The reaction was evaporated in vacuo and PS-Benzaldehyde resin (113.6 mg, 1.20 mmolg⁻¹) added to vessel. The reaction mixture was suspended in anhydrous tetrahydrofuran (10 mL) and stirred at ambient temperature for 2.5 hours prior to filtration through an LC-CN solid phase extraction cartridge which was further washed with 3×5 mL tetrahydrofuran. The combined filtrate and washings were evaporated to yield the product, 2-Methoxy-6-(4-nitrobenzyloxy)iminoestradiol-17-acetate, in 92% yield.

¹H NMR (CDCl₃): δ8.21 (d, J=8.4 Hz, 2H), 7.53 (d, J=8.6 Hz, 2H), 7.46 (s, 1H), 6.76 (s, 1H), 5.49 (br s, 1H), 5.26 (s, 2H), 4.69 (t, J=8.5 Hz, 1H), 3.90 (s, 3H), 3.15 (dd, J=18.1, 4.5 Hz, 1H), 2.28-1.31 (m, 12H), 2.07 (s, 3H), 0.81 (s, 3H); ESIMS (20v) m/z 509 (100) [M+H]⁺.

### Example 39

### Preparation of 2-methoxy-6-(4-nitrobenzyloxy)imino-estrone-17-methyloxime (CP-DM-4-75)

This is prepared via four intermediates, as follows.

### Example 39a

### Preparation of 2-methoxyestrone-17-methyloxime

2-Methoxyestrone (98.3 mg, 0.327 mmol) was dissolved in methanol (12 mL) and methoxylamine hydrochloride (82.0 mg, 0.981 mmol) and 4-polyvinylpyridine (25% cross-linked, 207.0 mg) added with stirring under a nitrogen atmosphere. The reaction was conducted at reflux for 21 hours prior to evaporation in vacuo. The mixed solid was suspended in dichloromethane (10 mL) and filtered through a LC-CN solid phase extraction cartridge which was washed with a further 5 mL of dichloromethane. Evaporation in vacuo yielded an amorphous white solid in a yield of 99%.

¹H NMR (CDCl₃): δ6.90 (s, 1H), 6.74 (s, 1H), 3.84 (s, 3H), 3.81 (s, 3H), 2.81-1.37 (m, 15H), 0.94 (s, 3H); ESI-MS (20v) m/z 330 (100) [M+H]⁺; HRESI-MS: [M+H]⁺ 330.2082 (330.2069 calc.)

### Example 39b

### Preparation of 2-methoxyestrone-17-methyloxime-3-acetate

2-methoxyestrone-17-methyloxime (106.5 mg, 0.323 mmol) was dissolved in pyridine (2.0 mL, 24.80 mmol) and acetic anhydride (1.0 mL, 10.58 mmol) was added dropwise with stirring under a nitrogen atmosphere at ambient temperature. After 15.5 hours, the reaction was diluted with aqueous 1 M hydrochloric acid solution prior to extraction with 3×10 mL ethyl acetate. Combined extracts were washed with water (10 mL), and brine (10 mL) prior to drying over anhydrous sodium sulphate. Filtration and evaporation in vacuo gave an off white solid in a yield of 90%.

¹H NMR (CDCl₃): δ6.89 (s, 1H), 6.74 (s, 1H), 3.85 (s, 3H), 3.81 (s, 3H), 2.81-1.33 (m, 15H), 2.29 (s, 3H), 0.93 (s, 3H); ESI-MS (20v) m/z 372 (100) [M+H]⁺.

### Example 39c

### Preparation of 2-methoxy-6-oxo-estrone-17-methyloxime-3-acetate

Chromium Trioxide (119.5 mg, 1.195 mmol) was dissolved in an acetic acid (1.80 mL)and water (0.20 mL) mixture. A solution of 2-methoxyestrone-17-methyloxime-3-acetate (104.5 mg, 0.281 mmol) in acetic acid (2.00 mL) was added followed by further amounts of acetic acid (1.80 mL) and water (0.20 mL) and the reaction held at 5-8° C. for 50 minutes. The reaction was diluted with water (25 mL) prior to extraction with 3×20 mL ethyl acetate. Combined extracts were washed with saturated sodium hydrogen carbonate solution (2×20 mL), water (20 mL), and brine (20 mL) prior to drying over anhydrous sodium sulphate. Filtration and evaporation in vacuo gave a white solid in a yield of 87%.

¹H NMR (CDCl₃): δ7.75 (s, 1H), 6.95 (s, 1H), 3.91 (br s, 3H), 3.85 (s, 3H), 2.77 (dd, J=16.8, 3.4 Hz, 1H), 2.62-1.25 (m, 12H), 2.32 (s, 3H), 0.96 (s, 3H); ESI-MS (20v) m/z 386 (100) [M+H]⁺.

### Example 39d

### Preparation of 2-methoxy-6-oxo-estrone-17-methyloxime

2-Methoxy-6-oxo-estrone-17-methyloxime-3-acetate (84.4 mg, 0.219 mmol) was dissolved in ethanol (10.0 mL) and potassium carbonate (934 mg, 6.758 mmol) added with stirring under a nitrogen atmosphere. Stirring continued at ambient temperature for 18 hours prior to addition of 1 M ammonium chloride solution (25.0 mL). The pH of the reaction was further adjusted to pH=7 by dropwise addition of 6 M hydrochloric acid solution. Extraction was performed with dichloromethane (3×20 mL). Combined extracts were washed with water (20 mL) and brine (20 mL) prior to drying over over anhydrous sodium sulphate. Filtration and evaporation in vacuo gave the crude product. Purification was performed by flash chromatography on silica gel [ethyl acetate-petroleum spirit (40-60° C.) (1:2)]. The product was obtained as a white solid in a yield of 78%.

¹H NMR (CDCl₃): δ7.59 (s, 1H), 6.83 (s, 1H), 5.52 (br s, 1H), 3.96 (s, 3H), 3.83 (s, 3H), 2.73 (dd, J=16.8, 3.5 Hz, 1H), 2.56-1.37 (m, 12H), 0.94 (s, 3H); ESI-MS (20v) m/z 344 (100) [M+H]⁺, 366 (64) [M+Na]⁺; HRESI-MS: [M+H]⁺ 344.1851 (344.1826 calc.).

### Example 39e

### Preparation of 2-methoxy-6-(4-nitrobenzyloxy)iminoestrone-17-methyloxime (CP-DM-4-75)

2-Methoxy-6-oxo-estrone-17-methyloxime (16.4 mg, 0.048 mmol) and O-(4-nitrobenzyl)hydroxylamine hydrochloride (12.7 mg, 0.062 mmol) were dissolved in methanol (10 mL) with stirring under a nitrogen atmosphere. A quantity of 4-polyvinylpyridine (25% cross-linked, 217.4 mg) was added and the reaction was conducted at ambient temperature for 66 hours. The mixture was evaporated in vacuo prior to addition of PS-Benzaldehyde resin (39 mg, 1.20 mmolg⁻¹) and salvation with tetrahydrofuran (10 mL). The reaction mixture was stirred for a further 48 hours prior to filtration through a LC-CN solid phase extraction cartridge which was washed with a further 3×5 mL tetrahydrofuran. Combined filtrate and washings were evaporated to give an oily yellow residue that was purified by flash chromatography on silica gel [ethyl acetate-petroleum spirit (40-60° C.) (3:7)] to give the product in 66% yield.

¹H NMR (CDCl₃): δ8.21 (d, J=8.7 Hz, 2H), 7.54 (d, J=8.6 Hz, 2H), 7.47 (s, 1H), 6.78 (s, 1H), 5.50 (br s, 1H), 5.27 (s, 2H), 3.91 (s, 3H), 3.84 (s, 3H), 3.22 (dd, J=18.1, 4.5 Hz, 1H), 2.58-1.25 (m, 12H), 0.93 (s, 3H); ¹³C NMR (CDCl₃) δ169.65, 154.22, 148.03, 147.36, 146.11, 143.89, 135.20, 128.34, 127.83, 123.58, 123.22, 109.79, 106.77, 74.65, 55.85, 53.19, 43.85, 41.82, 36.49, 33.68, 29.46, 25.60, 22.97, 17.10; ESI-MS (20v) m/z 494 (100) [M+H]⁺; HRESI-MS: [M+H]⁺ 494.2288 (494.2291 calc.)

### Example 40

### Preparation of 2-methoxy-6-(3,5-difluorobenzyloxy)iminoestrone-17-methyloxime (CP-DM-4-74)

The two reagents used in this Example, 2-Methoxy-6-oxo-estrone-17-methyloxime and O-(3,5-difluorobenzyl)hydroxylamine hydrochloride, were prepared by the procedures of Examples 39a-39d and 4a-4-b, respectively.

2-Methoxy-6-oxo-estrone-17-methyloxime [from Example 39b] (14.5 mg, 0.042 mmol) and O-(3,5-difluorobenzyl)hydroxylamine hydrochloride [from Example 4b] (10.7 mg, 0.055 mmol) were dissolved in methanol (10 mL) with stirring under a nitrogen atmosphere. A quantity of 4-polyvinylpyridine (25% cross-linked, 227.8 mg) was added and the reaction was conducted at ambient temperature for 66 hours. The mixture was evaporated in vacuo prior to addition of PS-Benzaldehyde resin (39 mg, 1.20 mmolg⁻¹) and solvation with tetrahydrofuran (10 mL). The reaction mixture was stirred for a further 48 hours prior to filtration through a LC-CN solid phase extraction cartridge which was washed with a further 3×5 mL tetrahydrofuran. Combined filtrate and washings were evaporated to give an oily yellow residue that was purified by flash chromatography on silica gel [ethyl acetate-petroleum spirit (40-60° C.) (2:7)] to give the product in 52% yield.

¹H NMR (CDCl₃): δ7.50 (s, 1H), 6.93-6.87 (m, 2H), 6.78 (s, 1H), 6.72 (tt, J=9.0, 2.4 Hz, 1H), 5.48 (br s, 1H), 5.15 (s, 2H), 3.92 (s, 3H), 3.85 (s, 3H), 3.20 (dd, J=18.0, 4.6 Hz, 1H), 2.60-1.25 (m, 12H), 0.93 (s, 3H); ESI-MS (20v) m/z 485 (100) [M+H]⁺; HRESI-MS: [M+H]⁺ 485.2246 (485.2252 calc.)

### Example 40A

### Preparation of 6-(4-nitrobenzyloxy)imino-17-ethinyl-estradiol (CP-DM-4-89)

17-ethinyl-6-oxo-estradiol (8.9 mg, 0.029 mmol) and O-(3,5-4-nitrobenzyl)hydroxylamine (11.7 mg, 0.057 mmol) and 4-polyvinylpyridine (25% cross-linked, 150 mg) were all added to reaction vessel under a nitrogen atmosphere and solvated with methanol (10 mL) with stirring. The reaction was conducted at reflux for 48 hours. The reaction was evaporated in vacuo and PS-Benzaldehyde resin (71.7 mg, 1.20 mmolg⁻¹) added to vessel. The reaction mixture was suspended in anhydrous tetrahydrofuran (10 mL) and stirred at ambient temperature for 2.5 hours prior to filtration through an LC-CN solid phase extraction cartridge which was further washed with 3×5 mL tetrahydrofuran. The combined filtrate and washings were evaporated to yield a white solid which was purified by flash chromatography on silica gel [ethyl acetate-petroleum spirit (40-60° C.) (1:2)] the product, 6-(4-nitrobenzyloxy)imino-17-ethinyl-estradiol in 43% yield.

¹H NMR (CDCl₃): δ8.22 (d, J=8.6 Hz, 2H), 7.53 (d, J=8.6 Hz, 2H), 7.35 (d, J=2.9 Hz, 1H), 7.21 (d J=8.5 Hz, 1H), 6.84 (dd J=8.5, 2.9 Hz, 1H), 5.29 (s, 2H), 4.84 (br s, 1H), 2.62 (s, 1H), 3.14 (dd, J=18.3, 4.6 Hz, 1H), 2.40-1.24 (m, 12H), 0.86 (s, 3H); ESIMS (20v) m/z 461 (100) [M+H]⁺.

### Compounds of Examples 4-40A

The compounds of Examples 4-40A are represented below. 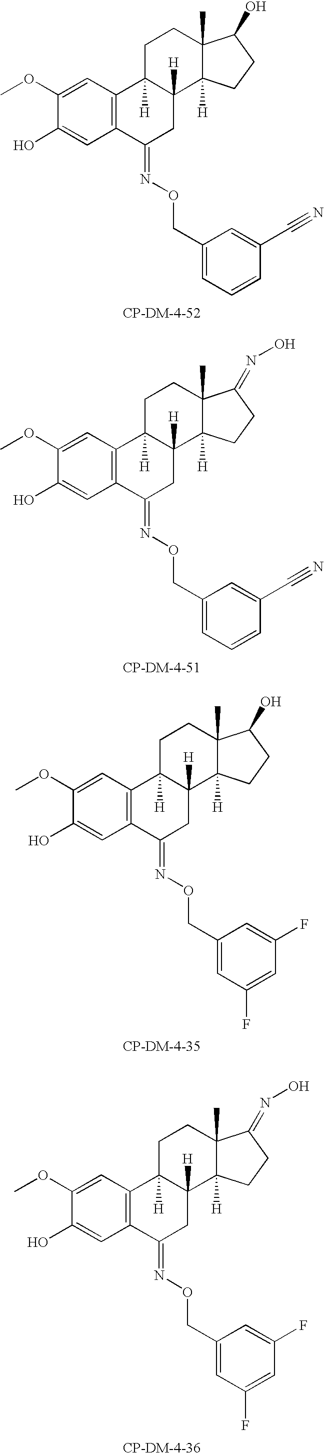 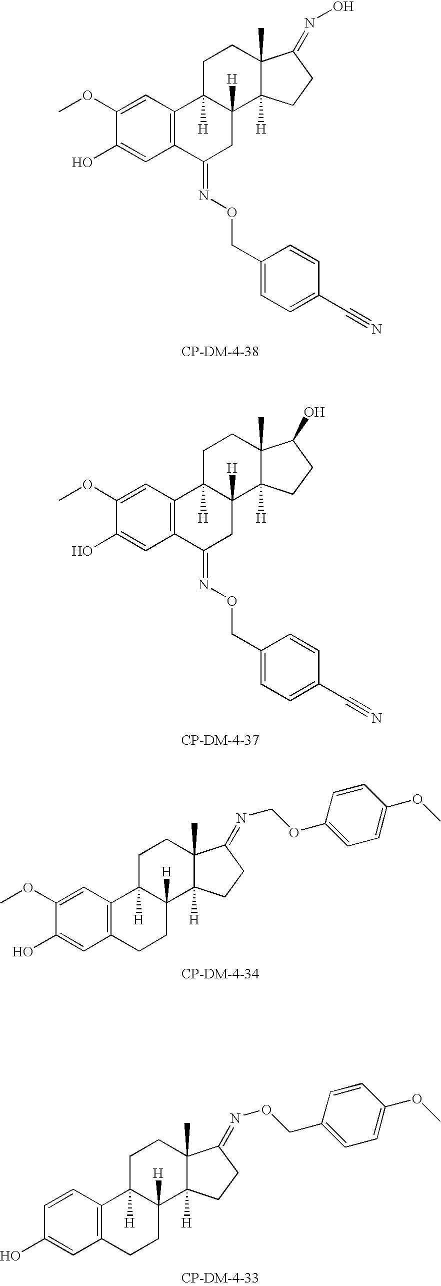 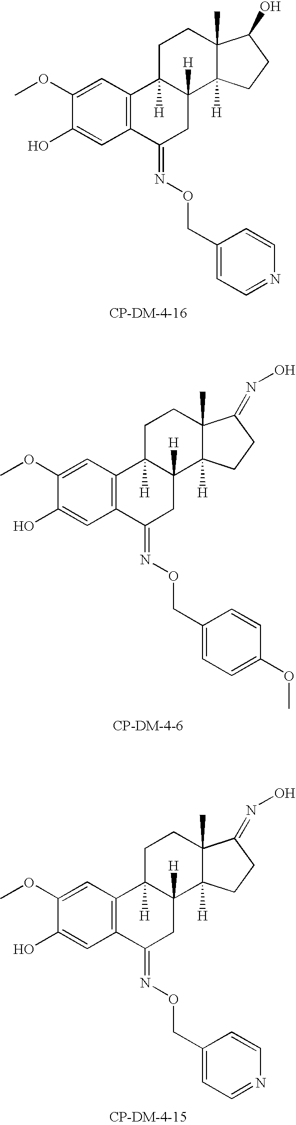 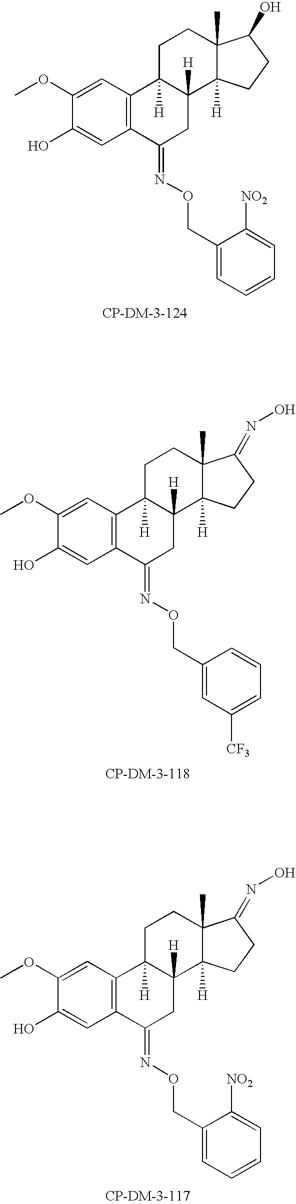 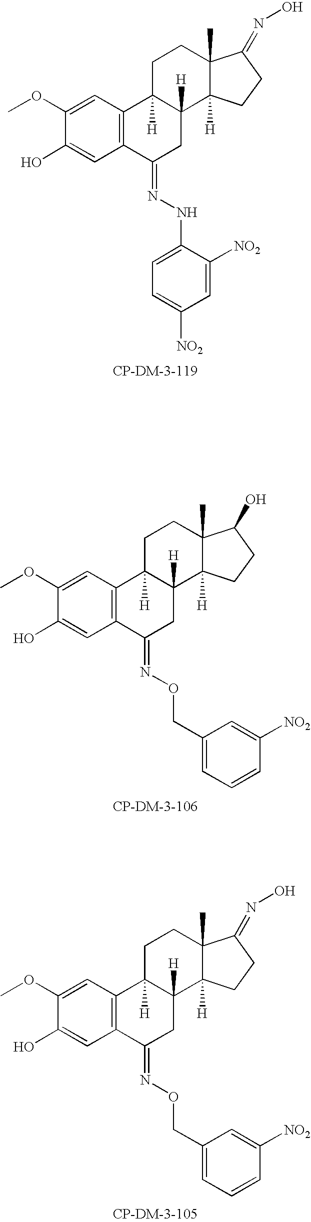 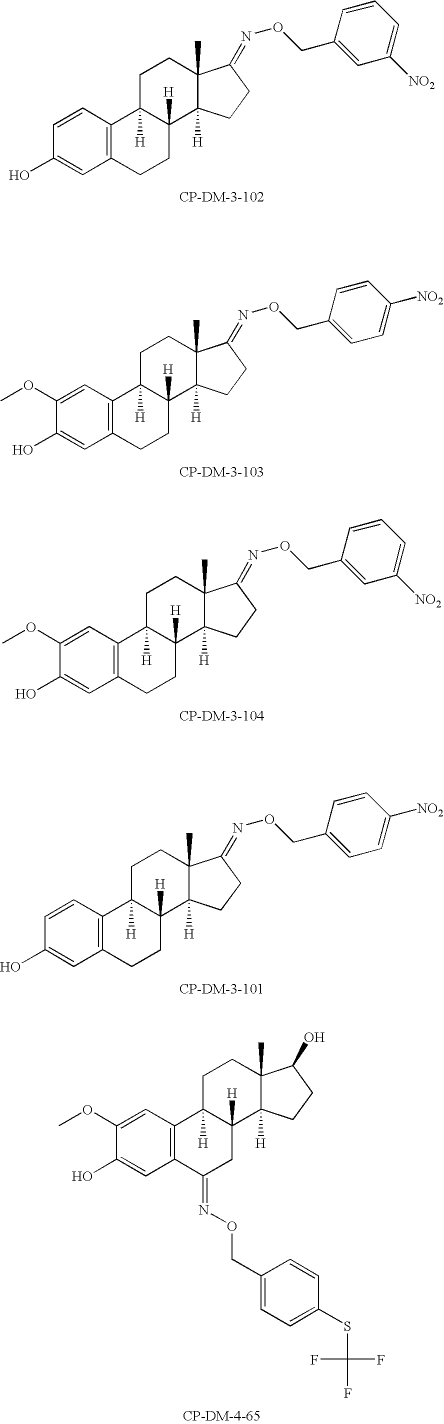 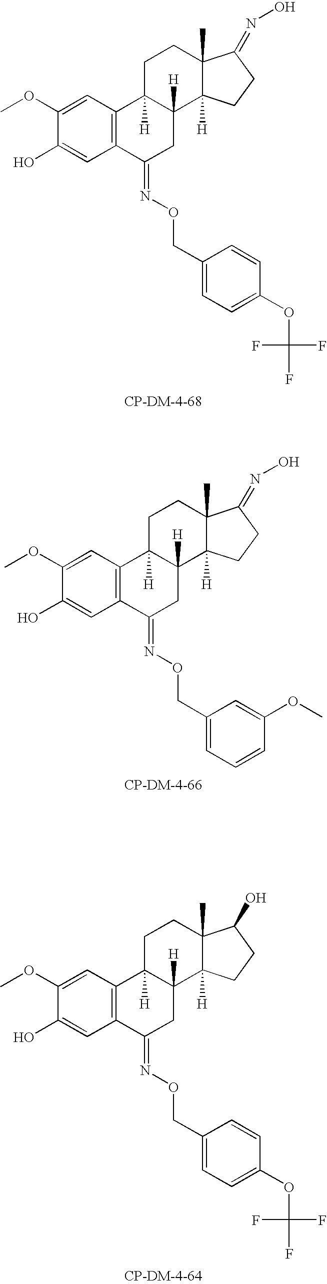 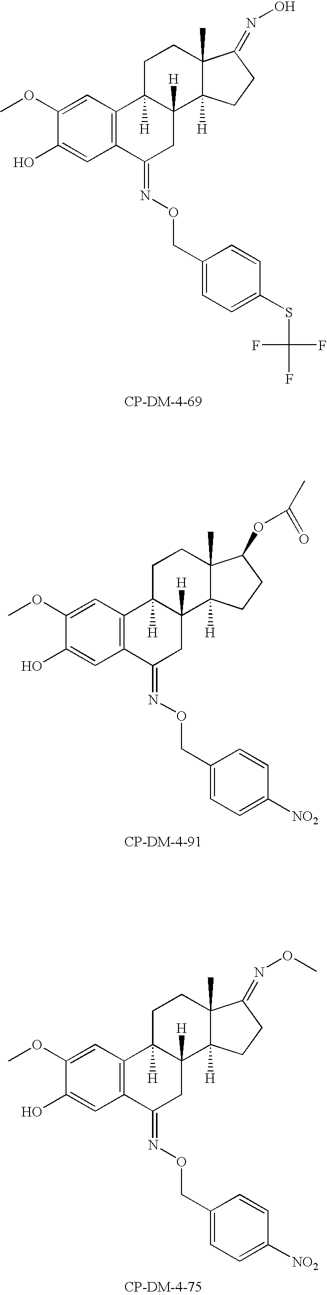 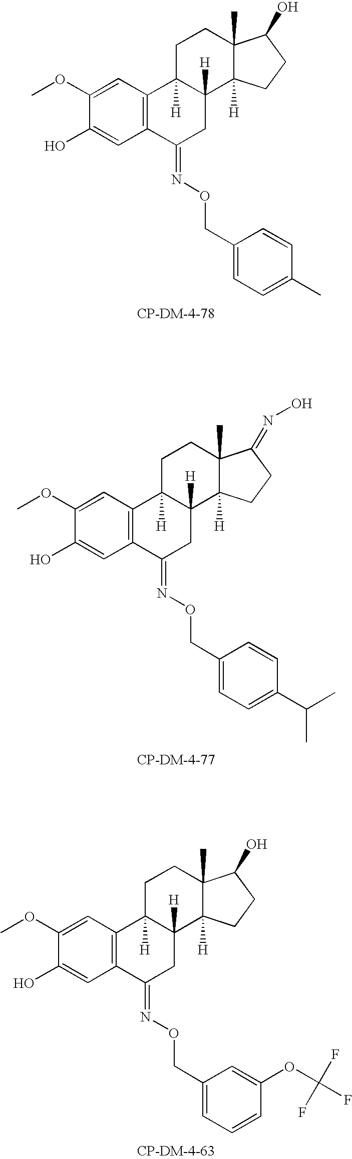 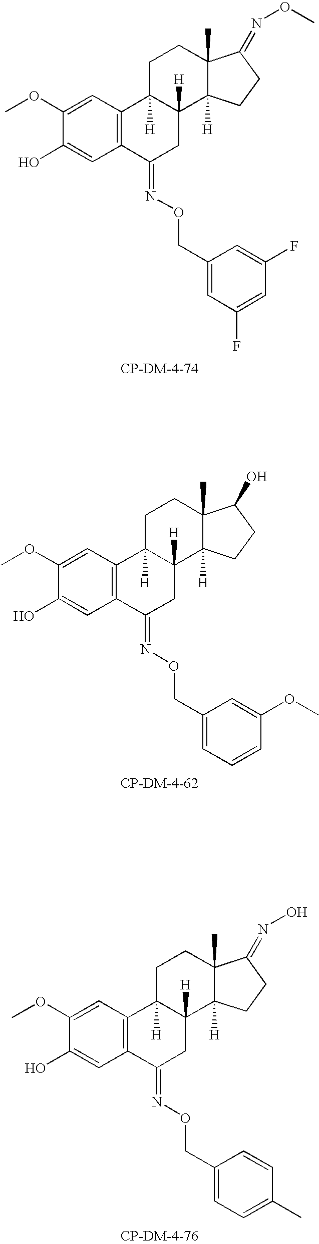 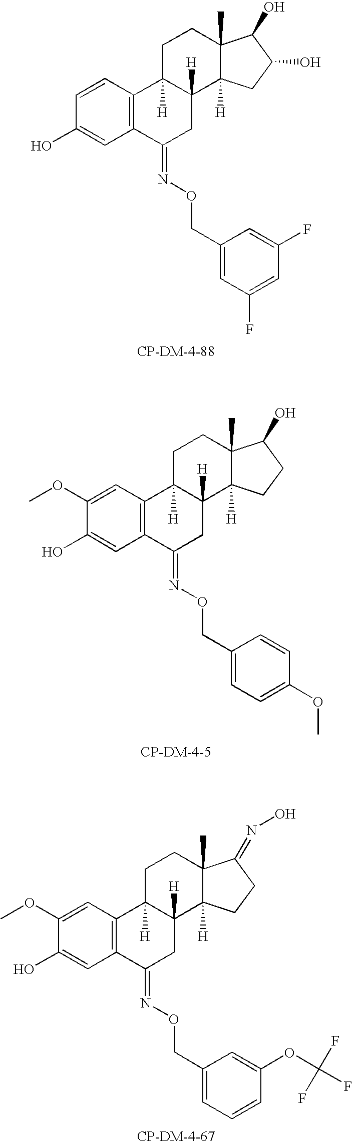 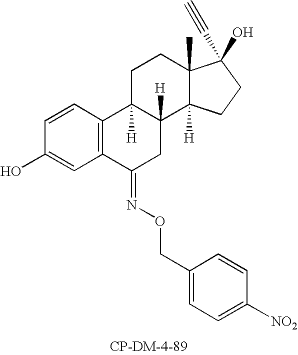
Synthesis of Other Compounds

The following compounds have been or can be synthesised by similar methods to those set out above:
2-methoxy-6-(3,5-difluorobenzyloxy)iminoestrone-17-oxime
2-methoxy-6-(2-cyanobenzyloxy)iminoestrone-17-oxime
2-methoxy-6-(3-cyanobenzyloxy)iminoestrone-17-oxime
2-methoxy-6-(4-cyanobenzyloxy)iminoestrone-17-oxime
2-methoxy-6-(2-nitrobenzyloxy)iminoestrone-17-oxime
2-methoxy-6-(3-nitrobenzyloxy)iminoestrone-17-oxime
2-methoxy-6-(3,5-difluorobenzyloxy)iminoestradiol
2-methoxy-6-(2-cyanobenzyloxy)iminoestradiol
2-methoxy-6-(3-cyanobenzyloxy)iminoestradiol
2-methoxy-6-(4-cyanobenzyloxy)iminoestradiol
2-methoxy-6-(2-nitrobenzyloxy)iminoestradiol
2-methoxy-6-(3-nitrobenzyloxy)iminoestradiol
2-methoxy-6-(2-nitrophenyloxy)iminoestradiol
2-methoxy-6-(3-nitrophenyloxy)iminoestradiol
2-methoxy-6-(2-nitrophenyloxy)iminoestrone-17-oxime
2-methoxy-6-(3-nitrophenyloxy)iminoestrone-17-oxime
2-methoxy-6-(2-pyridyloxy)iminoestradiol
2-methoxy-6-(3-pyridyloxy)iminoestradiol
2-methoxy-6-(4-pyridyloxy)iminoestradiol
2-methoxy-6-(2-pyridyloxy)iminoestrone-17-oxime
2-methoxy-6-(3-pyridyloxy)iminoestrone-17-oxime
2-methoxy-6-(4-pyridyloxy)iminoestrone-17-oxime
2-methoxyestrone-17-(4-nitrobenzyl)oxime
2-methoxyestrone-17-(3-nitrobenzyl)oxime
6-(4-cyanobenzyloxy)iminoestradiol
6-(4-nitrobenzyloxy)iminoestradiol
2-methoxy-6-(4-carboxybenzyloxy)iminoestradiol
2-methoxy-6-(4-methoxybenzyloxy)iminoestradiol
2-methoxy-6-(4-trifluoromethylbenzyloxy)iminoestrone-17-oxime
2-methoxy-6-(3-trifluoromethylbenzyloxy)iminoestrone-17-oxime
2-methoxy-6-(2-trifluoromethylbenzyloxy)iminoestrone-17-oxime
2-methoxy-6-(4-trifluoromethylbenzyloxy)iminoestradiol
2-methoxy-6-(3-trifluoromethylbenzyloxy)iminoestradiol
2-methoxy-6-(2-trifluoromethylbenzyloxy)iminoestradiol

### Example 41

### Studies on the Effectiveness of the Compounds of the Invention

Effects of Test Compounds on Human Airway Smooth Muscle Cell Proliferation

(a) Culture of Human Airway Smooth Muscle

Human airway smooth muscle (HASM) cells were cultured from macroscopically normal bronchi (0.5-2 cm diameter) obtained from lung resection or heart-lung transplant specimens provided by the Alfred Hospital (Melbourne) according to methods published in detail previously (Fernandes et al., 1999).

Approximately 0.1 g of smooth muscle was stripped from the wall of the bronchus for each cell culture. Dissected tissue was immersed in Dulbecco's modified Eagle's medium (DMEM) (Flow Laboratories, Scotland), supplemented with 100 U/mL penicillin G (CSL, Australia) and 100 ug/mL streptomycin (CSL, Australia). The tissue was rinsed in phosphate buffered saline (PBS; Oxoid, England) and the airway smooth muscle was chopped into 2 mm³ pieces and digested for 2 hours in DMEM containing elastase (0.5 mg/mL: Worthington Biochemical, USA) followed by a 12 hour incubation in DMEM containing collagenase (1 mg/mL) (Worthington Biochemical, USA), at 37° C. with agitation.

The resulting cell suspension was centrifuged and washed three times in phosphate buffered saline (PBS). Following the last centrifugation step, the cells were resuspended in 25 mL of DMEM supplemented with L-glutamine (2 mM: Sigma, USA), penicillin G (100 U/mL), streptomycin (100 μg/mL), amphotericin B (2 μg/mL: Wellcome, UK) and heat-inactivated FCS (10% v/v: CSL, Australia) and seeded into 25 cm² culture flasks. The primary isolates were incubated for 7 to 14 days to reach confluence.

Cells were harvested weekly by 10 min exposure to trypsin (0.5%: CSL, Australia) and EDTA (1 mM in PBS: BDH, Australia) and passaged at a 1:3 ratio into 75 cm² flasks.

The activity of the compounds has been established in cell culture by exploring their effects on mitogen-induced increases in cell number. We investigated the effects of 2MEO and its analogues, 4NO and 4NOM, on the proliferation of HASM cells in response to the proliferative stimulus of either thrombin of basic fibroblast growth factor (bFGF). Cells were seeded onto 6-well plates at a density of 1.5×10⁴ cells cm², made quiescent by removal of serum-containing media for 24 hours and then stimulated for 48 h with either thrombin (0.3 U/ml) or bFGF (300 pM). The test compounds were pre-incubated with HASM cells for 30 minutes before the addition of thrombin. At the end of the 48 hour incubation period, cells were detached from the culture plate by trypsin (0.5% w/v in PBS containing 1 mM EDTA), incubated for 5 minutes at ambient temperature in PBS containing 0.5% (w/v) trypan blue, washed twice (2% FCS in PBS), isolated by centrifugation (12,000×g, 5 min) and resuspended in 200 μl 2% FCS in PBS for counting in a haemocytometer chamber.

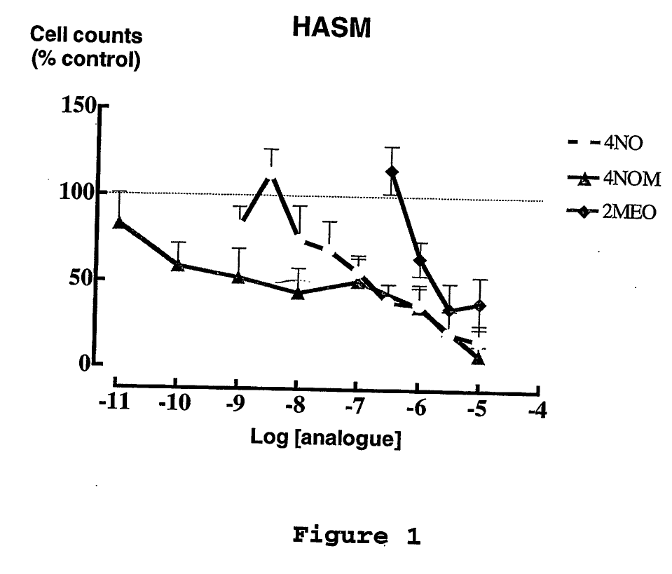

Results of these experiments are provided in FIG. 1 and Table 3. Data are expressed as a percentage of the cell number in vehicle treated wells and are presented as the mean and standard error of the mean.

TABLE 3
Effects of 2MEO analogues on proliferative responses of Human
Airway Smooth Muscle cells stimulated with thrombin or bFGF.

| | |
|---|---|
| Analogue | Cell number (% control) |

| (300 pM) | Thrombin (0.3 U/ml) | bFGF |
|---|---|---|
| — | 133.5 ± 4.1 (24) | 135.8 ± 3.7 (14) |
| 2MEO (10 μM) | 100.9 ± 8.1 (13) | 87.5 ± 13.8 (4) |
| 4NO (1 μM) | 111.1 ± 6.9 (13) | 116.6 ± 6.6 (14) |
| 4NOM (1 μM) | 117.5 ± 8.5 (8) | 111.1 ± 10.9 (4) |

The 2-methoxy-6-(4-nitrobenzyloxy)iminoestradiol (4NO) analogue shows inhibitory activity on proliferation of HASM cells over a wide concentration range (10 nM to 10 μM) (FIG. 1). Surprisingly, a related analogue of 2MEO, 2-Methoxy-6-(4-nitrobenzyloxy)iminoestrone-17-oxime (4NOM) was considerably more potent than either 2MEO or 4NO (FIG. 1). The inhibitory activity of 4NO was similar with either thrombin or basic Fibroblast Growth Factor (bFGF) as the mitogen (Table 3). The lack of selectivity between these two distinct mitogens suggests that this and related analogues are unlikely to work at the level of receptors for specific mitogens, but rather to influence intracellular signalling pathways that subserve such responses.

The effects of other disclosed compounds on the inhibition of thrombin-induced human airway smooth muscle cell proliferation were also tested using this assay, and the results are summarized in Columns A to C of Table 4.

The potency of the compounds in regulating thrombin-induced proliferation of human airway smooth muscle (HASM) was assessed in cells maintained in culture as described above. The potencies are presented as pIC₅₀ values (Column A of Table 4) denoting the negative log of the concentration that reduces the proliferative response by 50%. In addition, as the inhibitory effects of some compounds were biphasic, the data were also fitted to “two site” model in which the potency of compounds for the first phase corresponding to inhibition up to 50% and the second phase, inhibition up to 100% were determined and are presented pIC₂₅ and pIC₇₅ values (Columns B and C respectively). When the single phase model was determined to be more appropriate, the values of pIC25 and pIC75 were not calculated or shown in the table.

The data reveal that substitution on the benzyloximino ring attached to C6 of the B ring results in compounds that generally show significantly higher potency in regulation of proliferation than 2-methoxyestradiol. A group of compounds having similar benzyloximino substituents on the 17 position showed lesser potency than the corresponding 6-position analogues. The potency of this class of agents in regulating smooth muscle proliferation supports their utility in treating diseases characterised by smooth muscle proliferation.

Experiments were also carried out with bovine aortic vascular smooth muscle with a more limited set of compounds. At 3 μM 2-methoxyestradiol, CP-DM-2-11-7, and CP-DM-3-91 reduced bFGF (300 pM) induced proliferation by 87±6%, 75±14% and 93±4, respectively. These observations suggest that disclosed compounds may have therapeutic actions in diseases involving vascular smooth muscle dysfunction in the cardiovascular system.

It will be understood that such assays could be readily utilized to determine the ability of disclosed compounds to inhibit Thrombin or bFGF-induced proliferation of human airway smooth muscle cells. These assays may also be used to test the ability of such compounds to inhibit the Thrombin or bFGF-induced proliferation of smooth muscle cells from other tissues, such as vascular smooth muscle cells.

TABLE 4

| | A | B | C | D | E | F | G | H |
|---|---|---|---|---|---|---|---|---|
| | HASM-Thr | HASM- Thr | HASM- Thr | HASM - GM- | pFib - | ER-RLB | MCF7 | A549 |
| Compound ID | pIC₅₀ | pIC₂₅ | pIC₇₅ | CSF | bFGF | pIC₅₀ | pIC₅₀ | PIC₅₀ |
| 2-methoxyestradiol | 5.49 | 5.94 | 4.98 | 29 ± 10* | 62 ± 20* | 7.32 | 5.40 | 6.00 |
| CP-DM-2-11-7 | 6.90 | 8.82 | 5.66 | 45 ± 8* | 63 ± 15* | 5.82 | 4.00 | 4.89 |
| CP-DM-3-91 | 7.59 | 10.32 | 5.70 | 43 ± 11* | 49 ± 14* | 4.26 | 4.00 | 4.99 |
| CP-DM-3-106 | 7.60 | 9.35 | 6.36 | ND | ND | 6.75 | 4.00 | 5.12 |
| CP-DM-3-105 | 7.00 | 8.85 | 5.59 | ND | ND | 4.60 | 4.00 | 5.29 |
| CP-DM-3-124 | 7.35 | 9.35 | 6.36 | ND | ND | 6.05 | ND | ND |
| CP-DM-3-117 | 5.19 | | | ND | ND | 4.00 | ND | 5.29 |
| CP-DM-3-119 | 5.48 | | | 0 ± 0 | 17 ± 14 | 4.00 | 4.00 | 4.80 |
| CP-DM-3-118 | 5.53 | | | ND | ND | 4.68 | ND | ND |
| CP-DM-4-15 | 5.47 | | | 34 ± 11* | ND | 4.90 | 5.00 | ND |
| CP-DM-4-16 | 5.84 | 6.57 | 5.19 | ND | ND | 4.00 | ND | ND |
| CP-DM-4-36 | 6.89 | 7.91 | 5.69 | ND | ND | 4.70 | 4.00 | 5.00 |
| CP-DM-4-35 | 6.85 | 7.80 | 5.75 | 14 ± 24 | 22 ± 14 | 5.28 | 4.00 | 5.50 |
| CP-DM-4-37 | 4.89 | | | ND | ND | 5.19 | 4.00 | ND |
| CP-DM-4-51 | 5.36 | 5.46 | 5.26 | ND | ND | 4.46 | 4.00 | ND |
| CP-DM-4-52 | 6.02 | | | ND | ND | 6.40 | 4.00 | ND |
| CP-DM-4-38 | 4.00 | 4.00 | 4.00 | ND | 26 ± 26 | 4.00 | 4.00 | ND |
| CP-DM-3-101 | 5.72 | 6.27 | 5.06 | ND | ND | 5.62 | 4.90 | ND |
| CP-DM-3-102 | 7.05 | 8.03 | 5.99 | 14 ± 14 | 96 ± 4* | 5.62 | 4.00 | 4.90 |
| CP-DM-3-103 | 5.50 | | | ND | ND | 4.00 | 4.00 | 4.39 |
| CP-DM-3-104 | 6.26 | 7.16 | 5.02 | ND | ND | 4.00 | 5.07 | ND |
| CP-DM-4-5 | 6.87 | 10.46 | 5.27 | ND | ND | 6.10 | ND | 5.30 |
| CP-DM-4-6 | 6.93 | 9.57 | 4.94 | 28 ± 10* | ND | 4.34 | 5.64 | 5.11 |
| CP-DM-4-33 | 5.87 | 7.12 | 5.03 | ND | ND | 6.96 | 5.50 | 5.04 |
| CP-DM-4-34 | 4.00 | 4.00 | 4.00 | 29 ± 12 | ND | 4.39 | 5.39 | 5.67 |
| CP-DM-4-65 | 6.65 | | | 28 ± 31 | 32 ± 23 | 6.19 | 4.00 | 5.50 |
| CP-DM-4-66 | 5.00 | | | ND | ND | 4.00 | ND | ND |
| CP-DM-4-68 | 5.74 | 7.91 | 5.00 | 8 ± 13 | ND | 4.00 | ND | ND |
| CP-DM-4-62 | 5.66 | 5.69 | 5.63 | ND | ND | 6.00 | 4.00 | ND |
| CP-DM-4-63 | 6.46 | 7.70 | 5.62 | ND | ND | 6.87 | 4.00 | ND |
| CP-DM-4-64 | 5.61 | 6.56 | 4.81 | ND | ND | 5.80 | 4.00 | ND |
| CP-DM-4-67 | 5.00 | | | ND | ND | 4.00 | ND | ND |
| CP-DM-4-69 | 4.00 | 4.00 | 4.00 | ND | 87 ± 13* | 4.00 | ND | ND |
| CP-DM-4-74 | 5.94 | 6.59 | 5.34 | 11 ± 8 | 5 ± 9 | 4.00 | 5.00 | ND |
| CP-DM-4-75 | 5.59 | | | ND | ND | 4.00 | ND | ND |
| CP-DM-4-76 | 5.37 | | | ND | ND | 4.00 | ND | ND |
| CP-DM-4-77 | 5.52 | | | 26 ± 13 | ND | 4.00 | 4.00 | 5.50 |
| CP-DM-4-78 | 6.00 | | | 35 ± 9* | ND | 6.64 | 4.00 | 5.00 |
| CP-DM-4-88 | 4.80 | | | ND | ND | 4.00 | 4.00 | 4.00 |
| CP-DM-4-91 | 5.20 | | | ND | ND | 4.00 | 4.00 | 5.00 |
| CP-DM-4-89 | ND | ND | ND | ND | ND | 5.25 | ND | ND |

ND = not done
Compounds identified as having a pIC₅₀ of 4.00 were inactive

(b) Flow Cytometry Analysis of Cell Cycle Status

Cells seeded into 6-well plates at a density of 1.5×10⁴ cells/cm² and made quiescent as described previously were then stimulated with thrombin (0.3 U/ml) for 48 h. Monomed A was added to all cells (including control cells) at the time of mitogen addition.

Cells were detached from the culture plates by incubation with trypsin for 30 min at 37° C. (0.5% w/v). The resulting suspension was washed in PBS twice before resuspension in 1 ml of 70% ethanol for storage for up to 3 weeks at −20° C. Prior to staining, cells were washed twice (2% FCS in PBS) to remove the ethanol. Fixed cells were stained with propidium iodide (50 μg/ml) in Triton X-100 (0.1% v/v) with Rnase II (180 mU/ml). The cell suspension was passed through an 18-gauge needle to facilitate the separation of cell clumps. Cells were stored for 24 h before analysis.

Cell cycle status was analyzed using a Becton-Dickinson FACScan instrument (Becton Dickinson, NJ, USA). Ten thousand events from each sample were counted and analyzed using a ModFitLT V2.0 analysis package (Verity Software House, ME, USA).

There was no evidence of cytotoxicity at concentrations below 3 μM as evidenced by lack of cell detachment from the culture plate and lack of staining of cells with trypan blue (fewer than 5%). The FACs profiles suggest that apoptosis is not a feature of the action of the analogues, since there was no accumulation of sub G0/G1 DNA content. Thus, these analogues appear to provide a specific anti-proliferative effect on airway smooth muscle cells. Without wishing to be bound to any particular proposed mode of action, these results suggest that the compounds of the application act via the modulation of intracellular signalling pathways.

The effects of 2MEO and 4NOM on the shape of HASM in the presence thrombin (0.3 U/mL) was investigated. Analogues were pre-incubated with HASM for 30 min before the addition of thrombin. The incubation continued for 48 h before capturing images of the HASM.

Importantly, at concentrations equi-effective for inhibition of proliferation, 4NO (1 μM) had no detectable effects on the shape of the HASM cells investigated, whereas 2MEO (10 μM) caused extensive rounding of cells.

The effect of other compounds on the cell cycle and cell death may be investigated using the same assay system.

### Example 42

### Effects of Test Compounds on Proliferation of Non-Smooth Muscle Cells

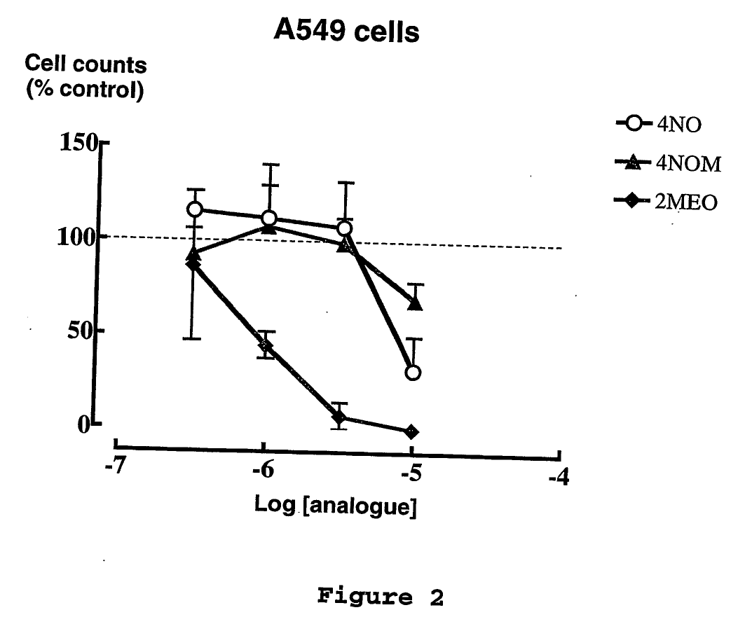

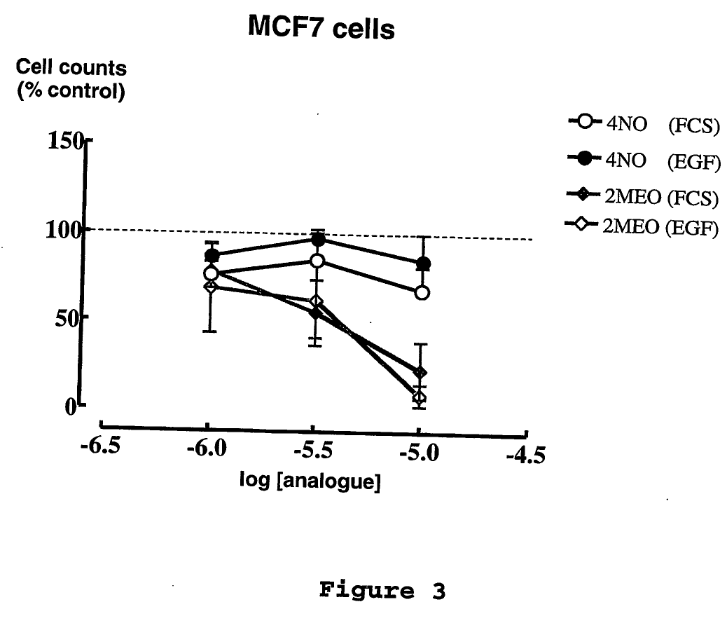

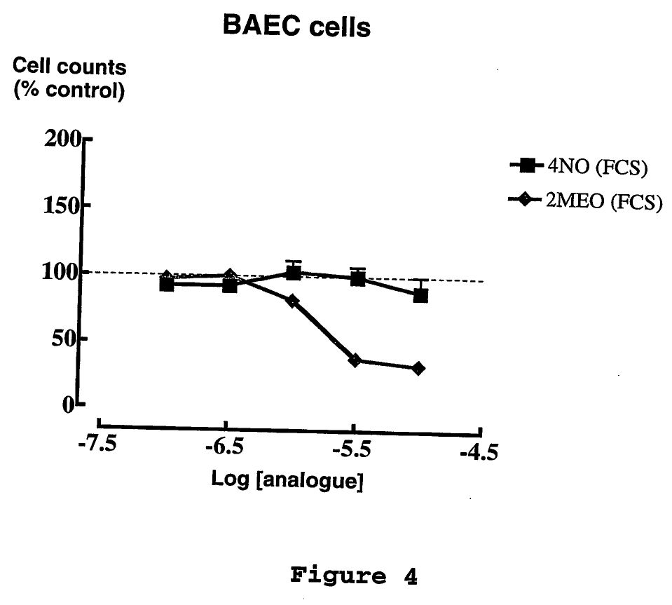

The effects of 4NO were evaluated in a number of non-smooth muscle cell types including the type II airway epithelial cell line, A549 (ATCC accession number ATCC CCL-185 Lung carcinoma human) (FIG. 2); the human breast tumour cell line, MCF7, (ATTC accession number ATCC HTB-22 breast adenocarcinoma, human) which expresses the estrogen receptor (FIG. 3); and in bovine aortic endothelial cells (BAEC) (FIG. 4).

The effects of test compounds were examined on proliferation of A549 cells in response to 5% fetal calf serum (FCS). Analogues were pre-incubated with A549 cells for 30 min before the addition of FCS. The incubation continued for 48 h before cell enumeration. Data are expressed as a percentage of the cell number in vehicle treated wells and are presented as the mean and standard error of the mean. Similarly the effects of 2MEO and 4NO on proliferation of MCF7 in response to 5% FCS or epidermal growth factor (EGF, 300 pM) were examined, as were the effects of 2MEO and 4NO on proliferation of BAEC in response to 5% FCS. The incubation parameters were the same as described above and results are presented as the mean and standard error of the mean.

Surprisingly, at concentrations below 3 μM, 4NO had no detectable effect on the proliferation of any of these cell types, suggesting specificity of this compound towards smooth muscle cells and fibroblasts and not to a variety of other cells also found in the airway. In contrast, 2MEO was an effective inhibitor of the proliferation of each cell type.

This assay was also used to evaluate the ability of other compounds to inhibit the proliferation of non-smooth muscle cells MCF7 and A549 (described above) in response to 5% FCS. The results of this assay are presented in Table 4 (columns G and H respectively). The potency of the compounds was considerably less in inhibiting proliferation of these cell lines than it was for HASM. These observations support the contention that there are cell type-specific targets for the actions of these compounds. Such assays may also be adopted to examine the effect of other disclosed compounds on these cells or on other non-smooth muscle cells of interest.

### Example 43

### Comparison of Test Compounds with Dexamethasone on Inhibition of HASM Proliferation

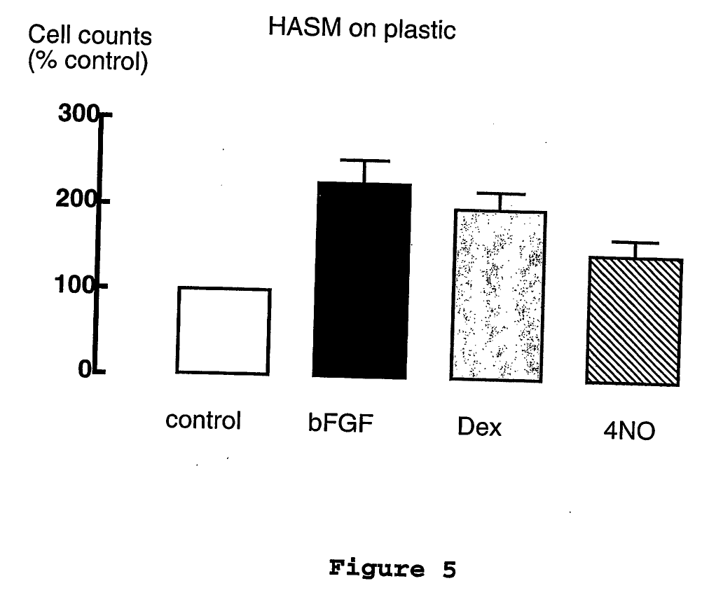

We examined the effects of 4NO (1 μM) and the glucocorticoid dexamethasone (Dex, 100 nM) on the proliferation of HASM cells in response to bFGF (300 pM). Dexamethasone is a commonly used anti-inflammatory glucocorticoid. Cells were cultured on a standard plastic tissue culture plate. The test compounds were pre-incubated with HASM for 30 minutes before the addition of bFGF. The incubation continued for 7 days before cell enumeration (FIG. 5). Data are expressed as a percentage of the cell number in vehicle treated wells and are presented as the mean and standard error of the mean.

The proliferation of HASM cells on plastic culture plates was attenuated, but not completely blocked by dexamethasone. In contrast, advantageously 4NO markedly reduced the increase in HASM cell number to levels below that of dexamethasone.

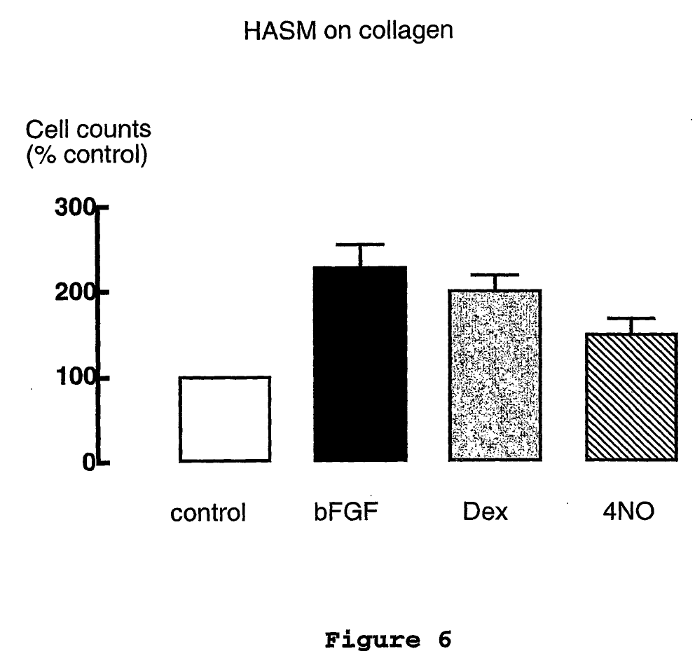

The response of HASM cells grown on a collagen extracellular matrix to the glucocorticoids and test compounds was also examined. Cells were cultured on a collagen-coated silastic base. HASM cells were pre-incubated with the test compounds for 30 minutes before the addition of bFGF (300 pM). The incubation continued for 7 days before cell enumeration (FIG. 6). Data are expressed as a percentage of the cell number in vehicle treated wells and are presented as the mean and standard error of the mean.

Surprisingly, the proliferation of HASM cells on the collagen ECM was resistant to regulation by dexamethasone, suggesting the attenuation of the effect of this compound in the presence of collagen-containing matrices. 4NO, however, retained its effectiveness as an anti-proliferative agent for. cells on a collagen ECM. These observations indicate that HASM cell proliferation is better controlled by the presently, disclosed compounds than by glucocorticoids.

### Example 44

### Affinity of Test Compounds for the Estrogen Receptor and Tubulin

Estrogen Receptor Binding Assay

The Estrogen Receptor (ER) affinity of estradiol (E2), 2MEO and analogues were determined using rat uterine cytosol as a source of ER (Markaverich et al., 1979) as described in detail (Hughes et al., 2002). Uteri (6-10 per batch) were isolated from Brown Norway rats (250-350g), washed free from blood in saline and homogenised in 10 mM Tris buffer (pH 7.4, 1.5 mM EDTA, 10% w/v glycerol, 1 mM phenylmethylsulfonylfluoride) using an Ultra Turrax homogeniser for 3 by 15 s bursts with intervals of 1 min on ice.

Nuclear material and cellular debris were removed by centrifugation at 750×g at 4° C. for 10 min (Sorvall RT7); the cytosol component was prepared by centrifugation at 30000×g for 120 min at 4° C. in a Beckman JM1 centrifuge. The cytosol was diluted to approximately 1 mg/ml protein (rat uterine extract) determined using the Bradford method (Biorad).

Binding assays were carried out by overnight incubation at 4° C. in 300 μL of the above-described buffer, comprising 200 μL of cytosol, 50 μL of displacer (10 μM estradiol or buffer) and 50 μL of 0.2 nM [³H]-E2 (150 Ci/mmol). Separation of bound from free radioligand was achieved by the addition of 500 μL of dextran-coated charcoal (400 mg dextran clinical grade C and 2g of charcoal, Norit A in 100 mL of Tris buffer) and centrifugation at 4° C. in a Sorval RT7 at 2000×g for 10 min.

Saturation analysis over the range 0.01-50 nM [³H]-estradiol yielded a K_{d} of 0.18 nM and Bmax 112 fmol/mg protein (n=3, analysis using Graph Pad Prism™), indicating that the 0.2 nM concentration was suitable for displacement studies. Analyses of 2MEO and analogues were carried out over the range 0.1 n -10 μM in 0.5 to 1.0 log increments and following identification of the appropriate range, displacement of estradiol was determined using 3-4 concentrations per decade.

The data were fitted to the Cheng and Prussof equation using the K_{d} of 0.18 nM (as determined by saturation analysis) and the data are presented as pIC₅₀ (negative logarithm of the concentration producing 50% of the maximum displacement of radiolabel).

The results are presented in Table 5.

Purification of Tubulin for Colchicine Binding Assays

The ability of 2MEO and its analogues to displace ³H-colchicine from its binding sites on partially purified tubulin was examined. For the binding assays, a partial purification of tubulin was derived from the polymerization-depolymerization method of Williams and Lee (Williams et al., 1982). Three lamb brains (approximately 150 g total) were obtained approximately fifteen minutes after slaughter. Batches of 100g of brain were homogenized in 50 mL cold PM4M buffer (100 mM piperazine-N,N′-bis[2-ethane sulfonic acid] (PIPES), 1 mM MgSO₄, 2 mM ethylene glycol tetra-acetate (EGTA), 2 mM DTT, 4 M glycerol, pH 6.9) using a domestic blender (Power Blender, Ronson) on the highest setting for 30 s. The crude homogenate was centrifuged for 15 mins (9000 rpm (6500×g), 4° C.) and the precipitate (cell debris, extracellular matrix and crude nuclear fraction) discarded. The 6500×g supernatant was then centrifuged for 75 mins (28 000 rpm (96 000×g), 4° C.) and the precipitate (consisting largely of cell membranes) was discarded.

The GTP (guanosine triphosphate) concentration of the 96 000×g supernatant was adjusted to 1 mM by the addition of an appropriate amount of a freshly prepared solution of 50 mM GTP in distilled water. Polymerization of the tubulin was accomplished by heating in a water bath at 37° C. for 45 mins. The crude tubulin was then pelleted by centrifugation for 60 mins (28 000 rpm (96 000×g), 27° C.). The tubulin-rich precipitate was then gently resuspended in 15 mL buffer (1 M monosodium glutamate, 1 mM MgCl₂, pH 6.6) using a hand-held tissue homogenizer. The partially purified tubulin was then incubated on ice for 30-60 minutes and frozen at −80° C.

According to Williams and Lee (1982), the 30-60 minute incubation on ice should depolymerize the tubulin (Williams t al., 1982). However, fractions that were clarified for 60 in (28 000 rpm, 4° C., 60 minutes) following this incubation showed no specific binding of colchicine, suggesting that the tubulin was still polymerized and in suspension. Therefore, the fraction produced after the 30-60 min ice incubation was used without further purification.

Colchicine Displacement Assay

Determination of affinity for the colchicine-binding site of tubulin was accomplished by displacement of ³H colchicine. Reaction components were derived from the method of D'Amato and colleagues (D'Amato et al., 1994), while the separation of bound from free radioligand on 1 ml size exclusion columns was adapted from the method of Penefsky (Penefsky, 1977). These micro-columns were successfully used by Hamel and colleagues (Hamel et al., 1996) to study the characteristics of ³H 2-methoxyestradiol binding to tubulin. Each micro-column consisted of a column of Sephadex G-50 (fine grade) in a 1 mL disposable syringe filled to approximately 0.95 ml. Duplicate reaction mixtures (total volume 0.48 ml) contained approximately 0.5 mg/mL partially purified tubulin, 1 μM ³H colchicine, and displacer compound in buffer (1 M monosodium glutamate, 1 mM MgCl₂, 0.5 mg/mL bovine serum albumin (BSA), pH 6.6). Saturation studies indicated a K_{D} for colchicine in this assay of 1.4 μM, implying 1 μM was an appropriate radioligand concentration. Half-decade increments of displacer were used (range 10^{-7.5} to 10⁻⁴ M).

Reaction mixtures were incubated for 30 mins at 37° C. in a water bath, followed by cold stabilization on ice for 1 to 1.5 hours. For each reaction mixture, three aliquots of 0.14 ml were then added to separate, pre-cooled micro-columns. Separation was accomplished by centrifugation for 2 mins (1100 rpm (100×g), 4° C.). 5 ml “Emulsifier Safe” scintillation fluid (Packard) was added to each tube, and the samples counted in a Packard 1600TR liquid scintillation analyzer. The data were fitted to the Cheng and Prusoff equation and both Kᵢ and pIC₅₀ values calculated (GraphPad Prism).

TABLE 5
Affinities of 2MEO and analogues for estrogen receptors
and the colchicine binding site on tubulin.

| | | Tubulin affinity |
|---|---|---|
| | ER affinity (pIC₅₀ | (pIC₅₀ ³H-colchicine |
| Analogue | ³H-E2 displacement) | displacement) |
| E2 | 9.5 ± 0.1 | <4.0 |
| 2MEO | 6.8 ± 0.1 | 4.8 |
| 4NO | <5.0 | <4.0 |
| 4NOM | <5.0 | NA |

NA = not available

2MEO had a pIC₅₀ value of 6.8 (Table 5), whereas the 2MEO analogues, 4NO and 4NOM had little or no detectable displacement of ³H-Estradiol at concentrations up to 10 μM. Neither 4NO nor 4NOM displaced 50% of the E2 binding at this concentration and therefore their affinity constants could not be determined (Table 5).

The results from the colchicine displacement experiments demonstrated that 2MEO had a pIC₅₀ of 4.8 for tubulin, whereas none of the test compounds showed significant displacement of colchicine in concentrations of up to 100 μM. Given the relatively weak affinity of the test compounds for tubulin these compounds the anti-proliferative activity of the test compounds appears unlikely to be mediated by the general disruption of microtubule dynamics.

A number of the disclosed compounds were subsequently examined for their ability to displace 3H-estradiol from cytosolic preparations of rat uterus as described above. The results of these assays are summarized in Table 4, Column F. Without exception, the compounds exemplified in Table 4 had less affinity for the cytosolic estrogen receptor than did 2-methoxyestradiol.

Given the very low affinity of the test compounds for ER, it would appear that the mode of action of these compounds is not via this receptor.

### Example 45

### Effects of Test Compounds on Interleukin-1α-Mediated GM-CSF Production by Human Airway Smooth Muscle Cells

Granulocyte-macrophage colony-stimulating factor (GM-CSF) is a cytokine produced by a wide variety of different cell types under stimulation by pro-inflammatory stimuli as diverse as interleukin-1, lipopolysaccharide and diesel exhaust (Ritz et al., 2002). GM-CSF is implicated in a range of different inflammatory conditions including rheumatoid arthritis, asthma, sepsis and allergic rhinitis. It is likely that GM-CSF is involved in all tissue inflammation to varying degrees (Hamilton, 2002).

It has now been established that mesenchymal cells, fibroblasts and smooth muscle cells, make a significant contribution to the burden of cytokines in inflammatory disease. In particular there is abundant evidence of the capacity of airway smooth muscle cells to produce inflammatory cytokines (Panettieri, 2002). These links enable the present suggestion that the inhibitory effect of 4NO on GM-CSF would constitute a potential important anti-inflammatory effect. To explore the potential anti-inflammatory activity of 4NO, its effect on interleukin-1α(1 ng/ml)-mediated HASM production of granulocyte-macrophage colony-stimulating factor (GM-CSF) was examined.

Measurement of GM-CSF Levels

Levels of GM-CSF were quantified using ELISA. ELISA plates were coated with rat anti-human GM-CSF monoclonal coating antibody (1 μg/ml, Endogen, MA, USA; in 0.1 M sodium carbonate buffer, pH 9.4-9.8) and then left to incubate overnight at room temperature.

The plate was washed three times with buffer (PBS containing 0.1% Tween-20, PBS-T) before incubation with a blocking buffer (PBS containing 4% FCS) for 1 h. Biotin-labelled, rat anti-human GM-CSF antibody (0.25 μg/ml, Endogen) diluted in PBS was incubated with samples or human recombinant GM-CSF standards (ranging from 0-1000 pg/ml, Endogen) for 2 h at room temperature before extensive washing. The signal was generated during a 30 min incubation with poly HRP-streptavidin solution (Endogen) and the substrate, 3,3′,5,5′-Tetramethylbenzidine (BD PharMingen, San Diego, Calif.) according to methods supplied by the manufacturer (Endogen) and absorbance was determined at 450 nm (Victor 1420 Multilabel Counter, Wallac).

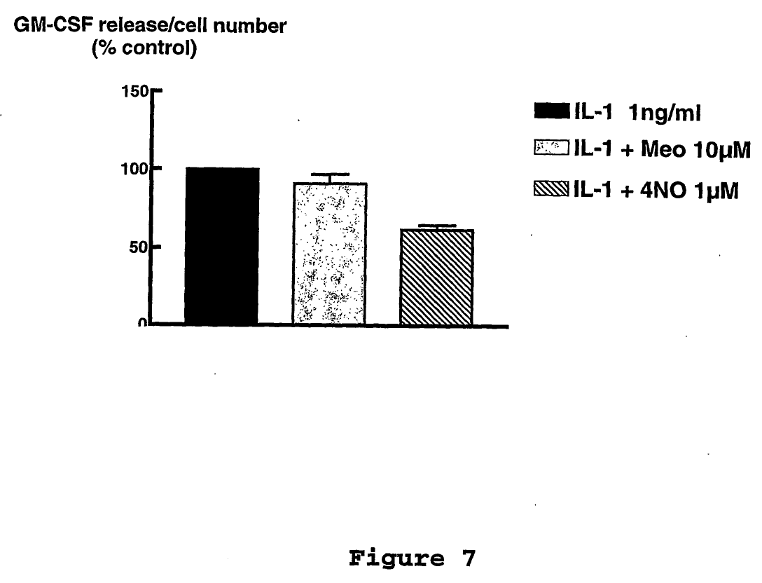

The results of these experiments are presented in FIG. 7. Data are expressed as a percentage of the GM-CSF level detected in the culture supernatant of the test compound pretreated IL-1α-stimulated cells and are presented as the mean and standard error of the mean. 4NO reduced GM-CSF production to greater extent than 2MEO.

Other disclosed compounds were subsequently tested for their ability to inhibit GM-CSF production in the assay system described above. These results are summarized in Column D of Table 4. While three of the compounds tested, CP-DM-3-119, CP-DM-3-103 and CP-DM-4-35 either did not inhibit or produced less inhibition of GM-CSF production by HASM cells when compared with 2-methoxyestradiol, one compound, CP-DM-3-102 was dramatically more effective. The reasons for this variation in inhibitory activity are presently not clear and the number of samples tested at each point was relatively small; further experiments to increase the number of samples to be tested may assist in confirming these results. Other disclosed compounds could also be tested using this assay system.

A reduction in GM-CSF production in smooth muscle could have an important influence on the intensity and duration of an inflammatory response. Moreover, it is likely that the inhibitory effect of 4NO on GM-CSF production will occur in many of the other cell types that produce GM-CSF since other agents, such as glucocorticoids, appear to act on all cell types that make GM-CSF to inhibit its production

### Example 46

### Effects of Test Compounds in Animal Model of Airway Hyperresponsiveness

The mouse has been used extensively to characterize the pathological basis of allergic responses within the airway and to investigate the action of existing and novel anti-asthma agents. The mouse model is regarded by many investigators as a critical model in which to obtain evidence of potential efficacy of novel anti-asthma agents (Gleich & Kita, 1997) and it is widely used for this purpose in the pharmaceutical industry. This application is supported by the mouse model showing the efficacy of the most important class of preventer drugs, the glucocorticoids (see for example, Walter et al., 2002).

Sensitisation and Antigen-Challenge

Conscious female mice (C57BL/6) of 6-8 weeks of age were sensitised by a 0.2 ml intraperitoneal injection of 50 ug ovalbumin (in 10 mg/ml aluminium hydroxide) on day 0 and 12. and subsequently challenged with an aerosol of OVA (1% w/v)/FCS (5% v/v) on days 20-28 for 30 minutes. The aerosol exposures were achieved by placing the rodents in a whole body plethysmograph apparatus and then nebulising the drug into the chamber through a port located on the top of the chamber. The aerosol exited the chamber via a bias flow into a particle trap. The antigen-challenge did not cause a detectable increase in airways resistance and there are no signs of distress.

Drug treatments were administered to assess the effects of drugs on the development of airway hyperresponsiveness. Drug treatment commenced the day prior (day 19) to the first exposure to OVA (day 20) and continued throughout the period during which the mice were challenged with OVA. Drug treatment comprised 4NO or 2MEO, each at 50 mg/kg administered once daily 2h before OVA exposure to either intraperitoneally or orally in a vehicle of 10% DMSO/90% peanut oil in a volume of 100 μL.

After the 26-28 day sensitisation and challenge protocol, airway obstruction in response to the bronchoconstrictor methacholine was measured. The mice were anaesthetised with a mixture of ketamine/xylazine. A tracheostomy was performed, and a catheter placed in the jugular vein to administer methacholine intravenously. The mouse was then be placed in a whole body plethysmograph and mechanically ventilated (150 breaths/min at a tidal volume of 10 ml/kg). The transpulmonary pressure was measured with a differential pressure transducer with one port attached to the interior of the plethysmograph and the other to the intratracheal cannula. The airflow rate was measured with a pneumotachograph.

The change in airways resistance in response to intravenous methacholine (1-1000 μg/kg, given 4 minutes apart) was calculated on-line with a Buxco pulmonary mechanics analyzer.

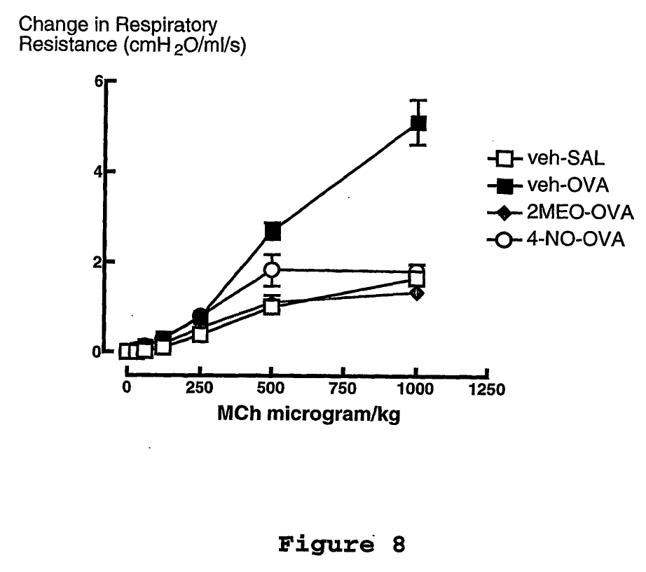

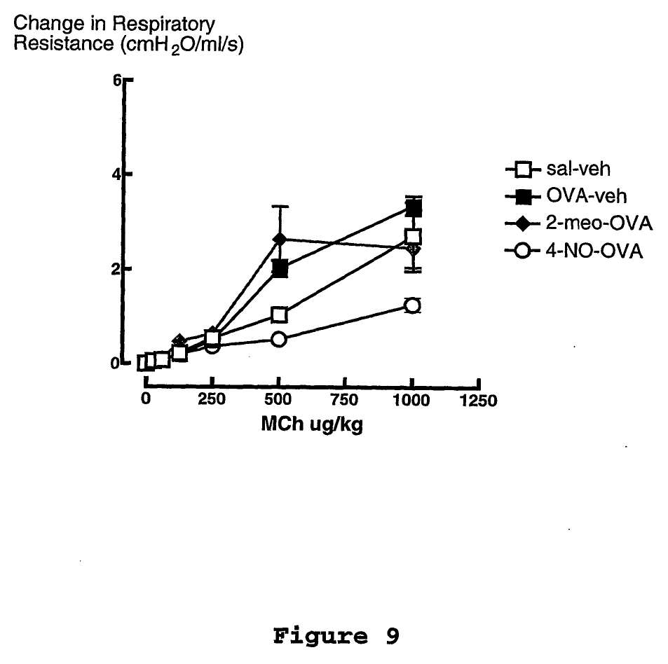

The results of these experiments are shown in FIGS. 8 and 9.

The effects of treatment of ovalbumin-challenged mice (veh-OVA) with either 2MEO or 4NO, provided intraperitoneally at 50 mg/kg/day on airway reactivity to intravenously administered methacholine are shown in FIG. 8. The effects of treatment with the test compounds provided orally at 50 mg/kg/day are presented in FIG. 9. Data are expressed as the change in respiratory resistance from the baseline level and are presented as the mean and standard error of the mean.

OVA challenge (veh-OVA) increased the airway reactivity to methacholine compared to that detected in non-challenged mice (veh-SAL).

Both 4NO or 2MEO, when administered either intraperitonally or orally, were able to reduce the increase in respiratory resistance which was exhibited following challenge.

### Example 47

### Effects of Test Compounds on Human Pulmonary Fibroblast Proliferation

Samples of parenchyma from human lung transplant recipients were dissected away from the pleura, chopped into fragments of less than 1 mm² and allowed to adhere to plastic culture dishes in a minimal volume of DMEM containing serum (10%) and antibiotics (as used for HASM culture). After the tissue fragments had adhered to the base of the dish additional medium was added. Medium was changed twice weekly until an explant culture formed, at which time the tissue fragments were removed and the cells were passaged in a 1:3 split ratio by exposure to trypsin weekly to produce pulmonary fibroblast cultures.

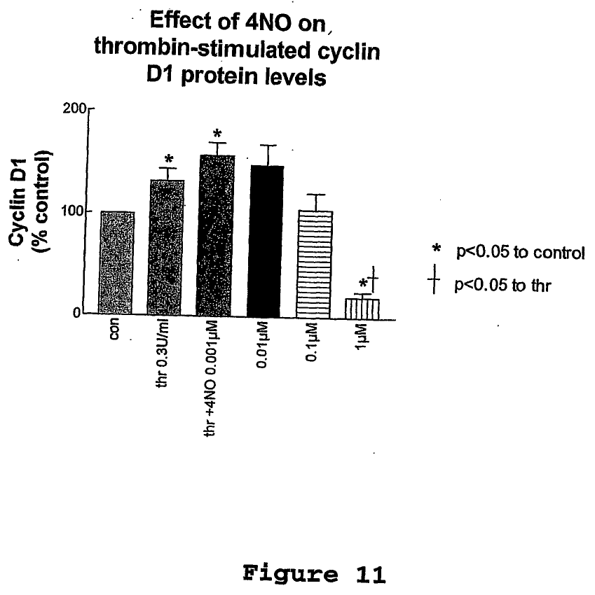

Pulmonary fibroblasts were cultured in DMEM, serum-deprived for 24 h, in the presence or absence of the test compounds, and then stimulated with 300 pM bFGF for 48 h before harvest and counting of viable cells. The results of these experiments are shown in FIG. 11. An increase pulmonary fibroblast cell number was prevented by 4NOM over the concentration range of 1 to 1000 nM. 4NO significantly reduced the proliferative response of pulmonary fibroblasts to bFGF over the same concentration range.

A range of the disclosed compounds was subsequently examined using the above assay system, and the results are summarized in Column E of Table 4. Data are presented as the mean and standard error of the mean of the inhibition of bFGF (300 pM)-induced proliferation in a minimum of 3 separate experiments in cell lines from at least 3 donors. At 1 μM, 4NOM and several other compounds showed significant inhibitory activity. The proliferation of parenchymal fibroblasts is important in the development of pulmonary fibrosis. Thus, the current observations suggest that the disclosed compounds have therapeutic potential in the treatment of fibrotic conditions in the airway and lungs.

Airway fibroblasts were also obtained by explant culture from airway biopsies. A more limited set of experiments revealed that at 3 μM 4NO and 4NOM reduced bFGF (300 pM) induced proliferation by 55±20% and 50±24%, respectively. Thus, it appears that these compounds have activity against fibroblasts from diverse tissue sources suggesting therapeutic potential in conditions related to fibroblast dysfunction. This is further supported by inhibitory effects of 4NO and 4NOM of 26±12% and 68±10%, respectively, on thrombin-induced proliferation of NIH3T3 cells, which are of fibroblast origin, suggesting that the compounds may reasonably be predicted to have anti-fibrotic activity in other sites of the body.

These assays may also be used to determine the anti-proliferative activity of other disclosed compounds to fibroblasts from these and other tissue sources.

### Example 48

### Effect of Test Compounds on Cyclin D1 Expression

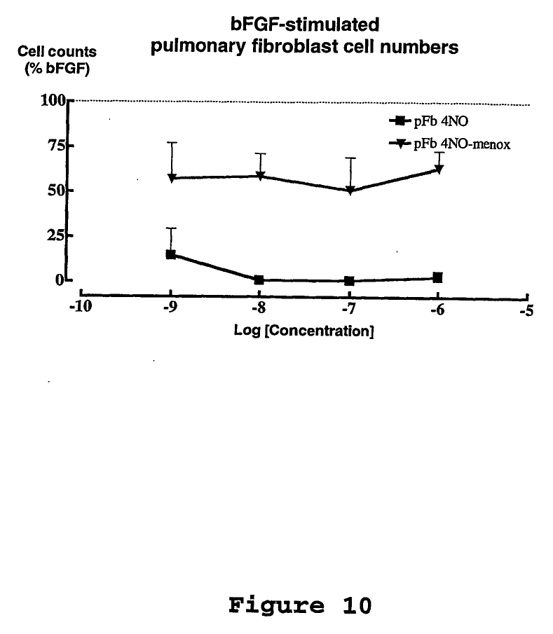

Human airway smooth muscle cells were cultured as described above in the proliferation experiments. Cells were serum-deprived for 24 h and stimulated with thrombin (0.3 U/ml) for 8h before harvest of protein for Western Blotting of cyclin D1. Pre-incubation of HASM with 4NO (0.01 μM-1 μM) produced a concentration-dependent reduction in the cyclin D1 protein levels. These results are illustrated in FIG. 10. The reduction in cyclin D1 suggests that cell entry into S-phase will be delayed and/or reduced and it therefore may explain part of the reduction in cell number observed at the 48h time points in the proliferation assays.

### Example 49

### Effect of Test Compounds on Human Airway Smooth Muscle Cell Migration

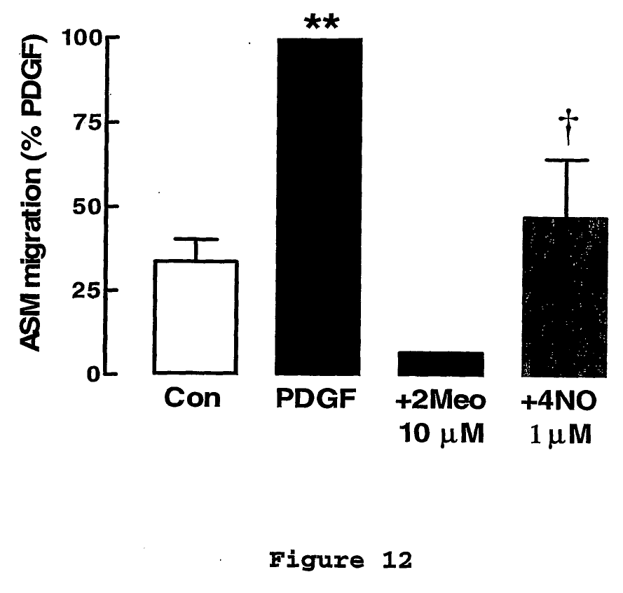

Costar transwell culture inserts (Corning Inc, USA) with 6.5 mm diameter and 8 mm pore size were coated overnight at 4° C. with 0.1% gelatin (Bovine skin; Sigma, USA) prior to the commencement of the assay (modified Boyden chamber assay). HASM cells were deprived of serum for 24 h prior to the commencement of the assay. Cells were added to the upper side of the transwell inserts with or without 30 min pre-treatment with one of the test compounds. Platelet-derived growth factor(BB) (1 ng/ml) was added to the lower compartment to stimulate cell migration and the chambers and cells left for 5 h. After this period the membranes with the adherent cells were washed with PBS, and fixed for 2 min with DiffQuick (Lab Aids, Aus) fixative and then stained with DiffQuick. Membranes were then washed twice in PBS, peeled off the inserts and placed onto slides. Membranes were visualised by light microscopy, and the number of migrated cells in five fields (×400) were counted in triplicate. The results of this experiment are presented in FIG. 12.

The numbers of HASM appearing on the underside of the porous membrane was counted and the responses were expressed as a percentage of the number of cells migrating in response to PDGF in the absence of pre-treatment. HASM pretreated with either 2MEO or 4NO showed significantly reduced levels of migration in response to PDGF.

### Example 50

### In Vitro Assessment of Smooth Muscle and Fibroblast Related Pathology

Vascular Smooth Muscle

The evaluation of effects of drugs on proliferation of vascular smooth muscle is a well-established screen for activity of drugs that have utility in the prevention of vascular remodelling. Vascular remodelling occurs in systemic and pulmonary hypertension, atherosclerosis and restenosis following arterial (coronary etc) angioplasty.

Vascular Smooth Muscle Proliferation

Vascular smooth muscle is cultured according to methods described previously described in detail (Campbell and Campbell 1993). Vascular smooth muscle is obtained from either rat aorta, bovine aorta or from the bovine pulmonary artery. The endothelium is removed by abrasion and the media is dissected away from the adventitia. The media is then chopped into small (2 mm³) fragments and digested in collagenase and elastase until a suspension of single or few cells is generated. The cell suspension is then washed twice in phosphate buffered saline (PBS) (containing 2% fetal calf serum) (FCS) and the cells are seeded into one 75 cm² flask in DMEM containing antimicrobial agents and 10% fetal calf serum. Cells are passaged weekly by exposure to trypsin (0.5% w/v in Ca²⁺-free PBS containing 0.1% w/v EDTA) (Tomlinson, Croft et al. 1994).

VASM proliferation is assessed by seeding cells at a sub-confluent density of 10⁴ cells/cm² into 6 well tissue culture plates. After 72 hours the medium is exchanged for medium deficient in fetal calf serum. After a further 24 hours the cells are stimulated with a range of concentrations of specific mitogens together with a serum-free medium supplement containing insulin, selenium and transferrin. When compounds of the present application are to be evaluated they are added 30 min prior to the mitogens at a range of concentrations between 1 p M and 10 μM. Following a 48 hour incubation of the cells with the mitogen, the cells are removed from the tissue culture plate by exposure to trypsin. The cell suspension is isolated by centrifugation at 1000 g (ambient temperature) and the cells are re-suspended in PBS containing 2% FCS prior to counting using a haemocytometer. In addition an aliquot of cells is incubated with propidium iodide in the presence and absence of a permeabilising fixative buffer containing RNase to enable the enumeration of non-viable cells and to prepare cells for flow cytometric assessment of DNA content as described in previous studies (Berk, Elder et al. 1990; Fernandes, Guida et al. 1999).

Previous studies evaluating regulation of smooth muscle proliferation in vitro in cultured cells have shown anti-proliferative effects of agents that are useful in treating smooth muscle related conditions such as atherosclerosis (Hupfeld and Weiss 2001; Mattingly, Gibbs et al. 2002), and so these assays may be used in predicting the efficacy of compounds of the disclosure.

Smooth Muscle Hypertrophy

The hypertrophic response of smooth muscle is important in a number of conditions including hypertension, airway wall remodelling and prostatic enlargement. Hypertrophic responses of cultured smooth muscle can be assessed by measuring the amount forward light scatter in unfixed cell suspensions (Uhal, Ramos et al. 1998)). Cells are seeded into 6 well tissue culture plates and after 48-72 hours the culture medium is replaced with one deficient in fetal calf serum for a period of 24 hours. Potential hypertrophic factors are added together with a mixture of insulin, transferrin and selenium for variable periods of up to 7 days at which time adherent cells are harvested by brief exposure to trypsin. The cells are incubated in PBS containing 2% fetal calf serum and propidium iodide to enable exclusion of non-viable cells from the analysis. Increases in forward light scatter indicate an increase in cell size. In addition, hypertrophy can be measured indirectly by measuring the amount of protein per cell (Isaeff, Goya et al. 1993; Blennerhassett, Bovell et al. 1999) or by measuring the protein synthesis rate normalised to DNA content of the culture (McKay, de Jongste et al. 1998).

Smooth Muscle Contractile Function

Preparations of vascular, airway, uterine, bladder & gastrointestinal smooth muscle are mounted in a glass-jacketed organ bath maintained at 37° C. in a standard physiological salt solution (e.g. Krebs solution). The tissue is prepared as a ring or a strip which is then suspended between metal hooks, one end being fixed to a clamp, the other end being attached to a previously calibrated force transducer. The influence of the 2MEO analogues is investigated by their direct addition to the organ bath in increasing concentrations between 1 pM and 10 10 μM. Indirect effects are assessed by examining the influence of the analogues on either the contractile or the relaxant responses of the preparation to stimuli that are relevant for that particular tissue. These methods are in common usage and are exemplified in the following citations (Armour, Lazar et al. 1984; Arthur, Yin et al. 1997; Andersen, Weis et al. 1999)

Smooth Muscle Cytokine Production

It has been evident that smooth muscle of diverse tissue origin has the capacity to produce a large range of different cytokines when subject to appropriate stimulation(John, Au et al. 1998). Cytokine production is readily measured by harvesting the supernatant of cells maintained in culture and incubated with 2MEO analogues in addition to the stimuli for cytokine production which include lipopolysaccharide, interleukin-1α, tumour necrosis factor-α inter alia. Cytokines elaborated by smooth muscle include granulocyte-macrophage colony-stimulating factor, interleukin-8, eotaxin and RANTES. These cytokines are measured by enzyme-linked immunosorbent assay (ELISA) to determine secreted protein levels. In addition, specific messenger RNA for each cytokine can be measured quantitatively by real-time PCR or by Northern blotting.

Fibroblast Function

A number of fibroblast functions, measurable in cultured fibroblasts of different tissue origin, are regarded as predictive of activity in conditions in which there is a fibroblast contribution to pathogenesis. Such conditions include but are not limited to pulmonary fibrosis, airway fibrosis including bronchiolitis obliterans, cardiac fibrosis and fibrosis occurring in other tissues. These functions include proliferation, cytokine production, extracellular matrix production, cell migration and contraction and are readily measured in cell cultures of fibroblasts derived from the organ relevant to the pathology (Bishop, Mitchell et al. 1993; Butt, Laurent et al. 1995; Dube, Chakir et al. 1998).

Fibroblast Culture

Fibroblasts may be cultured from a wide range of tissue biopsies including skin, lung parenchyma, airway biopsy, cardiac tissue and foreskin. Fibroblasts from airway biopsy are grown by explant culture Dube, Chakir et al. 1998. In addition, as fibroblast growth characteristics may be altered by disease processes fibroblasts are also obtained from biopsy tissues of fibrotic organs such as the peripheral lung (Ramos, Montano et al. 2001).

### Example 51

### Preclinical Animal Models in Assessment of Smooth Muscle and Fibroblast Related Pathology

Atherosclerosis/Restenosis

A number of animal models of atherosclerosis/restenosis are commonly used for assessing the impact of drugs on the development of an intimal lesion (Karas 2002 (Hodgin and Maeda 2002). The merits of such models have been discussed in recent publications (Van Put, Van Hove et al. 1995; Hickey, Makdissi et al. 1996). Murine models showing varying degrees of ApoE deficiency demonstrate development of atherosclerotic lesions that can be accelerated by dietary modification. In addition, models in the rabbit using a non-occlusive cuff around the carotid arteries induce neo-intimal lesions within a 7-10 day period with minimal direct endothelial injury. These models of atherosclerosis are routinely used to screen for novel anti-atherosclerotic agents and show that a number of compounds that are active in human disease are also active in the models (Arthur, Yin et al. 1997; Karas 2002). In this model, endothelial dysfunction is detectable prior to the development of the intimal lesion. Plaque rupture can be induced in a variety of complex models of atherosclerosis and has also been reported in the ApoE deficient murine model (Rekhter 2002)). Restenosis can be more specifically modelled by balloon catheter denudation on the rat carotid artery endothelium (Nili, Zhang et al. 2002).

Prostatic Hyperplasia

Prostatic hyperplasia is investigated in rats or mice treated chronically with subcutaneous injection of a sympathomimetic agent such as phenylephrine (Marinese et al., 2003)

Pulmonary Hypertension

The development of pulmonary hypertension in experimental animals can be induced by chronic exposure to hypoxia or by injection of monocrotaline (Jeffery and Wanstall 2001; Wanstall, Gambino et al. 2002). Hypoxia in mice or rats causes right ventricular hypertrophy and increases in pulmonary artery thickness that can be detected by histological methods following housing of animals in an environment containing 10% oxygen for a period of 4 weeks.

Systemic Hypertension

There are vast numbers of models in which to evaluate the anti-hypertensive activity of the presently claimed compounds. Reference to electronic databases such as PubMed will enable a skilled investigator to select a variety of models from different species. It would be appreciated that the agents are evaluated in models that have a genetic component (eg in the spontaneously hypertensive rat, SHR), in models that have a significant renal component and in models that have concurrent obesity and/or diabetes(see for example, (Haff, Page et al. 1977; Dominiczak, Devlin et al. 1997; Sechi 1999; Gerin, Pickering et al. 2000; Stoll and Jacob 2001; Sugiyama, Yagami et al. 2001). Moreover, in addition to measuring impact on systemic blood pressure, associated pathologies such as left ventricular hypertrophy (determined as the ratio of the left ventricular:total body weight ratio) are measured.

Fibrotic Diseases

Although there are many models of fibrotic diseases in experimental animals, bleomycin-induction appears to be the single most common method of disease induction. For example, local subcutaneous injection in mice over a period of 14 days induces sclerosis of the skin with readily measurable increases in dermal thickness and skin collagen content (Murota, Hamasaki et al. 2003). Of particular relevance to the evaluation of the disclosed compounds is the well-characterised model of pulmonary fibrosis induced by intra-tracheal injection of bleomycin (Chang, Nakao et al. 1980; Evans, McAnulty et al. 1990; Tani, Yasuoka et al. 1991).

The activity of CP-DM-2-11-7 was investigated using a bleomycin model of pulmonary fibrosis (Underwood et al. 2000). 4NO was administered daily commencing on the day of the bleomycin challenge, 2 hours before the mice received bleomycin. The 4NO (50 mg/kg/day, by intraperitoneal injection) was then administered throughout the 14 day period. The mice also received a daily injection of bromo-deoxyuridine (BrdU) by intraperitoneal injection). The other two groups of mice), control and bleomycin-treated received a daily intraperitoneal injection of the vehicle containing BrdU. The vehicle comprised 10% dimethyl sulphoxide in peanut oil.

Bleomycin was administered on Day 1 as a single 35 pl intranasal droplet containing 125 mUnits dissolved in normal saline. The mice were first lightly anaesthetised with Penthrane inhalational anaesthetic. After application of the droplet to the nares and insufflation of the droplet by the mouse, the mouse was maintained at an angle of 45° to facilitate pulmonary distribution of the bleomycin.

Body weight was measured throughout the experiment. At the end of the experiment between 12 and 14 days after bleomycin administration, the mice were anaesthetised with a mixture of ketamine and xylazine and prepared for recording of respiratory resistance and compliance using a whole body plethysmograph (Buxco Inc.). The mice were mechanically ventilated at 150 breaths per minute with a tidal volume of 150 μL. After a 5 minute stabilization period, values of resistance and compliance were recorded and the animal was killed by a lethal dose of pentabarbitone.

Half of each group of animals was then subjected to bronchoalveolar lavage (BAL) to generate BAL fluid (BALF) in which the number of infiltrating cells was counted by haemocytometry. The lungs from one half of the group of animals were snap frozen. The lungs from the other half of the group of mice were fixed by infusion of buffered formal saline at a pressure of 25 cmH₂O for 20 min prior to transfer to a beaker containing the same fixative. These specimens were then processed into paraffin for later sectioning (5 micron sections) and analysis for morphology using haematoxylin and eosin staining by standard methods by workers that remained blinded to the treatment groups from which the specimens were obtained. In addition, sections were also stained for collagen using Masson's trichrome stain. Further sections were prepared for BrdU positive cell nuclei. The presence of this stain signifies that the cell has undergone DNA synthesis during the period over which the BrDU has been administered and it therefore serves as an index of cell proliferation.

Tissue areas reported in Table 6 were determined by capturing images of the lung section at a 100 times magnification and importing those images into a powerpoint file. An overlay grid with at least 10 intersection points covering the whole of the captured image was used to identify areas that were then inspected at 600× magnification and captured by Image pro software.

A 36 point overlay was then applied to the captured image and the number of grid points that intersect with tissue was determined for each of the 10 selected fields. The data presented in Table 6 represent the percentage of grid points falling on tissue.

The fibrosis score was determined by tracing the area of fibrosis as determined morphologically on low power (10× magnification) images and expressing this as a percentage of the total area of the section, comprising a cross section of the whole lung. Within the area of fibrosis an assessment of the severity of the fibrotic lesion was made using a 6 point scale in which 0 denotes normal lung tissue structure and 6 represents complete consolidation of airspaces with fibroblasts and inflammatory cell infiltrates. The data presented in Table 6 represent the mean of the percentage affected area times its severity grade.

Cell proliferation was determined in the major intra-parenchymal airways of each of two lung lobes, by measuring the number of BrdU positive nuclei in the major airway expressed as a percentage of the total number of nuclei. Cells were identified in the airway wall as epithelium or mesenchymal (smooth muscle or fibroblast) based on morphology and location. The data indicate that 4NO treatment selectively attenuates that proliferation of mesenchymal cells.

TABLE 6
Effect of treatment with 4NO (50 mg/kg/day, i.p.) on
bleomycin-induced fibrosis in C57BL6 female mice.

| | Control | Bleomycin | Bleo/4NO |
|---|---|---|---|
| Initial | 20.5 ± 0.3 | 20.7 ± 0.3 | 20.6 ± 0.3 |
| body weight | | | |
| (g, n = 11-12) | | | |
| Percent change | 6.4 ± 0.8* | −6.7 ± 2.6* | −1.7 ± 1.6 |
| in weight | | | |
| (g, n = 11-12) | | | |
| Resistance | 0.42 ± 0.01 | 0.50 ± 0.03* | 0.45 ± 0.01 |
| (cmH₂O/ml/s) | | | |
| (n = 11-12) | | | |
| Compliance | 0.104 ± 0.004 | 0.071 ± 0.008* | 0.080 ± 0.003* |
| (ml/cmH₂O) | | | |
| (n = 11-12) | | | |
| BALF cell # | 3926 ± 881 | 11463 ± 2130* | 8356 ± 1434 |
| (n = 5-6) | | | |
| Fibrosis | 0.00 ± 0.00 | 0.80 ± 0.17* | 0.10 ± 0.06 |
| score (n = 6) | | | |
| Tissue area | 31.1 ± 1.0 | 44.4 ± 2.3* | 35.0 ± 1.7 |
| (%) (n = 6) | | | |
| Proliferation | 0.6117 ± 0.1752 | 4.646 ± 0.8564* | 3.802 ± 0.9053 |
| Epithelial | | | |
| Mesenchymal | 1.036 ± 0.3941 | 10.16 ± 1.969* | 6.065 ± 1.223 |
| (n = 6) | | | |
| Parenchymal§ | 109 ± 31 | 1340 ± 287* | 738 ± 185 |
| Cell Prolif. | | | |

*P < 0.05, compared with control
= P < 0.05, compared with no change (ie., 0).
Data area presented as mean ± standard error of the mean
§data represents positive cells/10⁶ pixels at magnification of 600×.

The initial body weights of the mice in the three groups did not differ. Following the 12-14 day treatment period, control mice gained weight, bleomycin/vehicle mice lost weight and the group treated with bleomycin/4NO showed no significant change. Airway resistance increased significantly in mice treated with vehicle/bleomycin, but not in those treated with bleomycin/4NO. Compliance decreased significantly in bleomycin treated mice, irrespective of 4NO treatment. The number of inflammatory lymphocytes/leukocytes in the BALF increased significantly in mice treated with vehicle/bleomycin, but not in those treated with bleomycin/4NO. Both the fibrosis score and the area of tissue were increased in bleomycin/vehicle mice, but not in those treated with bleomycin/4NO.

Alternative animal models of fibrosis are also available, such as administration of carbonyl iron together with carbon tetrachloride which induces fibrosis in the liver of mice (Arezzini, Lunghi et al. 2003). Airway fibrosis in mice can be induced by repeated administration of ovalbumin in mice previously sensitised by intraperitoneal injection of ovalbumin (Blyth, Wharton et al. 2000; Kuhn, Horner et al. 2000; Wills-Karp 2001).

Pulmonary Inflammation

A model of pulmonary inflammation resulting from the administration of lipopolysaccharide (LPS) (Bozinvski, et al., Innate immune responses to LPS in mouse lung are suppressed and reversed by neutralization of GM-CSF via repression of TLR-4. Am J Physiol LCMP (2004) 286:L877-L885) was used to investigate the ability of certain disclosed compounds to inhibit pulmonary inflammation.

In order to ascertain the effect of on lung inflammation, female balb/c mice were pretreated by intraperitoneal injection of either the vehicle (as described above in the bleomycin experiments), CP-DM-4-35 or 4NO each at 150 mg/kg, 2 hours before administration of 35 μL of LPS (for a total of 1 μg of LPS) administered intranasally as described earlier. 24 hours after LPS administration, mice were killed with an overdose of sodium pentobarbitone, the trachea was cannulated to enable BAL to be obtained for the enumeration of inflammatory lymphocytes and leukocytes and to determine protein levels using a BioRad protein assay reagent kit. In addition, the amount of active MMP-9 in the BALF was determined by zymography using well-established methodologies (see for example, Johnson S, Knox A. Autocrine production of matrix metalloproteinase-2 is required for human airway smooth muscle proliferation Am J. Physiol. 1999 December; 277(6 Pt 1):L1109-17).

LPS (1 μg) increased BALF cell number significantly in each group although there was a trend for the cell number to be reduced by both 4NO and CP-DM-4-35 (Table 7). Protein levels in BALF increased in LPS/vehicle mice but not in those pretreated with 4NO or CP-DM-4-35 prior to LPS challenge. Active MMP-9 levels increased significantly in LPS/vehicle treated animals but not in those pretreated with 4NO or CP-DM-4-35 prior to LPS challenge.

In the light of the known anti-inflammatory activity of glucocorticoid compounds in lung inflammation, and the known ability of these compounds to also inhibit inflammation in other tissues, there is a reasonable expectation that compounds disclosed in the present application will have anti-inflammatory activity in other sites of the body.

TABLE 7
Effect of treatment with 4NO (150 mg/kg, i.p.) or CP-DM-4-35 (150
mg/kg, i.p.) on LPS-induced inflammation in female Balb/c mice.

| | Control | LPS(1 μg) | LPS/4NO | LPS/CP-DM-4-35 |
|---|---|---|---|---|
| BALF cell # | 12,578 ± 2,630 | 54,058 ± 7,872* | 38,565 ± 5,809* | 38,316 ± 5,906* |
| (n = 14) | | | | |
| BALF protein(mg) | 0.16 ± 0.02 | 0.26 ± 0.03* | 0.16 ± 0.02 | 0.20 ± 0.01 |
| (n = 14) | | | | |
| Active MMP-9 | 0.07 ± 0.02 | 0.46 ± 0.10* | 0.32 ± 0.08 | 0.30 ± 0.08 |
| Arbitrary units | | | | |
| (n = 6) | | | | |

*P < 0.05, compared with control
Data area presented as mean ± standard error of the mean

### Example 52

### Clinical Assessment of Smooth Muscle and Fibroblast Related Pathology

The clinical evaluation of asthma is well described in the recent literature. Novel anti-asthma agents are evaluated against current best practice treatment for the particular severity of asthma under consideration. In moderate asthma, a number of clinical endpoints would be used to evaluate a new therapy including the rate of use of rescue (short-acting beta-agonist) therapy; the number of exacerbations of asthma defined as requirement for short course of oral glucocorticoids or hospital admissions; FEV₁; symptom score based on diary card; quality of life; level of glucocorticoid-sparing without loss of asthma control (Lemanske, Sorkness et al. 2001; Lemanske, Nayak et al. 2002). A number of studies have specifically examined the remodelling component of asthma pathology in airway biopsies taken at the outset and the conclusion of trial therapy (Sont, Willems et al. 1999). In such biopsies it is feasible to measure the amount of smooth muscle and connective tissue, the fraction of cells showing markers of cellular proliferation and the degree of epithelial damage (Druilhe, Wallaert et al. 1998; Benayoun, Druilhe et al. 2003).

Restenosis

Restenosis can be assessed in clinical trials by a number of endpoints. Indirect outcomes include the incidence of myocardial infarction and incidence of angina. In addition the neointima can be measured directly by angiography using radiographic or ultrasound-based approaches (Topol, Califf et al. 1994; Brack, Ray et al. 1995; Leon, Baim et al. 1998; Serruys, Foley et al. 2001).

Pulmonary Fibrosis

The status of pulmonary fibrosis in clinical studies is established by clinical measures such as clubbing, radiographic abnormalities and pulmonary function testing (talmadge king). The relevance of these disease features to disease progression and their importance in evaluation of novel therapies has been established by consensus based on systematic review of the available literature (Lipinski, Black et al. 1975; King 2000; King, Tooze et al. 2001; Selman, King et al. 2001)).

Other Conditions Involving Smooth Muscle and Fibroblasts

A variety of well-established clinical protocols are available for the evaluation of novel agents in other conditions in which fibroblast and smooth muscle functions are altered. The skilled investigator will be able to access and implement these protocols by searching of public domain databases such as PubMed.

It will be understood to persons skilled in the art of the invention that many modifications may be made without departing from the spirit and scope of the invention.

For convenient reference, the following table lists the Compound ID numbers and their associated chemical names used throughout the specification

TABLE 8

| Compound ID | Chemical Name |
|---|---|
| | 2-methoxyestradiol (2MEO) |
| CP-DM-2-11-7 | 2-methoxy-6-(4-nitrobenzyloxy)iminoestradiol (4NO) |
| CP-DM-3-91 | 2-methoxy-6-(4-nitrobenzyloxy)iminoestrone-17- |
| | oxime(4NOM) |
| CP-DM-3-106 | 2-methoxy-6-(3-nitrobenzyloxy)iminoestradiol |
| CP-DM-3-105 | 2-methoxy-6-(3-nitrobenzyloxy)iminoestrone-17-oxime |
| CP-DM-3-124 | 2-methoxy-6-(2-nitrobenzyloxy)iminoestradiol |
| CP-DM-3-117 | 2-methoxy-6-(2-nitrobenzyloxy)iminoestrone-17-oxime |
| CP-DM-3-119 | 2-methoxy-6-(2,4-dinitrophenylhydrazono)estrone-17- |
| | oxime |
| CP-DM-3-118 | 2-methoxy-6-(3- |
| | trifluoromethylbenzyloxy)iminoestrone-17-oxime |
| CP-DM-4-15 | 2-methoxy-6-(4-pyridylmethyloxy)iminoestrone-17- |
| | oxime |
| CP-DM-4-16 | 2-methoxy-6-(4-pyridylmethyloxy)iminoestradiol |
| CP-DM-4-36 | 6-(3,5-difluorobenzyloxy)imino-2-methoxyestrone-17- |
| | oxime |
| CP-DM-4-35 | 6-(3,5-difluorobenzyloxy)imino-2-methoxyestradiol |
| CP-DM-4-37 | 6-(4-cyanobenzyloxy)imino-2-methoxyestradiol |
| CP-DM-4-51 | 2-methoxy-6-(3-cyanobenzyloxy)iminoestrone-17-oxime |
| CP-DM-4-52 | 2-methoxy-6-(3-cyanobenzyloxy)iminoestradiol |
| CP-DM-4-38 | 6-(4-cyanobenzyloxy)imino-2-methoxyestrone-17-oxime |
| CP-DM-3-101 | estrone-17-(4-nitrobenzyl)oxime |
| CP-DM-3-102 | estrone-17-(3-nitrobenzyl)oxime |
| CP-DM-3-103 | 2-methoxyestrone-17-(4-nitrobenzyl)oxime |
| CP-DM-3-104 | 2-methoxyestrone-17-(3-nitrobenzyl)oxime |
| CP-DM-4-5 | 2-methoxy-6-(4-methoxybenzyloxy)-iminoestradiol |
| CP-DM-4-6 | 2-methoxy-6-(4-methoxybenzyloxy)-iminoestrone-17- |
| | oxime |
| CP-DM-4-33 | estrone-17-(4-methoxybenzyl)oxime |
| CP-DM-4-34 | 2-methoxyestrone-17-(4-methoxybenzyl)oxime |
| CP-DM-4-65 | 2-methoxy-6-(4- |
| | trifluoromethylthiobenzyloxy)iminoestradiol |
| CP-DM-4-66 | 2-methoxy-6-(3-methoxybenzyloxy)iminoestrone-17- |
| | oxime |
| CP-DM-4-68 | 2-methoxy-6-(4- |
| | trifluoromethoxybenzyloxy)iminoestrone-17-oxime |
| CP-DM-4-62 | 2-methoxy-6-(3-methoxybenzyloxy)iminoestradiol |
| CP-DM-4-63 | 2-methoxy-6-(3- |
| | trifluoromethoxybenzyloxy)iminoestradiol |
| CP-DM-4-64 | 2-methoxy-6-(4- |
| | trifluoromethoxybenzyloxy)iminoestradiol |
| CP-DM-4-67 | 2-methoxy-6-(3- |
| | trifluoromethoxybenzyloxy)iminoestrone-17-oxime |
| CP-DM-4-69 | 2-methoxy-6-(4- |
| | trifluoromethylthiobenzyloxy)iminoestrone-17-oxime |
| CP-DM-4-74 | 6-(3,5difluorobenzyloxy)imino-2-methoxyestrone-17- |
| | methyloxime |
| CP-DM-4-75 | 6-(4-nitrobenzyloxy)imino-2-methoxyestrone-17- |
| | methyloxime |
| CP-DM-4-76 | 2-methoxy-6-(4-methylbenzyloxy)iminoestrone-17- |
| | oxime |
| CP-DM-4-77 | 2-methoxy-6-(4-isopropylbenzyloxy)iminoestrone-17- |
| | oxime |
| CP-DM-4-78 | 2-methoxy-6-(4-methylbenzyloxy)iminoestradiol |
| CP-DM-4-88 | 6-(3,5-difluorobenzyloxy)iminoestriol |
| CP-DM-4-91 | 2-Methoxy-6-(4-nitrobenzyloxy)iminoestradiol-17- |
| | acetate |
| CP-DM-4-89 | 6-(4-nitrobenzyloxy)imino-17-ethinyl-estradiol |

### REFERENCES

1. Hamor, G. H., D. M. Breslow, and G. W. Fisch, Benzyloxyamines as Possible Inhibitors of Histamine Biosynthesis. Journal of Pharmaceutical Sciences, 1970. 59(12): p. 1752-1756.
2. Petrassi, M. H., B. K. Sharpless, and J. W. Kelly, The Copper-Mediated Cross-Coupling of Phenylboronic Acids and N-Hydroxyphthalimide at Room Temperature: Synthesis of Aryloxyamines. Organic Letters, 2001. 3(1): p. 139-142.
3. Miyazawa, E., T. Sakamoto, and Y. Kikugawa, Preparation of Ring-Substituted Phenoxyamine Derivatives. Organic Preparations and procedures International; Oppi Briefs, 1997. 29(5): p. 594-600.
4. Tessier, J., et al., Hydroxylamine derivatives and their use as plant growth factors. 1984, Roussel-UCLAF, Fr: France FR 2541282.
5. Knutsen, L., U. B. Olsen, and A. N. Bowler, Preparation of nucleosides for treating disorders related to cytokines in mammals. 1997, Novo Nordisk A/S, Den: PCT Int. Appl. WO 9733591.
6. Zinner, G. and E. U. Ketz, Hydroxylamin-Derivate; 5. O-und N-(5-Nitro-2-pyridyl)-hydroxylamin. Synthesis, 1973. 3: p. 165-166.
7. Hylarides, M. D. and F. A. Mettler Jr, Synthesis of 1-halosteroids. 1984, The University of New Mexico: U.S. Pat. No. 4,676,932.
8. Clark, E. R., A. M. E. Omar, and G. Prestwich, Potential Steroidal Antiestrogens. Journal of Medicinal Chemistry, 1977. 20(8): p. 1096-1099.
9. Wachter, M. P. and J. A. Settepani, 1-Oxygenated Steroids. 1978, Ortho Pharmaceutical Corporation: U.S. Pat. No. 4,096,253.
10. Cantrall, E. W., R. B. Conrow, and S. Bernstein, 1-Substituted estratrienes. 1966, American Cyanamid Co.: U.S. Pat. No. 3,251,865.
11. Ali, H., et al., 2- and 4-Fluorinated 16α-[125I]Iodoestradiol Derivatives; Synthesis and Effect on Estrogen Receptor Binding and Receptor-Mediated Target Tissue Uptake. Journal of Medicinal Chemistry, 1993. 36(26): p. 4255-4263.
12. Lovely, C. J., et al., 2-(Hydroxyalkyl)oestradiols: Synthesis and Biological Evaluation. Journal of Medicinal Chemistry, 1996. 39(9): p. 1917-1923.
13. Hughes, R. A., et al., 2-Methoxyestradiol and Analogs as Novel Antiproliferative Agents: Analysis of Three-Dimensional Quantitative Structure-Activity Relationships for DNA Synthesis Inhibition and Estrogen Receptor Binding. Molecular Pharmacology, 2002. 61(5): p. 1053-1069.
14. Cushman, M., et al., Synthesis of Analogs of 2-Methoxyestradiol with Enhanced Inhibitory Effects on Tubulin Polymerization and Cancer Cell Growth. Journal of Medicinal Chemistry, 1997. 40(15): p. 2323-2334.
15. Cushman, M., et al., Synthesis, Antitubulin and Antimitotic Activity, and Cytotoxicity of Analogs of 2-Methoxyestradiol, An Endogenous Mammalian Metabolite of Estradiol That Inhibits Tubulin Polymerization by Binding to the Colchicine Binding Site. Journal of Medicinal Chemistry, 1995. 38(12): p. 2041-2049.
16. Cushman, M., et al., The Effect of Exchanging Various Substituents at the 2-Position of 2-Methoxyestradiol on Cytotoxicity in Human Cancer Cell Cultures and Inhibition of Tubulin Polymerization. Journal of Medicinal Chemistry, 2002. 45(21): p. 4748-4754.
17. Poirier, J. M. and C. Vottero, Mononitration de phenols par des nitrates metalliques. Tetrahedron, 1989. 45(5): p. 1415-1422.
18. Purohit, A., et al., The Effect of 2-Methoxyestron-3-O-Sulphamate on the Growth of Breast Cancer Cells and Inducede Mammary Tumours. International Journal of Cancer, 2000. 85: p. 584-589.
19. Riess, J. and G. Ourisson, Derives phosphores d'hormones steroides. Methyl phosphonates de steroides. Memoirres presentes a la societe chimique, 1965. 5: p. 933-941.
20. Woo, L. W. L., et al., Oestrone 3-O-(Acetyl)Sulphamate, A Potential Molecular Probe of the Active Site of Oestrone Sulphatase. Bioorganic and Medicinal Chemistry Letters, 1997. 7(24): p. 3075-3080.
21. Crabbe, p., L. A. Maldonado, and I. Sanchez, Synthesis of New Heterocyclic Derivatives of Estradiol. Tetrahedron, 1971. 27: p. 711-725.
22. Welch, S. C. and M. E. Walters, Reduction of Aryl Diethyl Phosphates with Titanium Metal: A Method for Deoxygenation of Phenols. Journal of Organic Chemistry, 1978. 43(25): p. 4797-4799.
23. Pert, D. J. and D. D. Ridley, Formylation of Oestrogens. Australian Journal of Chemistry, 1989. 42: p. 405-419.
24. Holton, P. G., Steroids. CLXXVI. Claisen Rearrangement of Estrone Allyl Ether. Journal of Pharmaceutical and Medicinal Chemistry, 1962: p. 357-361.
25. Lippert, C., et al., The Effects of A-Ring and D-Ring metabolites of Estradiol on the Proliferation of Vascular Endothelial Cells. Life Sciences, 2000. 67: p. 1653-1658.
26. Katzenellenbogen, J. A., H. N. Myers, and H. J. J. Johnson, Reagents for Photoaffinity Labeling of Estrogen Binding Proteins. Synthesis of some Azide and Diazo Derivatives of Estradiol, Estrone, and Hexestrol. Journal of Organic Chemistry, 1973. 38(20): p. 3525-3532.
27. Arterburn, J. B. and M. C. Perry, Selective Rhenium-Catalyzed Oxidation of Secondary Alcohols with Methyl Sulfoxide in the Presence of Ethylene Glycol, a Convenient One-Pot Synthesis of Ketals. Organic Letters, 1999. 1(5): p. 769-771.
28. Malachowski, W. P., et al., The Synthesis of Azapeptidomimetic,—Lactam Molecules as Potential Protease Inhibitors. Journal of Organic Chemistry, 2002. 67(25): p. 8962-8962.
29. Gardner, T. S., E. Wenis, and J. Lee, Monoamine oxidase inhibitors. II. Some amino-and dialkylaminobenzylhydrazines and their acyl derivatives. J. Med. Pharm. Chem., 1961. 3: p. 241-252.
30. Markaverich, B. M. & Clark, J. H. (1979). Two binding sites for estradiol in rat uterine nuclei: relationship to uterotropic response. Endocrinology, 105, 1458-62.
31. Williams, R. C., Jr. & Lee, J. C. (1982). Preparation of tubulin from brain. Methods Enzymol, 85, 376-85.
32. D'Amato, R. J., Lin, C. M., Flynn, E., Folkman, J. & Hamel, E. (1994). 2-Methoxyestradiol, an endogenous mammalian metabolite, inhibits tubulin polymerization by interacting at the colchicine site. Proc Natl Acad Sci USA, 91, 3964-8.
33. Penefsky, H. S. (1977). Reversible binding of Pi by beef heart mitochondrial adenosine triphosphatase. J Biol Chem, 252, 2891-9.
34. Smith, M. B; March, J. (2001) March's Advanced Organic Chemistry: Reactions, Mechanisms, and Structure; 5^{th} Edition; John Wiley and Sons, NY, USA.
35. Walter M J, Morton J D, Kajiwara N, Agapov E, Holtzman M J. (2002). Viral induction of a chronic asthma phenotype and genetic segregation from the acute response. J Clin Invest, 110(2), 165-75
36. Panettieri R A Jr. (2002). Airway smooth muscle: an immunomodulatory cell. J Allergy Clin Immunol, 110(6 Suppl), S269-74
37. Ritz, S A., Stampfli, M R., Davies, D E., Holgate, S T. & Jordana. M. (2002). On the generation of allergic airway diseases from GM-CSF to Kyoto. Trends in Immunology, 23, 396-402.
38. Hamilton, J A., (2002). GM-CSF in inflammation and autoimmunity. Trends in Immunology, 23, 403-408.
39. Gleich, G, J, & Kita, H. (1997). Bronchial asthma: lessons from murine models. Proceedings of National Academy of Science, USA, 94, 2101-2102.
40. Andersen, H. L., J. U. Weis, et al. (1999). “Effect of acute and long-term treatment with 17-beta-estradiol on the vasomotor responses in the rat aorta.” Br J Pharmacol 126(1): 159-68.
41. Arezzini, B., B. Lunghi, et al. (2003). “Iron overload enhances the development of experimental liver cirrhosis in mice.” Int J Biochem Cell Biol 35(4): 486-95.
42. Armour, C. L., N. M. Lazar, et al. (1984). “A comparison of in vivo and in vitro human airway reactivity to histamine.” Am Rev Respir Dis 129(6): 907-10.
43. Arthur, J. F., Z. L. Yin, et al. (1997). “Induction of nitric oxide synthase in the neointima induced by a periarterial collar in rabbits.” Arterioscler Thromb Vasc Biol 17(4): 737-40.
44. Benayoun, L., A. Druilhe, et al. (2003). “Airway structural alterations selectively associated to severe asthma.” Am J Respir Crit Care Med.
45. Berk, B. C., E. Elder, et al. (1990). Hypertrophy and hyperplasia cause differing effects on vascular smooth muscle cell Na+/H+ exchange and intracellular pH. J Biol Chem. 265: 19632-7.
46. Bishop, J. E., J. J. Mitchell, et al. (1993). “Cyclic mechanical deformation stimulates human lung fibroblast proliferation and autocrine growth factor activity.” Am J Respir Cell Mol Biol 9(2): 126-33.
47. Blennerhassett, M. G., F. M. Bovell, et al. (1999). “Characteristics of inflammation-induced hypertrophy of rat intestinal smooth muscle cell.” Dig Dis Sci 44(7): 1265-72.
48. Blyth, D. I., T. F. Wharton, et al. (2000). “Airway subepithelial fibrosis in a murine model of atopic asthma: suppression by dexamethasone or anti-interleukin-5 antibody.” Am J Respir Cell Mol Biol 23(2): 241-6.
49. Brack, M. J., S. Ray, et al. (1995). “The Subcutaneous Heparin and Angioplasty Restenosis Prevention (SHARP) trial. Results of a multicenter randomized trial investigating the effects of high dose unfractionated heparin on angiographic restenosis and clinical outcome.” J Am Coll Cardiol 26(4): 947-54.
50. Butt, R. P., G. J. Laurent, et al. (1995). “Collagen production and replication by cardiac fibroblasts is enhanced in response to diverse classes of growth factors.” Eur J Cell Biol 68(3): 330-5.
51. Campbell, J. H. and G. R. Campbell (1993). “Culture techniques and their applications to studies of vascular smooth muscle.” Clin. Sci. (Colch.) 85(5): 501-513.
52. Chang, W. C., J. Nakao, et al. (1980). “Stimulation of prostaglandin cyclooxygenase and prostacyclin synthetase activities by estradiol in rat aortic smooth muscle cells.” Biochim Biophys Acta 620(3): 472-82.
53. Dominiczak, A. F., A. M. Devlin, et al. (1997). “Left ventricular hypertrophy and arterial blood pressure in experimental models of hypertension.” Adv Exp Med Biol 432: 23-33.
54. Druilhe, A., B. Wallaert, et al. (1998). “Apoptosis, proliferation, and expression of Bcl-2, Fas, and Fas ligand in bronchial biopsies from asthmatics.” Am J Respir Cell Mol Biol 19(5): 747-57.
55. Dube, J., J. Chakir, et al. (1998). In vitro procollagen synthesis and proliferative phenotype of bronchial fibroblasts from normal and asthmatic subjects. Lab Invest. 78: 297-307.
56. Evans, T. W., R. J. McAnulty, et al. (1990). “Bleomycin-induced lung injury in the rat: effects of the platelet-activating factor (PAF) receptor antagonist BN 52021 and platelet depletion.” Environ Health Perspect 85: 65-9.
57. Fernandes, D., E. Guida, et al. (1999). “Glucocorticoids inhibit proliferation, cyclin D1 expression and retinoblastoma protein phosphorylation, but not activity of the extracellular regulated kinases (ERK) in human cultured airway smooth muscle.” Am J Respir Cell Mol Biol 21: 77-88.
58. Gerin, W., T. G. Pickering, et al. (2000). “An historical context for behavioral models of hypertension.” J Psychosom Res 48(4-5): 369-77.
59. Haff, R. C., C. P. Page, et al. (1977). “Splenectomy: its place in operations for inflammatory disease of the pancreas.” Am J Surg 134(5): 555-7.
60. Hickey, H., M. Makdissi, et al. (1996). “Perindopril treatment prevents the loss of endothelial nitric oxide function and development of neo-intima in rabbits.” J Mol Cell Cardiol 28(9): 1985-94.
61. Hodgin, J. B. and N. Maeda (2002). “Minireview: estrogen and mouse models of atherosclerosis.” Endocrinology 143(12): 4495-501.
62. Hupfeld, C. J. and R. H. Weiss (2001). “TZDs inhibit vascular smooth muscle cell growth independently of the cyclin kinase inhibitors p21 and p27.” Am J Physiol Endocrinol Metab 281(2): E207-16.
63. Isaeff, M., L. Goya, et al. (1993). “Alterations in the growth and protein content of human neuroblastoma cells in vitro induced by thyroid hormones, stress and ageing.” Journal of Reproduction & Fertility—Supplement 46: 21-33.
64. Jeffery, T. K. and J. C. Wanstall (2001). “Pulmonary vascular remodeling: a target for therapeutic intervention in pulmonary hypertension.” Pharmacol Ther 92(1): 1-20.
65. John, M., B. T. Au, et al. (1998). “Expression and release of interleukin-8 by human airway smooth muscle cells: inhibition by Th-2 cytokines and corticosteroids.” Am. J. Respir. Cell Mol. Biol. 18(1): 84-90.
66. Karas, R. H. (2002). “Animal models of the cardiovascular effects of exogenous hormones.” Am J Cardiol 90(1A): 22F-25F.
67. King, T. E., Jr. (2000). “Interferon gamma-1b for the treatment of idiopathic pulmonary fibrosis.” N Engl J Med 342(13): 974-5.
68. King, T. E., Jr., J. A. Tooze, et al. (2001). “Predicting survival in idiopathic pulmonary fibrosis: scoring system and survival model.” Am J Respir Crit Care Med 164(7): 1171-81.
69. Kuhn, C., 3rd, R. J. Horner, et al. (2000). “Airway hyperresponsiveness and airway. obstruction in transgenic mice. Morphologic correlates in mice overexpressing interleukin (IL)-11 and IL-6 in the lung.” Am J Respir Cell Mol Biol 22(3): 289-95.
70. Lemanske, R. F., Jr., A. Nayak, et al. (2002). “Omalizumab improves asthma-related quality of life in children with allergic asthma.” Pediatrics 110(5): e55.
71. Lemanske, R. F., Jr., C. A. Sorkness, et al. (2001). “Inhaled corticosteroid reduction and elimination in patients with persistent asthma receiving salmeterol: a randomized controlled trial.” Jama 285(20): 2594-603.
72. Leon, M. B., D. S. Baim, et al. (1998). “A clinical trial comparing three antithrombotic-drug regimens after coronary-artery stenting. Stent Anticoagulation Restenosis Study Investigators.” N Engl J Med 339(23): 1665-71.
73. Lipinski, D., J. L. Black, et al. (1975). “Influence of motivational variables on the reactivity and reliability of self-recording.” J Consult Clin Psychol 43(5): 637-46.
74. Mattingly, R. R., R. A. Gibbs, et al. (2002). “Potent suppression of proliferation of a10 vascular smooth muscle cells by combined treatment with lovastatin and 3-allylfarnesol, an inhibitor of protein farnesyltransferase.” J Pharmacol Exp Ther 303(1): 74-81.
75. McKay, S., J. C. de Jongste, et al. (1998). “Angiotensin II induces hypertrophy of human airway smooth muscle cells: expression of transcription factors and transforming growth factor-beta1.” Am J Respir Cell Mol Biol 18(6): 823-33.
76. Murota, H., Y. Hamasaki, et al. (2003). “Disruption of tumor necrosis factor receptor p55 impairs collagen turnover in experimentally induced sclerodermic skin fibroblasts.” Arthritis Rheum 48(4): 1117-25.
77. Nili, N., M. Zhang, et al. (2002). “Biochemical analysis of collagen and elastin synthesis in the balloon injured rat carotid artery.” Cardiovasc Pathol 11(5): 272-6.
78. Ramos, C., M. Montano, et al. (2001). “Fibroblasts from idiopathic pulmonary fibrosis and normal lungs differ in growth rate, apoptosis, and tissue inhibitor of metalloproteinases expression.” Am J Respir Cell Mol Biol 24(5): 591-8.
79. Rekhter, M. D. (2002). “How to evaluate plaque vulnerability in animal models of atherosclerosis?” Cardiovasc Res 54(1): 36-41.
80. Sechi, L. A. (1999). “Mechanisms of insulin resistance in rat models of hypertension and their relationships with salt sensitivity.” J Hypertens 17(9): 1229-37.
81. Selman, M., T. E. King, et al. (2001). “Idiopathic pulmonary fibrosis: prevailing and evolving hypotheses about its pathogenesis and implications for therapy.” Ann Intern Med 134(2): 136-51.
82. Serruys, P. W., D. P. Foley, et al. (2001). “The TRAPIST Study. A multicentre randomized placebo controlled clinical trial of trapidil for prevention of restenosis after coronary stenting, measured by 3-D intravascular ultrasound.” Eur Heart J 22(20): 1938-47.
83. Sont, J. K., L. N. Willems, et al. (1999). “Clinical control and histopathologic outcome of asthma when using airway hyperresponsiveness as an additional guide to long-term treatment. The AMPUL Study Group.” Am J Respir Crit Care Med 159(4 Pt 1): 1043-51.
84. Stoll, M. and H. J. Jacob (2001). “Genetic rat models of hypertension: relationship to human hypertension.” Curr Hypertens Rep 3(2): 157-64.
85. Sugiyama, F., K. Yagami, et al. (2001). “Mouse models of blood pressure regulation and hypertension.” Curr Hypertens Rep 3(1): 41-8.
86. Tani, K., S. Yasuoka, et al. (1991). “Thrombin enhances lung fibroblast proliferation in bleomycin-induced pulmonary fibrosis.” Am. J. Respir. Cell Mol. Biol. 5(1): 34-40.
87. Tomlinson, P. R., K. Croft, et al. (1994). “Platelet-activating factor biosynthesis in rat vascular smooth muscle cells.” J Vasc Res 31(3): 144-52.
88. Topol, E. J., R. M. Califf, et al. (1994). “Randomised trial of coronary intervention with antibody against platelet IIb/IIIa integrin for reduction of clinical restenosis: results at six months. The EPIC Investigators.” Lancet 343(8902): 881-6.
89. Uhal, B. D., C. Ramos, et al. (1998). “Cell size, cell cycle, and alpha-smooth muscle actin expression by primary human lung fibroblasts.” Am J Physiol 275(5 Pt 1): L998-L1005.
90. Van Put, D. J., C. E. Van Hove, et al. (1995). “Dexamethasone influences intimal thickening and vascular reactivity in the rabbit collared carotid artery.” European Journal of Pharmacology 294(2-3): 753-761.
91. Wanstall, J. C., A. Gambino, et al. (2002). “Vascular endothelial growth factor-B-deficient mice show impaired development of hypoxic pulmonary hypertension.” Cardiovasc Res 55(2): 361-8.
92. Wills-Karp, M. (2001). “IL-12/IL-13 axis in allergic asthma.” J Allergy Clin Immunol 107(1): 9-18.
93. Marinese, D., Patel, R. et al (2003) “Mechanistic Investigation of the Adrenergic Induction of Ventral Prostate Hyperplasia in Mice” Prostate 54 (3): 230-237.
94. Underwood et al., SB 239063, a p38 MAPK inhibitor, reduces neutrophilia, inflammatory cytokines, MMP-9, and fibrosis in lung. Am J Physiol Lung Cell Mol Physiol (2000) 279:L895-902.

1. A compound of the Formula I: 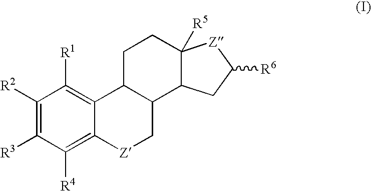
in which:
R¹ and R⁴ are each selected from the group consisting of H, R^{a}, —R^{c}R^{d}, —CN, —NO₂, -halo, OH, —OR^{a}, —OC(O)R^{a};
R² is selected from the group consisting of —OR^{b}, —(R^{c})ₙ-AR^{b}, —H, —R^{e}, —R^{c}R^{e}, —CH═NOH, —CH═NOR^{b}, —CH═NNR^{b}₂, —OH, —SR^{b}, —R^{b}, —CN, —R^{c}R^{d} and -halo, in which n is 0 or 1;
R³ is selected from the group consisting of —OH, —OR^{a}, —R^{c}OR^{b}, —H, -ester-R^{b};
R⁵ is methyl;
R⁶ is —H, —OH, —OR^{b} or -halo;
Z′ is A or >CH₂, >C═O, >C═N—OH, >C═N—OR^{b}, >C(R^{b})—OH, >C(R^{b})—CN; >C(R^{b})—NR^{b}₂, >CR^{b}₂, >C═N—NH₂, >C═N—NR^{b}₂, —O—, >N—R^{b}, >C(R^{b})—R^{c}—OR^{b}, >CR^{b}R^{e}, >CR^{b}—NR^{b}R^{e}, >C═N-ester-R^{a};
Z″ is A or >C═O, >C(H)OH, >C═N—OH, >C═N—OR^{b}, >C(R^{b})—OR^{b}, >C(R^{b})—R^{c}—OR^{b}, >C(H)—NR^{b}₂, >C(H)-halo, >CR^{b}₂, >C═N-ester-R^{a};
A is >C═N—O—X, >C═N—O—R^{c}—X, >C═N—NH—R^{c}—X, >C═N—NH—X, >C═N-ester-X;
X is an aromatic group substituted by one or more substituents Y, wherein Y is selected from —H, —NO₂, —CN, —SO₃H, —SO₃R^{a}, —CO—R^{b}, —⁺NR^{b}₃, —CO₂R^{b}, -halo, —CF₃, —CCl₃, tetrazole, imidazole, -aryl, -substituted aryl, —R^{a}, —NH₂, —NR^{a}₂, —OH, —OR^{a}, —R^{c}—CN, —R^{c}-halo, —NR^{b}COR^{b}, —R^{c}—NR^{b}₂, —R^{c}R^{d};
R^{a} is a straight chained, branched or cyclic alkyl, alkenyl, alkynyl, aralkyl, aralkenyl or aralkynyl;
R^{b} is H or straight chained, branched or cyclic alkyl, alkenyl, alkynyl, aralkyl, aralkenyl or aralkynyl;
R^{c} is a straight chained or branched C1-C10 alkylene, alkenylene or alkynylene;
R^{d} represents one or more substituents selected from —OH, —NH₂, -halo, —CF₃, —CN, —COOR^{a}, —SR^{b};
R^{e} is acyl; and
n is 0 or 1;
with the proviso the compound contains at least one group A,
or a salt, hydrate, pro-drug, isomer, tautomer and/or derivative thereof.

2. The compound of claim 1, wherein at least one of Z′ and Z″ is A.

3. The compound of claim 1, wherein X is selected from aryl groups substituted by one or more substituents Y¹, wherein Y¹ is selected from —NO₂, —CN, —SO₃H, —SO₃R^{a}, —CO—R^{b}, —⁺NR^{b}₃, —CO₂R^{b}, -halo, —CF₃, —CCl₃, tetrazole, imidazole, -aryl, -substituted aryl, —R^{a}, —NH₂, —NR^{a}₂, —OH, —OR^{a}, —R^{c}—CN, —R^{c}-halo, —NR^{b}COR^{b}, —R^{c}—NR^{b}₂, R^{c}R^{d}, and heteroaromatic groups substituted by one or more substituents Y, wherein Y is selected from —H, —NO₂, —CN, —SO₃H, —SO₃R^{a}, —CO—R^{b}, —⁺NR^{b}₃, —CO₂, -halo, —CF₃, —CCl₃, tetrazole, imidazole, -aryl, -substituted aryl, —R^{a}, —NH₂, —NR^{a}₂, —OH, —OR^{a}, —R^{c}—CN, —R^{c}-halo, —NR^{b}COR^{b}, —R^{c}—NR^{b}₂, —R^{c}R^{d}.

4. A compound of claim 1, which has activity in modulating smooth muscle cell and/or fibroblast function.

5. A compound according to claim 4, which has specific activity in modulating smooth muscle cell and/or fibroblast function.

6. A compound according to claim 4 or claim 5, in which the modulation of cell function is suppression of cell proliferation.

7. A compound according to claim 4 or claim 5, in which the modulation of cell function is modulating cell extracellular matrix deposition.

8. A compound according to claim 4 or claim 5, in which the modulation of cell function is modulating cell cytokine expression.

9. A compound according to claim 8, in which the modulation of cell cytokine expression is the suppression of GM-CSF expression.

10. A compound according to claim 4 or claim 5, in which the modulation of cell function is modulating cell contractility.

11. A compound according to claim 4 or claim 5, in which the modulation of cell function is the modulation of cell migration.

12. A compound according to claim 4 or claim 5, in which the smooth muscle cells are airway smooth muscle cells.

13. A compound according to claim 4 or claim 5, in which the fibroblasts are airway fibroblasts.

14. A compound according to claim 1 which has activity in suppressing airway hyperresponsiveness.

15. A compound according to claim 14 in which the airway hyperresponsiveness is associated with asthma.

16. A compound according to claim 1 which has activity in suppressing fibrosis.

17. A compound according to claim 1 which has activity in suppressing pulmonary fibrosis.

18. A compound according to claim 1 which has activity in suppressing inflammation.

19. The compound of claim 1, wherein Y is preferably a deactivating group selected from the group —NO₂, —CN, —SO₃H, —SO₃R^{a}, —CO—R^{b}, —⁺NR^{b}₃, —CO₂R^{b}, -halo, —CF₃—CCl₃, tetrazole and imidazole.

20. The compound of claim 19, wherein Y is selected from the group —NO₂, —CN, —CO₂R^{b}, -halo, —CF₃—CCl₃, tetrazole and imidazole.

21. The compound of claim 1, wherein X is selected from the group phenyl, naphthyl or pyridyl.

22. The compound of claim 1, wherein the group R^{c} in A is alkylene.

23. The compound of claim 1, wherein A is >C—N—O—X, >C═N—O—R^{c}—X or >C═N—NH—R^{c}—X.

24. The compound of claim 1, wherein Z″ is A and Z′ is selected from the group >CH₂, >C═O, >C═N—OH and >C═N—OR^{b}.

25. The compound of claim 1, wherein Z′ is A and Z″ is selected from the group >C═O, >C(H)OH, >C═N—OH and >C═N—OR^{b}.

26. The compound of claim 1, wherein R² is selected from the group —OR^{b}, -AR^{b}, —R^{e}, —R^{c}R^{e}, —CH═NOH, —CH═NOR^{b}, —CH═NNR^{b}₂, —OH, —SR^{b}, —R^{b} and —CN.

27. The compound of claim 25, wherein R² is —OMe.

28. The compound of claim 1, wherein R³ is selected from the group —OH, —OR^{a} and —R^{c}OR^{b}.

29. The compound of claim 1, wherein R¹ and R⁴ are each H.

30. The compound of claim 1, wherein R⁶ is H.

31. A compound of Formula (V): 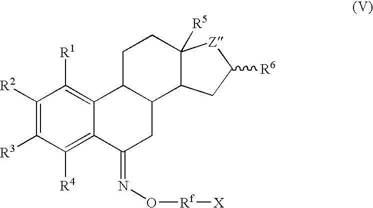
in which
R¹ and R⁴ are each selected from the group consisting of H, R^{a}, —R^{c}R^{d}, —CN, —NO₂, -halo, OH, —OR^{a}, —OC(O)R^{a};
R² is selected from the group consisting of —OR^{b}, —(R^{c})ₙ-AR^{b}, —H, —R^{e}, —R^{c}R^{e}, —CH═NOH, —CH═NOR^{b}, —CH═NNR^{b}₂, —OH, —SR^{b}, R^{b}, —CN, —R^{c}R^{d} and -halo, in which n is 0 or 1;
R³ is selected from the group consisting of —OH, —OR^{a}, R^{c}OR^{b}, —H, -ester-R^{b};
R⁵ is methyl;
R⁶ is —H, —OH, —OR^{b} or -halo;
Z″ is A or >C═O, >C(H)OH, >C═N—OH, >C═N—OR^{b}, >C(e)—OR^{b}, >C(R^{b})—R^{c}—OR^{b}, >C(H)—NR^{b}₂, >C(H)-halo, >CR^{b}₂, >C═N-ester-R^{a};
A is >C═N—O—X, >C═N—O—R^{c}—X, >C═N—NH—R^{c}—X, >C═N—NH—X, >C═N-ester-X;
X is an aromatic group substituted by one or more substituents Y, wherein Y is selected from —H, —NO₂, —CN, —SO₃H, —SO₃R^{a}, —CO—R^{b}, —⁺NR^{b}₃, —CO₂R^{b}, -halo, —CF₃, —CCl₃, tetrazole, imidazole, -aryl, -substituted aryl, —R^{a}, —NH₂, —NR^{a}₂, —OH, —OR^{a}, —R^{c}—CN, —R^{c}-halo, —NR^{b}COR^{b}, —R^{c}—NR^{b}₂, —R^{c}R^{d};
R^{a} is a straight chained, branched or cyclic alkyl, alkenyl, alkynyl, aralkyl, aralkenyl or aralkynyl;
R^{b} is H or straight chained, branched or cyclic alkyl, alkenyl, alkynyl, aralkyl, aralkenyl or aralkynyl;
R^{c} is a straight chained or branched C1-C10 alkylene, alkenylene or alkynylene;
R^{d} represents one or more substituents selected from —OH, —NH₂, -halo, —CF₃, —CN, —COOR^{a}, —SR^{b};
R^{e} is acyl; and
R^{f} is a direct bond or an alkylene group,
or a salt, hydrate, pro-drug, isomer, tautomer and/or derivative thereof.

32. A compound of Formula (VI): 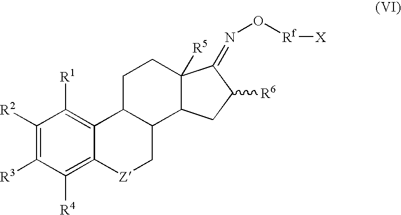
in which
R¹ and R⁴ are each selected from the group consisting of H, R^{a}, —R^{c}R^{d}, —CN, —NO₂, -halo, OH, —OR^{a}, —OC(O)R^{a};
R² is selected from the group consisting of —OR^{b}, —(R^{c})ₙ-AR^{b}, —H, —R^{e}, —R^{c}R^{e}, —CH═NOH, —CH═NOR^{b}, —CH═NNR^{b}₂, —OH, —SR^{b}, —R^{b}, —CN, —R^{c}R^{d} and -halo, in which n is 0 or 1;
R³ is selected from the group consisting of —OH, —OR^{a}, R^{c}OR^{b}, —H, -ester-R^{b};
R⁵ is methyl;
R⁶ is —H, —OH, —OR^{b} or -halo;
Z′ is A or >CH₂, >C═O, >C═N—OH, >C═N—OR^{b}, >C(R^{b})—OH, >C(R^{b})—CN; >C(R^{b})—NR^{b}₂, >CR^{b}₂, >C═N—NH₂, >C═N—NR^{b}₂, —O—, >N—R^{b}, >C(R^{b})—R^{c}—OR^{b}, >CR^{b}R^{e}, >CR^{b}—NR^{b}R^{e}, >C═N-ester-R^{a};
A is >C═N—O—X, >C═N—O—R^{c}—X, >C═N—NH—R^{c}—X, >C═N—NH—X, >C═N-ester-X;
X is an aromatic group substituted by one or more substituents Y, wherein Y is selected from —H, —NO₂, —CN, —SO₃H, —SO₃R^{a}, —CO—R^{b}, —⁺NR^{b}₃, —CO₂R^{b}, -halo, —CF₃, —CCl₃, tetrazole, imidazole, -aryl, -substituted aryl, —R^{a}, —NH₂, —NR^{a}₂, —OH, —OR^{a}, —R^{c}—CN, —R^{c}-halo, —NR^{b}COR^{b}, —R^{c}—NR^{b}₂, —R^{c}R^{d};
R^{a} is a straight chained, branched or cyclic alkyl, alkenyl, alkynyl, aralkyl, aralkenyl or aralkynyl;
R^{b} is H or straight chained, branched or cyclic alkyl, alkenyl, alkynyl, aralkyl, aralkenyl or aralkynyl;
R^{c} is a straight chained or branched C1-C10 alkylene, alkenylene or alkynylene;
R^{d} represents one or more substituents selected from —OH, —NH₂, -halo, —CF₃, —CN, —COOR^{a}, —SR^{b};
R^{e} is acyl; and
R^{f} is a direct bond or an alkylene group;
or a salt, hydrate, pro-drug, isomer, tautomer and/or derivative thereof.

33. A compound of Formula (VII): 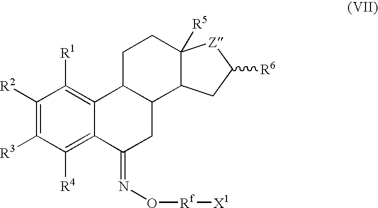
in which
R¹ and R⁴ are each selected from the group consisting of H, R^{a}, —R^{c}R^{d}, —CN, —NO₂, -halo, OH, —OR^{a}, —OC(O)R^{a};
R² is selected from the group consisting of —OR^{b}, —(R^{c})ₙ-AR^{b}, —H, R^{e}, R^{c}R^{e}, —CH═NOH, —CH═NOR^{b}, —CH═NNR^{b}₂, —OH, —SR^{b}, —R^{b}, —CN, —R^{c}R^{d} and -halo, in which n is 0 or 1;
R³ is selected from the group consisting of —OH, —OR^{a}, —R^{c}OR^{b}, —H, -ester-R^{b};
R⁵ is methyl;
R⁶ is —H, —OH, —OR^{b} or -halo;
Z″ is A or >C═O, >C(H)OH, >C═N—OH, >C═N—OR^{b}, >C(R^{b})—OR^{b}, >C(R^{b})—R^{c}—OR^{b}, >C(H)—NR^{b}₂, >C(H)-halo, >CR^{b}₂, >C═N-ester-R^{a};
A is >C═N—O—X, >C═N—O—R^{c}—X, >C═N—NH—R^{c}—X, >C═N—NH—X, >C═N-ester-X;
X is an aromatic group substituted by one or more substituents Y, wherein Y is selected from —H, —NO₂, —CN, —SO₃H, —SO₃R^{a}, —CO—R^{b}, —⁺NR^{b}₃, —CO₂R^{b}, -halo, —CF₃, —CCl₃, tetrazole, imidazole, -aryl, -substituted aryl, —R^{a}, —NH₂, —NR^{a}₂, —OH, —OR^{a}, —R^{c}—CN, —R^{c}-halo, —NR^{b}COR^{b}, —R^{c}—NR^{b}₂, —R^{c}R^{d};
R^{a} is a straight chained, branched or cyclic alkyl, alkenyl, alkynyl, aralkyl, aralkenyl or aralkynyl;
R^{b} is H or straight chained, branched or cyclic alkyl, alkenyl, alkynyl, aralkyl, aralkenyl or aralkynyl;
R^{c} is a straight chained or branched C1-C10 alkylene, alkenylene or alkynylene;
R^{d} represents one or more substituents selected from —OH, —NH₂, -halo, —CF₃, —CN, —COOR^{a}, SR^{b};
R^{e} is acyl;
R^{e} is a direct bond or an alkylene group, and
X¹ is selected from aryl groups substituted by one or more substituents Y¹, wherein Y¹ is selected from —NO₂, —CN, —SO₃H, —SO₃R^{a}, —CO—R^{b}, —⁺NR^{b}₃, —CO₂R^{b}, -halo, —CF₃, —CCl₃, tetrazole, imidazole, -aryl, -substituted aryl, —R^{a}, —NH₂, —NR^{a}₂, —OH, —OR^{a}, —R^{c}—CN, —R^{c}-halo, —NR^{b}COR^{b}, —R^{c}—NR^{b}₂, —R^{c}R^{d} and heteroaromatic groups substituted by one or more substituents Y,
or a salt, hydrate, pro-drug, isomer, tautomer and/or derivative thereof.

34. A compound of Formula (VIII): 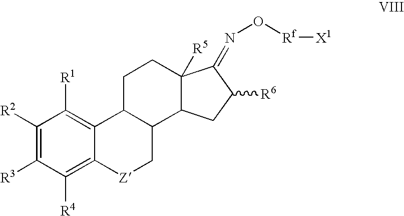
in which
R¹ and R⁴ are each selected from the group consisting of H, R^{a}, —R^{c}R^{d}, —CN, —NO₂, -halo, OH, —OR^{a}, —OC(O)R^{a};
R² is selected from the group consisting of —OR^{b}, —(R^{c})ₙ-AR^{b}, —H, —R^{e}, —R^{c}R^{e}, —CH═NOH, —CH═NOR^{b}, —CH═NNR^{b}₂, —OH, —SR^{b}, —R^{b}, —CN, —R^{c}R^{d} and -halo, in which n is 0 or 1;
R³ is selected from the group consisting of —OH, —OR^{a}, —R^{c}OR^{b}, —H, -ester-R^{b};
R⁵ is methyl;
R⁶ is —H, —OH, —OR^{b} or -halo;
Z′ is A or >CH₂, >C═O, >C═N—OH, >C═N—OR^{b}, >C(R^{b})—OH, >C(R^{b})—CN; >C(R^{b})—NR^{b}₂, >CR^{b}₂, >C═N—NH₂, >C═N—NR^{b}₂, —O—, >N—R^{b}, >C(R^{b})—R^{c}—OR^{b}, >CR^{b}R^{e}, >CR^{b}—NR^{b}R^{e}, >C═N-ester-R^{a};
A is >C═N—O—X, >C═N—O—R^{c}—X, >C═N—NH—R^{c}—X, >C═N—NH—X, >C═N-ester-X;
X is an aromatic group substituted by one or more substituents Y, wherein Y is selected from —H, —NO₂, —CN, —SO₃H, —SO₃R^{a}, —CO—R^{b}, —⁺NR^{b}₃, —CO₂R^{b}, -halo, —CF₃, —CCl₃, tetrazole, imidazole, -aryl, -substituted aryl, —R^{a}, —NH₂, —NR^{a}₂, —OH, —OR^{a}, —R^{c}—CN, —R^{c}-halo, —NR^{b}COR^{b}, —R^{c}—NR^{b}₂, —R^{c}R^{d};
R^{a} is a straight chained, branched or cyclic alkyl, alkenyl, alkynyl, aralkyl, aralkenyl or aralkynyl;
R^{b} is H or straight chained, branched or cyclic alkyl, alkenyl, alkynyl, aralkyl, aralkenyl or aralkynyl;
R^{c} is a straight chained or branched C1-C10 alkylene, alkenylene or alkynylene;
R^{d} represents one or more substituents selected from —OH, —NH₂, -halo, —CF₃, —CN, —COOR^{a}, —SR^{b};
R^{e} is acyl;
R^{f} is a direct bond or an alkylene group, and
X¹ is selected from aryl groups substituted by one or more substituents Y¹, wherein Y¹ is selected from —NO₂, —CN, —SO₃H, SO₃R^{a}, —CO—R^{b}, —⁺NR^{b}₃, —CO₂R^{b}, -halo, —CF₃, —CCl₃, tetrazole, imidazole, -aryl, -substituted aryl, —R^{a}, —NH₂, —NR^{a}₂, —OH, —OR^{a}, —R^{c}—CN, —R^{c}-halo, —NR^{b}COR^{b}, —R^{c}—NR^{b}₂, —R^{c}R^{d}, and heteroaromatic groups substituted by one or more substituents Y,
or a salt, hydrate, pro-drug, isomer, tautomer and/or derivative thereof.

35. A compound of Formula (IX): 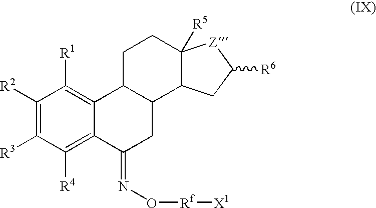
in which
R¹ and R⁴ are each selected from the group consisting of H, R^{a}, —R^{c}R^{d}, —CN, —NO₂, -halo, OH, —OR^{a}, —OC(O)R^{a};
R² is selected from the group consisting of —OR^{b}, —(R^{c})ₙ-AR^{b}, —H, —R^{e}, —R^{c}R^{e}, —CH═NOH, —CH═NOR^{b}, —CH═NNR^{b}₂, —OH, —SR^{b}, —R^{b}, —CN, —R^{c}R^{d} and -halo, in which n is 0 or 1;
R³ is selected from the group consisting of —OH, —OR^{a}, R^{c}OR^{b}, —H, -ester-R^{b};
R⁵ is methyl;
R⁶ is —H, —OH, —OR^{b} or -halo;
X¹ is selected from aryl groups substituted by one or more substituents Y¹, wherein Y¹ is selected from —NO₂, —CN, —SO₃H, SO₃R^{a}, —CO—R^{b}, —⁺NR^{b}₃, —CO₂R^{b}, -halo, —CF₃, —CCl₃, tetrazole, imidazole, -aryl, -substituted aryl, —R^{a}, —NH₂, —NR^{a}₂, —OH, —OR^{a}, —R^{c}—CN, R^{c}-halo, —NR^{b}COR^{b}, —R^{c}—NR^{b}₂, —R^{c}R^{d}; and heteroaromatic groups substituted by one or more substituents Y, wherein Y is selected from —H, —NO₂, —CN, —SO₃H, —SO₃R^{a}, —CO—R^{b}, —⁺NR^{b}₃, —CO₂R^{b}, -halo, —CF₃, —CCl₃, tetrazole, imidazole, -aryl, -substituted aryl, —R^{a}, —NH₂, —NR^{a}₂, —OH, —OR^{a}, —R^{c}—CN, —R^{c}-halo, —NR^{b}COR^{b}, —R^{c}—NR^{b}₂, —R^{c}R^{d};
Z′″ is >C═O, >C(H)OH or >C═N—OH;
R^{a} is a straight chained, branched or cyclic alkyl, alkenyl, alkynyl, aralkyl, aralkenyl or aralkynyl;
R^{b} is H or straight chained, branched or cyclic alkyl, alkenyl, alkynyl, aralkyl, aralkenyl or aralkynyl;
R^{c} is a straight chained or branched C1-C10 alkylene, alkenylene or alkynylene;
R^{d} represents one or more substituents selected from —OH, —NH₂, -halo, —CF₃, —CN, —COOR^{a}, —SR^{b};
R^{e} is acyl; and
R^{f} is a direct bond or an alkylene group,
or a salt, hydrate, pro-drug, isomer, tautomer and/or derivative thereof.

36. A compound of Formula (X) 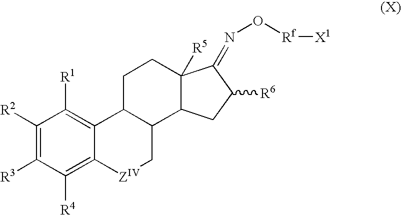
in which
R¹ and R⁴ are each selected from the group consisting of H, R^{a}, —R^{c}R^{d}, —CN, —NO₂, -halo, OH, —OR^{a}, —OC(O)R^{a};
R² is selected from the group consisting of —OR^{b}, —(R^{c})ₙ-AR^{b}, —H, —R^{e}, —R^{c}R^{e}, —CH═NOH, —CH═NOR^{b}, —CH═NNR^{b}₂, —OH, —SR^{b}, —R^{b}, —CN, —R^{c}R^{d} and -halo, in which n is 0 or 1;
R³ is selected from the group consisting of —OH, —OR^{a}, —R^{c}OR^{b}, —H, -ester-R^{b};
R⁵ is methyl;
R⁶ is —H, —OH, —OR^{b} or -halo;
X¹ is selected from aryl groups substituted by one or more substituents Y¹, wherein Y¹ is selected from —NO₂, —CN, —SO₃H, —SO₃R^{a}, —CO—R^{b}, —⁺NR^{b}₃, —CO₂R^{b}, -halo, —CF₃, —CCl₃, tetrazole, imidazole, -aryl, -substituted aryl, —R^{a}, —NH₂, —NR^{a}₂, —OH, —OR^{a}, —R^{c}—CN, —R^{c}-halo, —NR^{b}COR^{b}, —R^{c}—NR^{b}₂, —R^{c}R^{d}; and heteroaromatic groups substituted by one or more substituents Y, wherein Y is selected from —H, —NO₂, —CN, —SO₃H, SO₃R^{a}, —CO—R^{b}, —⁺NR^{b}₃, —CO₂R^{b}, -halo, —CF₃, —CCl₃, tetrazole, imidazole, -aryl, -substituted aryl, —R^{a}, —NH₂, NR^{a}₂, —OH, —OR^{a}, —R^{c}—CN, —R^{c}-halo, —NR^{b}COR^{b}, —R^{c}—NR^{b}₂, —R^{c}R^{d};
Z^{IV} is >CH₂, >C═O or >C═N—OH;
R^{a} is a straight chained, branched or cyclic alkyl, alkenyl, alkynyl, aralkyl, aralkenyl or aralkynyl;
R^{b} is H or straight chained, branched or cyclic alkyl, alkenyl, alkynyl, aralkyl, aralkenyl or aralkynyl;
R^{c} is a straight chained or branched C1-C10 alkylene, alkenylene or alkynylene;
R^{d} represents one or more substituents selected from —OH, —NH₂, -halo, —CF₃, —CN, —COOR^{a}, —SR^{b};
R^{e} is acyl; and
R^{f} is a direct bond or an alkylene group,
or a salt, hydrate, pro-drug, isomer, tautomer and/or derivative thereof.

37. A compound of Formula (XI) 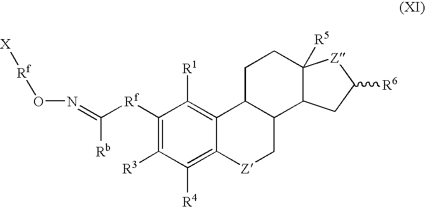
in which:
R¹ and R⁴ are each selected from the group consisting of H, R^{a}, —R^{c}R^{d}, —CN, —NO₂, -halo, OH, —OR^{a}, —OC(O)R^{a};
R² is selected from the group consisting of —OR^{b}, —(R^{c})ₙ-AR^{b}, —H, —R^{e}, —R^{c}R^{e}, —CH═NOH, —CH═NOR^{b}, —CH═NNR^{b}₂, —OH, —SR^{b}, —R^{b}, —CN, —R^{c}R^{d} and -halo, in which n is 0 or 1;
R³ is selected from the group consisting of —OH, —OR^{a}, —R^{c}OR^{b}, —H, -ester-R^{b};
R⁵ is methyl;
R⁶ is —H, —OH, —OR^{b} or -halo;
Z′ is A or >CH₂, >C═O, >C═N—OH, >C═N—OR^{b}, >C(R^{b})—OH, >C(R^{b})—CN; >C(R^{b})—NR^{b}₂, >CR^{b}₂, >C═N—NH₂, >C═N—NR^{b}₂, —O—, >N—R^{b}, >C(R^{b})—R^{c}—OR^{b}, >CR^{b}R^{e}, >CR^{b}—NR^{b}R^{e}, >C═N-ester-R^{a};
Z″ is A or >C═O, >C(H)OH, >C═N—OH, >C═N—OR^{b}, >C(R^{b})—OR^{b}, >C(R^{b})—R^{c}—OR^{b}, >C(H)—NR^{b}₂, >C(H)-halo, >CR^{b}₂, >C═N-ester-R^{a};
A is >C═N—O—X, >C═N—O—R^{c}—X, >C═N—NH—R^{c}—X, >C═N—NH—X, >C═N-ester-X;
X is an aromatic group substituted by one or more substituents Y, wherein Y is selected from —H, —NO₂, —CN, —SO₃H, —SO₃R^{a}, CO—R^{b}, —⁺NR^{b}₃, —CO₂R^{b}, -halo, —CF₃, —CCl₃, tetrazole, imidazole, -aryl, -substituted aryl, —R^{a}, —NH₂, —NR^{a}₂, —OH, —OR^{a}, —R^{c}—CN, —R^{c}-halo, —NR^{b}COR^{b}, —R^{c}—NR^{b}₂, —R^{c}R^{d};
R^{a} is a straight chained, branched or cyclic alkyl, alkenyl, alkynyl, aralkyl, aralkenyl or aralkynyl;
R^{b} is H or straight chained, branched or cyclic alkyl, alkenyl, alkynyl, aralkyl, aralkenyl or aralkynyl;
R^{c} is a straight chained or branched C1-C10 alkylene, alkenylene or alkynylene;
R^{d} represents one or more substituents selected from —OH, —NH₂, -halo, —CF₃, —CN, —COOR^{a}, —SR^{b};
R^{e} is acyl; and
R^{f} is a direct bond or an alkylene group
or a salt, hydrate, pro-drug, isomer, tautomer and/or derivative thereof.

38. A compound according to claim 1 selected from the group consisting of:
2-methoxy-6-(4-nitrobenzyloxy)iminoestradiol
2-methoxy-6-(4-nitrobenzyloxy)iminoestrone-17-oxime
2-methoxy-6-(3-nitrobenzyloxy)iminoestradiol
2-methoxy-6-(3-nitrobenzyloxy)iminoestrone-17-oxime
2-methoxy-6-(2-nitrobenzyloxy)iminoestradiol
2-methoxy-6-(2-nitrobenzyloxy)iminoestrone-17-oxime
2-methoxy-6-(2,4-dinitrophenylhydrazono)estrone-17-oxime
2-methoxy-6-(3-trifluoromethylbenzyloxy)iminoestrone-17-oxime
2-methoxy-6-(4-pyridylmethyloxy)iminoestrone-17-oxime
2-methoxy-6-(4-pyridylmethyloxy)iminoestradiol
6-(3,5-difluorobenzyloxy)imino-2-methoxyestrone-17-oxime
6-(3,5-difluorobenzyloxy)imino-2-methoxyestradiol
6-(4-cyanobenzyloxy)imino-2-methoxyestradiol
2-methoxy-6-(3-cyanobenzyloxy)iminoestrone-17-oxime
2-methoxy-6-(3-cyanobenzyloxy)iminoestradiol
6-(4-cyanobenzyloxy)imino-2-methoxyestrone-17-oxime
estrone-17-(4-nitrobenzyl)oxime
estrone-17-(3-nitrobenzyl)oxime
2-methoxyestrone-17-(4-nitrobenzyl)oxime
2-methoxyestrone-17-(3-nitrobenzyl)oxime
2-methoxy-6-(4-methoxybenzyloxy)-iminoestradiol
2-methoxy-6-(4-methoxybenzyloxy)-iminoestrone-17-oxime
estrone-17-(4-methoxybenzyl)oxime
2-methoxyestrone-17-(4-methoxybenzyl)oxime
2-methoxy-6-(4-trifluoromethylthiobenzyloxy)iminoestradiol
2-methoxy-6-(3-methoxybenzyloxy)iminoestrone-17-oxime
2-methoxy-6-(4-trifluoromethoxybenzyloxy)iminoestrone-17-oxime
2-methoxy-6-(3-methoxybenzyloxy)iminoestradiol
2-methoxy-6-(3-trifluoromethoxybenzyloxy)iminoestradiol
2-methoxy-6-(4-trifluoromethoxybenzyloxy)iminoestradiol
2-methoxy-6-(4-trifluoromethoxybenzyloxy)iminoestrone-17-oxime
2-methoxy-6-(4-trifluoromethylthiobenzyloxy)iminoestrone-17-oxime
6-(3,5-difluorobenzyloxy)imino-2-methoxyestrone-17-methyloxime
6-(4-nitrobenzyloxy)imino-2-methoxyestrone-17-methyloxime
2-methoxy-6-(4-methylbenzyloxy)iminoestrone-17-methyloxime
2-methoxy-6-(4-isopropylbenzyloxy)iminoestrone-17-oxime
2-methoxy-6-(4-methylbenzyloxy)iminoestradiol
6-(3,5-difluorobenzyloxy)iminoestriol
2-Methoxy-6-(4-nitrobenzyloxy)iminoestradiol-17-acetate
6-(3,5-difluorobenzyloxy)imino-17-ethinyl-estradiol.

39. A method of synthesising a compound of Formula I, as defined above, comprising the step of reacting a ketone or aldehyde precursor of Formula II, III or IV: 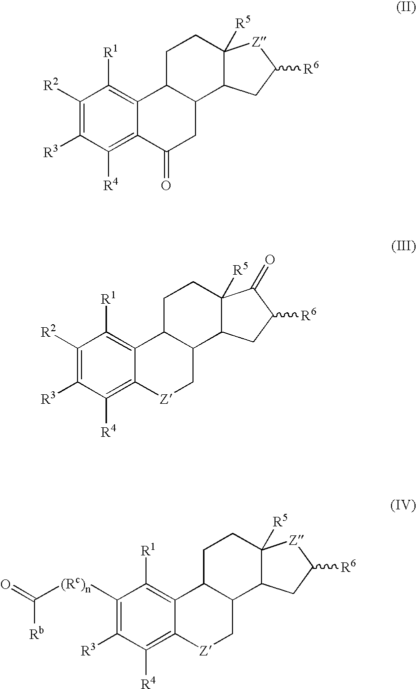
in which
R¹ and R⁴ are each selected from the group consisting of H, R^{a}, —R^{c}R^{d}, —CN, —NO₂, -halo, OH, —OR^{a}, —OC(O)R^{a};
R² is selected from the group consisting of —OR^{b}, —(R^{c})ₙ-AR^{b}, —H, —R^{e}, —R^{c}R^{e}, —CH═NOH, —CH═NOR^{b}, —CH═NNR^{b}₂, —OH, —SR^{b}, —R^{b}, —CN, —R^{c}R^{d} and -halo, in which n is 0 or 1;
R³ is selected from the group consisting of —OH, —OR^{a}, —R^{c}OR^{b}, —H, -ester-R^{b};
R⁵ is methyl;
R⁶ is —H, —OH, —OR^{b} or -halo;
Z′ is A or >CH₂, >C═O, >C═N—OH, >C═N—OR^{b}, >C(R^{b})—OH, >C(R^{b})—CN; >C(R^{b})—NR^{b}₂, >CR^{b}₂, >C═N—NH₂, >C═N—NR^{b}₂, —O—, >N—R^{b}, >C(R^{b})—R^{c}—OR^{b}, >CR^{b}R^{e}, >CR^{b}—NR^{b}R^{e}, >C═N-ester-R^{a};
Z″ is A or >C═O, >C(H)OH, >C═N—OH, >C═N—OR^{b}, >C(R^{b})—OR^{b}, >C(R^{b})—R^{c}—OR^{b}, >C(H)—NR^{b}₂, >C(H)-halo, >CR^{b}₂, >C═N-ester-R^{a};
A is >C═N—O—X, >C═N—O—R^{c}—X, >C═N—NH—R^{c}—X, >C═N—NH—X, >C═N-ester-X;
R^{a} is a straight chained, branched or cyclic alkyl, alkenyl, alkynyl, aralkyl, aralkenyl or aralkynyl;
R^{b} is H or straight chained, branched or cyclic alkyl, alkenyl, alkynyl, aralkyl, aralkenyl or aralkynyl;
R^{c} is a straight chained or branched C1-C10 alkylene, alkenylene or alkynylene;
R^{d} represents one or more substituents selected from —OH, —NH₂, -halo, —CF₃, —CN, —COOR^{a}, —SR^{b};
R^{e} is acyl;
or a salt, hydrate, pro-drug, isomer, tautomer and/or derivative thereof,
with an amine of the formula H₂N—O—X, H₂N—O—R^{c}—X, H₂N—NH—R^{c}—X, H₂N—NH—X or H₂N-ester-X,
in which X is an aromatic group substituted by one or more substituents Y, wherein Y is selected from —H, —NO₂, —CN, —SO₃H₁—SO₃R^{a}, —CO—R^{b}, —⁺NR³, —CO₂R^{b}, -halo, —CF₃, —CCl₃, tetrazole, imidazole, -aryl, -substituted aryl, —R^{a}, —NH₂, —NR^{a}₂, —OH, —OR^{a}, —R^{c}—CN, —R^{c}-halo, —NR^{b}COR^{b}, —R^{c}—NR^{b}₂, —R^{c}R^{d};
to form the compound of Formula I.

40. A pharmaceutical composition comprising compound of the Formula I: 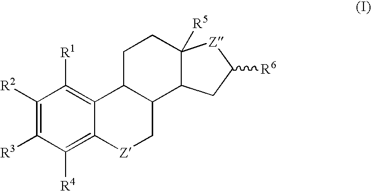
in which:
R¹ and R⁴ are each selected from the group consisting of H, R^{a}, —R^{c}R^{d}, —CN, —NO₂, -halo, OH, —OR^{a}, —OC(O) R^{a};
R² is selected from the group consisting of —OR^{b}, —(R^{c})ₙ-AR^{b}, —H, —R^{e}, —R^{c}R^{e}, —CH═NOH, —CH═NOR^{b}, —CH═NNR^{b}₂, —OH, —SR^{b}, —R^{b}, —CN, —R^{c}R^{d} and -halo, in which n is 0 or 1;
R³ is selected from the group consisting of —OH, —OR^{a}, —R^{c}OR^{b}, —H, -ester-R^{b};
R⁵ is methyl;
R⁶ is —H, —OH, —OR^{b} or -halo;
Z′ is A or >CH₂, >C═O, >C═N—OH, >C═N—OR^{b}, >C(R^{b})—OH, >C(R^{b})—CN; >C(R^{b})—NR^{b}₂, >CR^{b}₂, >C═N—NH₂, >C═N—NR^{b}₂, —O—, >N—R^{b}, >C(R^{b})—R^{c}—OR^{b}, >CR^{b}R^{e}, >CR^{b}—NR^{b}R^{e}, >C═N-ester-R^{a};
Z″ is A or >C═O, >C(H)OH, >C═N—OH, >C═N—OR^{b}, >C(R^{b})—OR^{b}, >C(R^{b})—R^{c}—OR^{b}, >C(H)—NR^{b}₂, >C(H)-halo, >CR^{b}₂, >C═N-ester-R^{a};
A is >C═N—O—X, >C═N—O—R^{c}—X, >C═N—NH—R^{c}—X, >C═N—NH—X, >C═N-ester-X;
X is an aromatic group substituted by one or more substituents Y, wherein Y is selected from —H, —NO₂, —CN, —SO₃H, SO₃R^{a}, —CO—R^{b}, —⁺NR^{b}₃, —CO₂R^{b}, -halo, —CF₃, —CCl₃, tetrazole, imidazole, -aryl, -substituted aryl, —R^{a}, —NH₂, —NR^{a}₂, —OH, —OR^{a}, —R^{c}—CN, —R^{c}-halo, —NR^{b}COR^{b}, —R^{c}—NR^{b}₂, —R^{c}R^{d};
R^{a} is a straight chained, branched or cyclic alkyl, alkenyl, alkynyl, aralkyl, aralkenyl or aralkynyl;
R^{b} is H or straight chained, branched or cyclic alkyl, alkenyl, alkynyl, aralkyl, aralkenyl or aralkynyl;
R^{c} is a straight chained or branched C1-C10 alkylene, alkenylene or alkynylene;
R^{d} represents one or more substituents selected from —OH, —NH₂, -halo, —CF₃, —CN, —COOR^{a}, —SR^{b};
R^{e} is acyl;
with the proviso the compound contains at least one group A,
or a salt, hydrate, pro-drug, isomer, tautomer and/or derivative thereof,
and a pharmaceutically acceptable carrier.

41. A method of treating a condition associated with smooth muscle cell and/or fibroblast function, which comprises administering a therapeutically effective amount of compound of the Formula I: 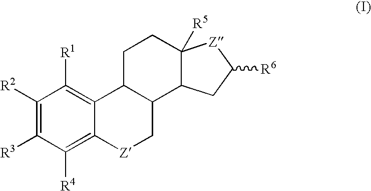
in which:
R¹ and R⁴ are each selected from the group consisting of H, R^{a}, —R^{c}R^{d}, —CN, —NO₂, -halo, OH, —OR^{a}, —OC(O)R^{a};
R² is selected from the group consisting of —OR^{b}, —(R^{c})ₙ-AR^{b}, —H, —R^{e}, —R^{c}R^{e}, —CH═NOH, —CH—NOR^{b}, —CH═NNR^{b}₂, —OH, —SR^{b}, —R^{b}, —CN, —R^{c}R^{d} and -halo, in which n is 0 or 1;
R³ is selected from the group consisting of —OH, —OR^{a}, —R^{c}OR^{b}, —H, -ester-R^{b};
R⁵ is methyl;
R⁶ is —H, —OH, —OR^{b} or -halo;
Z′ is A or >CH₂, >C═O, >C═N—OH, >C═N—OR^{b}, >C(R^{b})—OH, >C(R^{b})—CN; >C(R^{b})—NR^{b}₂, >CR^{b}₂, >C═N—NH₂, >C═N—NR^{b}₂, —O—, >N—R^{b}, >C(R^{b})—R^{c}—OR^{b}, >CR^{b}R^{e}, >CR^{b}—NR^{b}R^{e}, >C═N-ester-R^{a};
Z″ is A or >C═O, >C(H)OH, >C═N—OH, >C═N—OR^{b}, >C(R^{b})—OR^{b}, >C(R^{b})—R^{c}—OR^{b}, >C(H)—NR^{b}₂, >C(H)-halo, >CR^{b}₂, >C═N-ester-R^{a};
A is >C═N—O—X, >C═N—O—R^{c}—X, >C═N—NH—R^{c}—X, >C═N—NH—X, >C═N-ester-X;
X is an aromatic group substituted by one or more substituents Y, wherein Y is selected from —H, —NO₂, —CN, —SO₃H, —SO₃R^{a}, —CO—R^{b}, —⁺NR^{b}₃, —CO₂R^{b}, -halo, —CF₃, —CCl₃, tetrazole, imidazole, -aryl, -substituted aryl, —R^{a}, —NH₂, —NR^{a}₂, —OH, —OR^{a}, —R^{c}—CN, —R^{c}-halo, —NR^{b}COR^{b}, —R^{c}—NR^{b}₂, —R^{c}R^{d};
R^{a} is a straight chained, branched or cyclic alkyl, alkenyl, alkynyl, aralkyl, aralkenyl or aralkynyl;
R^{b} is H or straight chained, branched or cyclic alkyl, alkenyl, alkynyl, aralkyl, aralkenyl or aralkynyl;
R^{c} is a straight chained or branched C1-C10 alkylene, alkenylene or alkynylene;
R^{d} represents one or more substituents selected from —OH, —NH₂, -halo, —CF₃, —CN, —COOR^{a}, —SR^{b};
R^{e} is acyl;
with the proviso the compound contains at least one group A,
or a salt, hydrate, pro-drug, isomer, tautomer and/or derivative thereof,
to a subject in need thereof.

42. A method according to claim 41, in which the smooth muscle cell and/or fibroblast function is cell proliferation.

43. A method according to claim 41, in which the smooth muscle cell and/or fibroblast function is cell cytokine expression.

44. A method according to claim 43, in which the cell cytokine expression is GM-CSF expression.

45. A method according to claim 41, in which the smooth muscle cell and/or fibroblast function is cell extracellular matrix deposition.

46. A method according to claim 41, in which the smooth muscle cell and/or fibroblast function is cell contractility.

47. A method according to claim 41, in which the smooth muscle cell and/or fibroblast function is cell migration.

48. A method according to claim 41, in which the smooth muscle cells are airway smooth muscle cells.

49. A method according to claim 41, in which the fibroblasts are airway fibroblasts.

50. A method according to claim 41, in which the condition is airway hyperresponsiveness.

51. A method according to claim 50, in which the airway hyperresponsiveness is associated with asthma.

52. A method according to claim 41, in which the condition is fibrosis.

53. A method according to claim 41, in which the condition is pulmonary fibrosis.

54. A method of treating inflammation, which comprises administering a therapeutically effective amount of compound of the formula I: 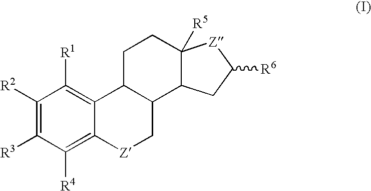
in which:
R¹ and R⁴ are each selected from the group consisting of H, R^{a}, —R^{c}R^{d}, —CN, —NO₂, -halo, OH, —OR^{a}, —OC(O)R^{a};
R² is selected from the group consisting of —OR^{b}, —(R^{c})ₙ-AR^{b}, —H, —R^{e}, —R^{c}R^{e}, —CH═NOH, —CH═NOR^{b}, —CH═NNR^{b}₂, —OH, —SR^{b}, —R^{b}, —CN, —R^{c}R^{d} and -halo, in which n is 0 or 1;
R³ is selected from the group consisting of —OH, —OR^{a}, —R^{c}OR^{b}, —H, -ester-R^{b};
R⁵ is methyl;
R⁶ is —H, —OH, —OR^{b} or -halo;
Z′ is A or >CH₂, >C═O, >C═N—OH, >C═N—OR^{b}, >C(R^{b})—OH, >C(R^{b})—CN; >C(R^{b})—NR^{b}₂, >CR^{b}₂, >C═N—NH₂, >C═N—NR^{b}₂, —O—, >N—R^{b}, >C(R^{b})—R^{c}—OR^{d}, >CR^{b}R^{e}, >CR^{b}—NR^{b}R^{e}, >C═N-ester-R^{a};
Z″ is A or >C═O, >C(H)OH, >C═N—OH, >C═N—OR^{b}, >C(R^{b})—OR^{b}, >C(R^{b})—R^{c}—OR^{b}, >C(H)—NR^{b}₂, >C(H)-halo, >CR^{b}₂, >C═N-ester-R^{a};
A is >C═N—O—X, >C═N—O—R^{c}—X, >C═N—NH—R^{c}—X, >C═N—NH—X, >C═N-ester-X;
X is an aromatic group substituted by one or more substituents Y, wherein Y is selected from —H, —NO₂, —CN, —SO₃H, —SO₃R^{a}, —CO—R^{b}, —⁺NR^{b}₃, —CO₂R^{b}, -halo, —CF₃, —CCl₃, tetrazole, imidazole, -aryl, -substituted aryl, —R^{a}, —NH₂, —NR^{a}₂, —OH, —OR^{a}, —R^{c}—CN, —R^{c}-halo, —NR^{b}COR^{b}, —R^{c}—NR^{b}₂, —R^{c}R^{d};
R^{a} is a straight chained, branched or cyclic alkyl, alkenyl, alkynyl, aralkyl, aralkenyl or aralkynyl;
R^{b} is H or straight chained, branched or cyclic alkyl, alkenyl, alkynyl, aralkyl, aralkenyl or aralkynyl;
R^{c} is a straight chained or branched C1-C10 alkylene, alkenylene or alkynylene;
R^{d} represents one or more substituents selected from —OH, —NH₂, -halo, —CF₃, —CN, —COOR^{a}, —SR^{b};
R^{e} is acyl;
with the proviso the compound contains at least one group A,
or a salt, hydrate, pro-drug, isomer, tautomer and/or derivative thereof,
to a subject in need thereof.

55. Use of a compound of Formula I: 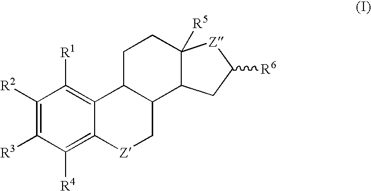
in which:
R¹ and R⁴ are each selected from the group consisting of H, R^{a}, —R^{c}R^{d}, —CN, —NO₂, -halo, OH, —OR^{a}, —OC(O)R^{a};
R² is selected from the group consisting of —OR^{b}, —(R^{c})ₙ-AR^{b}, —H, —R^{e}, —R^{c}R^{e}, —CH═NOH, —CH═NOR^{b}, —CH═NNR^{b}, —OH, —SR^{b}, —R^{b}, —CN, —R^{c}R^{d} and -halo, in which n is 0 or 1;
R³ is selected from the group consisting of —OH, —OR^{a}, —R^{c}OR^{b}, —H, -ester-R^{b};
R⁵ is methyl;
R⁶ is —H, —OH, —OR^{b} or -halo;
Z′ is A or >CH₂, >C═O, >C═N—OH, >C═N—OR^{b}, >C(R^{b})—OH, >C(R^{b})—CN; >C(R^{b})—NR^{b}₂, >CR^{b}₂, >C═N—NH₂, >C═N—NR^{b}₂, —O—, >N—R^{b}, >C(R^{b})—R^{c}—OR^{b}, >CR^{b}R^{e}, >CR^{b}—NR^{b}R^{e}, >C═N-ester-R^{a};
Z″ is A or >C═O, >C(H)OH, >C═N—OH, >C═N—OR^{b}, >C(R^{b})—OR^{b}, >C(R^{b})—R^{c}—OR^{b}, >C(H)—NR^{b}₂, >C(H)-halo, >CR^{b}₂, >C═N-ester-R^{a};
A is >C═N—O—X, >C═N—O—R^{c}—X, >C═N—NH—R^{c}—X, >C═N—NH—X, >C═N-ester-X;
X is an aromatic group substituted by one or more substituents Y, wherein Y is selected from —H, —NO₂, —CN, —SO₃H, —SO₃R^{a}, CO—R^{b}, —⁺NR^{b}₃, —CO₂R^{b}, -halo, —CF₃, —CCl₃, tetrazole, imidazole, -aryl, -substituted aryl, —R^{a}, —NH₂, —NR^{a}₂, —OH, —OR^{a}, —R^{c}—CN, —R^{c}-halo, —NR^{b}COR^{b}, —R^{c}—NR^{b}₂, —R^{c}R^{d};
R^{a} is a straight chained, branched or cyclic alkyl, alkenyl, alkynyl, aralkyl, aralkenyl or aralkynyl;
R^{b} is H or straight chained, branched or cyclic alkyl, alkenyl, alkynyl, aralkyl, aralkenyl or aralkynyl;
R^{c} is a straight chained or branched C1-C10 alkylene, alkenylene or alkynylene;
R^{d} represents one or more substituents selected from —OH, —NH₂, -halo, —CF₃, —CN, —COOR^{a}, —SR^{b};
R^{e} is acyl;
with the proviso the compound contains at least one group A,
or a salt, hydrate, pro-drug, isomer, tautomer and/or derivative thereof,
in the manufacture of a medicament for treating a condition associated with smooth muscle cell and/or fibroblast function.

56. Use of compound of formula I: 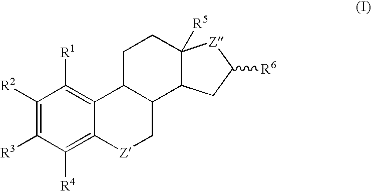
in which:
R¹ and R⁴ are each selected from the group consisting of H, R^{a}, —R^{c}R^{d}, —CN, —NO₂, -halo, OH, —OR^{a}, —OC(O)R^{a};
R² is selected from the group consisting of —OR^{b}, —(R^{c})ₙ-AR^{b}, —H, R^{e}, —R^{c}R^{e}, —CH═NOH, —CH═NOR^{b}, —CH═NNR^{b}₂, —OH, —SR^{b}, —R^{b}, —CN, —R^{c}R^{d} and -halo, in which n is 0 or 1;
R³ is selected from the group consisting of —OH, —OR^{a}, —R^{c}OR^{b}, —H, -ester-R^{b};
R⁵ is methyl;
R⁶ is —H, —OH, —OR^{b} or -halo;
Z′ is A or >CH₂, >C═O, >C═N—OH, >C═N—OR^{b}, >C(R^{b})—OH, >C(R^{b})—CN; >C(R^{b})—NR^{b}₂, >CR^{b}₂, >C═N—NH₂, >C═N—NR^{b}₂, —O—, >N—R^{b}, >C(R^{b})—R^{c}OR^{b}, >CR^{b}R^{e}, >CR^{b}—NR^{b}R^{e}, >C═N-ester-R^{a};
Z″ is A or >C═O, >C(H)OH, >C═N—OH, >C═N—OR^{b}, >c(R^{b})—OR^{b}, >C(R^{b})—R^{c}—OR^{b}, >C(H)—NR^{b}₂, >C(H)-halo, >CR^{b}₂, >C═N-ester-R^{a};
A is >C═N—O—X, >C═N—O—R^{c}—X, >C═N—NH—R^{c}—X, >C═N—NH—X, >C═N-ester-X;
X is an aromatic group substituted by one or more substituents Y, wherein Y is selected from —H, —NO₂, —CN, —SO₃H, —SO₃R^{a}, —CO—R^{b}, —⁺NR^{b}₃, —CO₂R^{b}, -halo, —CF₃, —CCl₃, tetrazole, imidazole, -aryl, -substituted aryl, —R^{a}, —NH₂, —NR^{a}₂, —OH, —OR^{a}, —R^{c}—CN, —R^{c}-halo, —NR^{b}COR^{b}, —R^{c}—NR^{b}₂, —R^{c}R^{d};
R^{a} is a straight chained, branched or cyclic alkyl, alkenyl, alkynyl, aralkyl, aralkenyl or aralkynyl;
R^{b} is H or straight chained, branched or cyclic alkyl, alkenyl, alkynyl, aralkyl, aralkenyl or aralkynyl;
R^{c} is a straight chained or branched C1-C10 alkylene, alkenylene or alkynylene;
R^{d} represents one or more substituents selected from —OH, —NH₂, -halo, —CF₃, —CN, —COOR^{a}, —SR^{b};
R^{e} is acyl;
with the proviso the compound contains at least one group A,
or a salt, hydrate, pro-drug, isomer, tautomer and/or derivative thereof,
in the manufacture of a medicament for treating an inflammatory condition.

57. Use of a compound of Formula I: 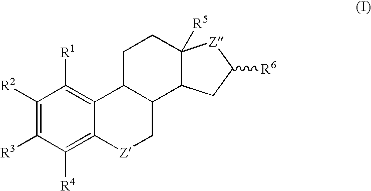
in which:
R¹ and R⁴ are each selected from the group consisting of H, R^{a}, —R^{c}R^{d}, —CN, —NO₂, -halo, OH, —OR^{a}, —OC(O)R^{a};
R² is selected from the group consisting of —OR^{b}, —(R^{c})ₙ-AR^{b}, —H, —R^{e}, —R^{c}R^{e}, —CH═NOH, —CH═NOR^{b}, —CH═NNR^{b}₂, —OH, —SR^{b}, —R^{b}, —CN, —R^{c}R^{d} and -halo, in which n is 0 or 1;
R³ is selected from the group consisting of —OH, —OR^{a}, —R^{c}OR^{b}, —H, -ester-R^{b};
R⁵ is methyl;
R⁶ is —H, —OH, —OR^{b} or -halo;
Z′ is A or >CH₂, >C═O, >C═N—OH, >C═N—OR^{b}, >C(R^{b})—OH, >C(R^{b})—CN; >C(R^{b})—NR^{b}₂, >CR^{b}₂, >C═N—NH₂, >C═N—NR^{b}₂, —O—, >N—R^{b}, >C(R^{b})—R^{c}—OR^{b}, >CR^{b}R^{e} >CR^{b}—NR^{b}R^{e}, >C═N-ester-R^{a};
Z″ is A or >C═O, >C(H)OH, >C═N—OH, >C═N—OR^{b}, >c(R^{b})—OR^{b}, >C(R^{b})—R^{c}—OR^{b}, >C(H)—NR^{b}₂, >C(H)-halo, >CR^{b}₂, >C═N-ester-R^{a};
A is >C═N—O—X, >C═N—O—R^{c}—X, >C═N—NH—R^{c}—X, >C═N—NH—X, >C═N-ester-X;
X is an aromatic group substituted by one or more substituents Y, wherein Y is selected from —H, —NO₂, —CN, —SO₃H, —SO₃R^{a}, —CO—R^{b}, —⁺NR^{b}₃, —CO₂R^{b}, -halo, —CF₃, —CCl₃, tetrazole, imidazole, -aryl, -substituted aryl, —R^{a}, —NH₂, —NR^{a}₂, —OH, —OR^{a}, —R^{c}—CN, —R^{c}-halo, —NR^{b}COR^{k}, —R^{c}—NR^{b}₂, —R^{c}R^{d};
R^{e} is a straight chained, branched or cyclic alkyl, alkenyl, alkynyl, aralkyl, aralkenyl or aralkynyl;
R^{b} is H or straight chained, branched or cyclic alkyl, alkenyl, alkynyl, aralkyl, aralkenyl or aralkynyl;
R^{c} is a straight chained or branched C1-C10 alkylene, alkenylene or alkynylene;
R^{d} represents one or more substituents selected from —OH, —NH₂, -halo, —CF₃, —CN, —COOR^{a}, —SR^{b};
R^{e} is acyl;
with the proviso the compound contains at least one group A,
or a salt, hydrate, pro-drug, isomer, tautomer and/or derivative thereof,
as an agent for modulating smooth muscle cell and/or fibroblast function.

58. Use of a compound of Formula I: 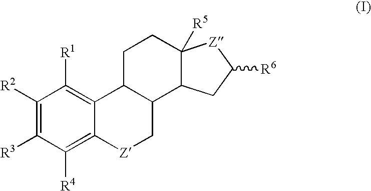
in which:
R¹ and R⁴ are each selected from the group consisting of H, R^{a}, —R^{c}R^{d}, —CN, —NO₂, -halo, OH, —OR^{a}, —OC(O)R^{a};
R² is selected from the group consisting of —OR^{b}, —(R^{c})ₙ-AR^{b}, —H, —R^{e}, —R^{c}R^{e}, —CH═NOH, —CH═NOR^{b}, —CH═NNR^{b}₂, —OH, —SR^{b}, —R^{b}, —CN, —R^{c}R^{d} and -halo, in which n is 0 or 1;
R³ is selected from the group consisting of —OH, —OR^{a}, —R^{c}OR^{b}, —H, -ester-R^{b};
R⁵ is methyl;
R⁶ is —H, —OH, —OR^{b} or -halo;
Z′ is A or >CH₂, >C═O, >C═N—OH, >C═N—OR^{b}, >C(R^{b})—OH, >C(R^{b})—CN; >C(R^{b})—NR^{b}₂, >CR^{b}₂, >C═N—NH₂, >C═N—NR^{b}₂, —O—, >N—R^{b}, >C(R^{b})—R^{c}—OR^{b}, >CR^{b}R^{e}, >CR^{b}—NR^{b}R^{e}, >C═N-ester-R^{a};
Z″ is A or >C═O, >C(H)OH, >C═N—OH, >C═N—OR^{b}, >C(R^{b})—OR^{b}, >C(R^{b})—R^{c}—OR^{b}, >C(H)NR^{b}₂, >C(H)-halo, >CR^{b}₂, >C═N-ester-R^{a}.;
A is >C═N—O—X, >C═N—O—R^{c}—X, >C═N—NH—R^{c}—X, >C═N—NH—X, >C═N-ester-X;
X is an aromatic group substituted by one or more substituents Y, wherein Y is selected from —H, —NO₂, —CN, —SO₃H, —SO₃R^{a}, —CO—R^{b}, —⁺NR^{b}₃, —CO₂R^{b}, -halo, —CF₃, —CCl₃, tetrazole, imidazole, -aryl, -substituted aryl, —R^{a}, —NH₂, —NR^{a}₂, —OH, —OR^{a}, —R^{c}—CN, —R^{c}-halo, —NR^{b}COR^{b}, —R^{c}—NR^{b}₂, —R^{c}R^{d};
R^{a} is a straight chained, branched or cyclic alkyl, alkenyl, alkynyl, aralkyl, aralkenyl or aralkynyl;
R^{b} is H or straight chained, branched or cyclic alkyl, alkenyl, alkynyl, aralkyl, aralkenyl or aralkynyl;
R^{c} is a straight chained or branched C1-C10 alkylene, alkenylene or alkynylene;
R^{d} represents one or more substituents selected from —OH, —NH₂, -halo, —CF₃, —CN, —COOR^{a}, —SR^{b};
R^{e} is acyl;
with the proviso the compound contains at least one group A,
or a salt, hydrate, pro-drug, isomer, tautomer and/or derivative thereof,
as an agent for treating an inflammatory condition.
